SEQUENCE LISTING (259 sequences)

SEQ ID NO: 1 - protein, 129 aa, synthetic construct
  REGION 1..129: note = Description of Artificial Sequence: Synthetic polypeptide
QVQLAETGGGLVQAGGSLRLSCSASGLTFGNYAMGWFRQAPGKEREFVASISRSGSNTWYAEPLKGRFAISRDNDKNALYLQMNSLKPEDTAVYYCAGGSYNSDWWNYMYWGQGTQVTVSSEPKTPKPQ

SEQ ID NO: 2 - DNA, 387 bp, synthetic construct
  misc_feature 1..387: note = Description of Artificial Sequence: Synthetic polynucleotide
  source 1..387: mol_type = other DNA
caggtgcagctggcggagacggggggaggattggtgcaggctgggggctcgctgagactctcctgttcagcctctgggctcaccttcgggaactatgccatgggctggttccgccaggctccagggaaggagcgtgagtttgtagcatctatttctcggagtggtagtaacacatggtatgcagaacccctgaagggccgattcgccatctccagagacaacgacaagaacgcgctctatctgcaaatgaacagcctgaaacctgaggacacggccgtttattactgtgctggaggatcttataatagtgactggtggaactatatgtactggggccaggggacccaggtcactgtctcctcagaacccaagacaccaaaaccacaa

SEQ ID NO: 3 - protein, 142 aa, synthetic construct
  REGION 1..142: note = Description of Artificial Sequence: Synthetic polypeptide
QVQLVESGGGGLVQAGGSLRLSCAASGRTFSGYAMGWFRQAPGKEREFVADISWSGHNTYYGDSVKGRFTISRDTAKNTVYLQMNSLKPEDTAVYYCAAEGARTHLSDSYYFPGLWAEPPVGYWGQGTQVTVSSEPKTPKPQ

SEQ ID NO: 4 - DNA, 426 bp, synthetic construct
  misc_feature 1..426: note = Description of Artificial Sequence: Synthetic polynucleotide
  source 1..426: mol_type = other DNA
caggtgcagctggtggagtcgggtgggggaggactggtgcaggctgggggctctctgagactctcctgtgcagcctctggacgcaccttcagtggctatgccatgggctggttccgccaggctccggggaaggagcgtgagtttgtagccgatattagctggagtggtcataacacgtactatggagactccgtgaagggccgattcaccatctccagagacaccgccaagaacacggtgtatctgcaaatgaacagcctgaaacctgaggacacggccgtttattactgtgcagcggagggggcccgtacacaccttagtgatagttactacttcccgggcctctgggccgaaccccccgtgggctactggggccaggggacccaggtcactgtctcctcagaacccaagacaccaaaaccacaa

SEQ ID NO: 5 - protein, 131 aa, synthetic construct
  REGION 1..131: note = Description of Artificial Sequence: Synthetic polypeptide
QVQLVETGGGLVQAGGTLRLSCAASGRTFTSYYIGWFRQEPGKEREFVASIGWTDDNTYYADSVKGRFTISRDNAETTAYLQMSGLKPEDTAVYYCAADYGSGIRAWYNWIYWGQGTQVTVSSEPKTPKPQ

SEQ ID NO: 6 - DNA, 393 bp, synthetic construct
  misc_feature 1..393: note = Description of Artificial Sequence: Synthetic polynucleotide
  source 1..393: mol_type = other DNA
caggtgcagctggtggagaccgggggaggattggtgcaggctgggggcactctgagactctcctgtgcagcctctggacgtaccttcacgagctattacattggctggttccgccaggaaccagggaaggagcgtgagtttgtagcaagtatcggctggaccgatgataacacatactatgcagactccgtgaagggccgattcaccatctccagagacaacgccgagaccacggcatatctgcaaatgtcgggcctgaaacctgaggacacggccgtttattactgtgcagccgactacgggtcagggatacgggcctggtataattggatttactggggccaggggacccaggtcaccgtctcctcagaacccaagacaccaaaaccacaa

SEQ ID NO: 7 - protein, 132 aa, synthetic construct
  REGION 1..132: note = Description of Artificial Sequence: Synthetic polypeptide
QLQLAETGGGLVQPGGSLRLSCAASGATLDTYIITWFRQAPGKEREAVSCINRSGSTTYSDSVKGRFTISRDNAQKTVYLQMNSLNPEDTAIYYCAADASYRTCGGSWWNWAYWGQGTQVTVSSEPKTPKPQ

SEQ ID NO: 8 - DNA, 396 bp, synthetic construct
  misc_feature 1..396: note = Description of Artificial Sequence: Synthetic polynucleotide
  source 1..396: mol_type = other DNA
cagttgcagctcgcggagacgggaggaggcttggtgcagcctggggggtctctgagactctcctgtgcagcctctggcgccactttggatacttatatcataacctggttccgccaggccccagggaaggagcgtgaggccgtctcatgtattaatcgtagtggtagcacgacctattcagactccgtgaagggccgattcaccatctccagagacaacgcccagaaaacggtgtatctgcagatgaacagcctgaaccctgaggacacagccatttattactgcgcagcggatgcttcgtaccgtacttgcggcgggagttggtggaattgggcgtactggggccaggggacccaggtcaccgtctcctcagaacccaagacaccaaaaccacaa

SEQ ID NO: 9 - protein, 130 aa, synthetic construct
  REGION 1..130: note = Description of Artificial Sequence: Synthetic polypeptide
QVQLAESGGGSVQPGGSLRLSCAASGFTFSSYTMSWVRQAPGKGIEWVSDINGGGDRTDYADSVKGRFTISRDNARNTLYLQMNSLQPEDTAVYYCAKDLSYVSGTYFANDWGQGTQVTVSSEPKTPKPQ

SEQ ID NO: 10 - DNA, 390 bp, synthetic construct
  misc_feature 1..390: note = Description of Artificial Sequence: Synthetic polynucleotide
  source 1..390: mol_type = other DNA
caggtgcagctcgcggagtctggaggaggctcggtgcaacctggggggtctctgagactctcctgtgcagcctctggattcaccttcagtagttatactatgagctgggtccgccaggctccaggaaaggggatcgagtgggtctcagatattaatgggggtggtgatagaacagactatgcagactccgtgaagggccgattcaccatctccagagacaacgccaggaacacgctgtatctgcaaatgaacagcctgcaacctgaggacacggccgtgtattactgtgcaaaagatctgagctacgttagtggtacttatttcgcgaacgactggggccaggggacccaggtcaccgtctcctccgaacccaagacaccaaaaccacaa

SEQ ID NO: 11 - protein, 126 aa, synthetic construct
  REGION 1..126: note = Description of Artificial Sequence: Synthetic polypeptide
QLQLAESGGGLVQPGGSLRLSCTASGIIFDYYSVDWYRQAPGKERELVATITGDGSPNYADSVKGRFTISRDNAKKTVYLQMNGLKPEETAVYYCHAKRTIGTKSEYWGQGTQVTVSSEPKTPKPQ

SEQ ID NO: 12 - DNA, 378 bp, synthetic construct
  misc_feature 1..378: note = Description of Artificial Sequence: Synthetic polynucleotide
  source 1..378: mol_type = other DNA
cagttgcagctggcggagtcggggggaggcttggtgcagcctggggggtctctgagactctcctgtacagcctctggaatcatcttcgattactattccgtggactggtaccgccaggctccagggaaggagcgcgaattggtcgcaactattacgggtgatggtagcccgaactatgcggactctgtcaagggccgattcaccatctccagagacaacgccaagaagacggtgtatctgcaaatgaacggcctgaaacctgaggaaacggccgtctattactgtcatgccaaaaggactatagggaccaaatctgagtactggggccaggggacccaggtcactgtctcctcagaacccaagacaccaaaaccacaa

SEQ ID NO: 13 - protein, 123 aa, synthetic construct
  REGION 1..123: note = Description of Artificial Sequence: Synthetic polypeptide
QVQLAETGGGLVQAGGSLRLSCLASRMSFSRRPMAWYRQAPGKQRERVATISSFGDTTNYTDSVEGRFTISRDNAKNTMYLQMNSLKPDDTAVYYCNTLLATYAWGQGTQVTVSSEPKTPKPQ

SEQ ID NO: 14 - DNA, 369 bp, synthetic construct
  misc_feature 1..369: note = Description of Artificial Sequence: Synthetic polynucleotide
  source 1..369: mol_type = other DNA
caggtgcagctcgcggagaccgggggaggcttggtgcaggctgggggttctctgagactctcctgtttagcctctagaatgagctttagtaggcgccccatggcctggtaccgccaggctccaggcaagcagcgcgaaagggtcgcaactattagtagtttcggtgataccacaaactatacagactccgtggagggccgattcaccatctccagggacaatgccaagaacacgatgtatctgcaaatgaacagcctgaaacctgacgacacggccgtgtattactgtaacacattactcgctacgtacgcctggggccaggggacccaggtcaccgtctcctcagaacccaagacaccaaaaccacaa

SEQ ID NO: 15 - protein, 135 aa, synthetic construct
  REGION 1..135: note = Description of Artificial Sequence: Synthetic polypeptide
QVQLAESGGGLVQAGGSLRLSCAASGRTFSSYVMGWFRQAPGKEREFVAAISRNGGKTYYADSVKGRFTISRDGTENTVYLQMNSLKPEDTAVYYCAAAVAASAEFVTARSNFYEYWGQGTQVTVSSEPKTPKPQ

SEQ ID NO: 16 - DNA, 405 bp, synthetic construct
  misc_feature 1..405: note = Description of Artificial Sequence: Synthetic polynucleotide
  source 1..405: mol_type = other DNA
caggtgcagctggcggagtcggggggaggattggtgcaggctgggggctctctgagactctcctgtgcagcctctggacgcaccttcagtagctatgtcatgggctggttccgccaggctccagggaaggagcgtgagtttgtggccgctattagccgaaatggtggtaagacctactatgcagactccgtgaagggccgattcaccatctcaagagacggcaccgagaacacggtgtatctgcaaatgaacagcctgaaacctgaggacacggccgtttattactgcgcagcagccgtagccgcttctgccgagtttgttacggctcgctcgaatttttatgaatattggggtcaggggacccaggtcactgtctcctcagaacccaagacaccaaaaccacaa

SEQ ID NO: 17 - protein, 132 aa, synthetic construct
  REGION 1..132: note = Description of Artificial Sequence: Synthetic polypeptide
QVQLVETGGGLVQAGGSLRLSCEASGSVVTIKEMGWYRQAPGKEREQERDLVAAIGIGGVTYYATSVKGRFTISRDSAKTTLRLQMSSLRPEDTAMYYCAVITDRNTGGYPDYWGQGTQVTVTAEPKTPKPQ

SEQ ID NO: 18 - DNA, 396 bp, synthetic construct
  misc_feature 1..396: note = Description of Artificial Sequence: Synthetic polynucleotide
  source 1..396: mol_type = other DNA
caggtgcagctggtggagacgggtggaggcttggtgcaggctggggggtctctgagactctcctgtgaagcctctggaagcgtcgtcaccatcaaagagatgggctggtaccgacaggctccaggaaaggagcgcgaacaggagcgcgacttggtcgcagcaattggcattggtggtgtcacatactacgcaacctctgtgaagggccgattcaccatctccagagacagtgccaagactacgctgcgtctgcaaatgagcagcctgagacctgaggacacggccatgtattattgtgcggtcataactgacaggaacaccggtggttacccggactactggggccaggggacccaggtcactgttaccgcagaacccaagacaccaaaaccacaa

SEQ ID NO: 19 - protein, 135 aa, synthetic construct
  REGION 1..135: note = Description of Artificial Sequence: Synthetic polypeptide
QVQLVESGGGLVQAGGSLRLSCAASILTYDLDYYYIGWVRQAPGKEREGVSCISSTDGATYYADSVKGRFTISRNNAKNTVYLQMNNLKPEDTAIYYCAAAPLAGRYCPASHEYGYWGQGTQVTVSSEPKTPKPQ

SEQ ID NO: 20 - DNA, 405 bp, synthetic construct
  misc_feature 1..405: note = Description of Artificial Sequence: Synthetic polynucleotide
  source 1..405: mol_type = other DNA
caggtgcagctggtggagtccggtggaggcttggtgcaggctggggggtctctgagactctcctgtgcagcctctatactcacttatgatttggattattattacataggctgggtccgccaggccccagggaaggagcgtgagggggtctcatgtattagtagtactgatggtgccacatactatgcagactccgtgaagggccgattcaccatctccagaaacaacgccaagaacacggtgtatctgcaaatgaacaacctaaaacctgaggacacagccatttattattgtgcagcagcccccctggctgggcgctactgtcccgcctcgcatgagtatggctactggggtcaggggacccaggtcaccgtctcgtcagaacccaagacaccaaaaccacaa

SEQ ID NO: 21 - protein, 133 aa, synthetic construct
  REGION 1..133: note = Description of Artificial Sequence: Synthetic polypeptide
QVQLVESGGGLVQPGESLRLSCGASGMSLDYYAIAWYRQAPGKEREGVSCISVSGSSAQYLDSVRGRFIISKDNTKSTAYLQMNSLKPEDTAVYYCAALADCAGYASLTFDFDSWGQGTQVAVSSAHHSEDPS

SEQ ID NO: 22 - DNA, 399 bp, synthetic construct
  misc_feature 1..399: note = Description of Artificial Sequence: Synthetic polynucleotide
  source 1..399: mol_type = other DNA
caggtgcagctcgtggagtcgggtggaggcttggtgcagcctggggagtctctgagactctcctgtggagcctctggaatgagtttggattactatgccatagcctggtaccgccaggccccagggaaggagcgtgagggggtctcatgtattagtgttagtggcagtagcgcacaatatttagactccgtgaggggtcgcttcatcatctccaaagacaacaccaagagcacggcgtatctgcaaatgaacagcctgaagcctgaagacacagccgtttattactgcgcagccctggccgactgtgcaggctatgccagtcttacctttgactttgattcttggggccaggggacccaggtcgccgtctcctcggcgcaccacagcgaagacccctcg

SEQ ID NO: 23 - protein, 133 aa, synthetic construct
  REGION 1..133: note = Description of Artificial Sequence: Synthetic polypeptide
QVQLVESGGGLVQAGGSLRLSCAPSRLTLDFFAIAWFRQAPGKEREGVSCISSHDGSTYYTDSVKGRFTISKDNAKNTVYLQMNSLKPEDTAVYYCALDHNVGTCQLTQAEYDYWGQGTQVTVSSAHHSEDPS

SEQ ID NO: 24 - DNA, 399 bp, synthetic construct
  misc_feature 1..399: note = Description of Artificial Sequence: Synthetic polynucleotide
  source 1..399: mol_type = other DNA
caggtgcagctggtggagtccggtggaggcttggtgcaggctggggggtctctgagactctcctgtgcaccctcgcgattaactttggatttctttgccatagcctggttccgccaggccccagggaaggagcgtgagggggtctcatgtattagtagtcatgatggtagcacatactacacagactccgtgaagggccgattcaccatctccaaagacaacgccaagaacacggtgtatctgcaaatgaacagcctgaagcctgaggacacagccgtttattactgtgccctagaccataacgtgggtacctgccaactcacccaagctgagtatgactactggggccaggggacccaggtcaccgtctcctcggcgcaccacagcgaagacccctcg

SEQ ID NO: 25 - protein, 125 aa, synthetic construct
  REGION 1..125: note = Description of Artificial Sequence: Synthetic polypeptide
QVQLVESGGGLVQSGGSLRLSCAASGSIDSLYHMGWYRQAPGKERELVARVQDGGSTAYKDSVKGRFTISRDFSRSTMYLQMNSLKPEDTAIYYCAAKSTISTPLSWGQGTQVTVSSEPKTPKPQ

SEQ ID NO: 26 - DNA, 375 bp, synthetic construct
  misc_feature 1..375: note = Description of Artificial Sequence: Synthetic polynucleotide
  source 1..375: mol_type = other DNA
caggtgcagctggtggagtccgggggaggcttggtgcagtctggggggtctctgagactctcctgtgcagcctctggaagtatcgatagtctctatcatatgggctggtaccgccaggctccagggaaggagcgcgagttggtcgcacgagttcaagatgggggtagcacagcgtacaaagactctgtgaaggggcgattcaccatctccagagacttttccaggagcacgatgtatctgcaaatgaacagcctgaaacctgaggacacggccatctattactgtgcggcgaagagtacaattagcacccccttgtcctggggccaggggacccaggtcaccgtctcctcggaacccaagacaccaaaaccacaa

SEQ ID NO: 27 - protein, 137 aa, synthetic construct
  REGION 1..137: note = Description of Artificial Sequence: Synthetic polypeptide
QVQLVESGGGLVQAGGSLRLSCAASGFTLGHNQVAWFRQAPGKEREGVACISATGASTHYADPVKGRFTVSRDNTKNVVYLQVNSLKPEDTANYVCASRFSLMSIDASMCLSAPQYDRWGQGTQVRISSEPKTPKPQ

SEQ ID NO: 28 - DNA, 411 bp, synthetic construct
  misc_feature 1..411: note = Description of Artificial Sequence: Synthetic polynucleotide
  source 1..411: mol_type = other DNA
caggtgcagctggtggagtccggtggaggcttggtgcaggctggggggtctctgagactctcctgtgcagcctctggattcactttgggacataatcaagtagcctggttccgccaggccccaggcaaggagcgtgagggggtcgcgtgtattagcgccaccggtgctagcacacactatgcagaccccgtgaagggccgatttaccgtctccagagacaacaccaagaacgtggtgtatctgcaagtgaacagcctgaaacctgaggacacggccaattatgtctgtgcaagcagattctcccttatgtcgatcgatgcgagcatgtgcctttcggcgcctcagtatgaccgctggggccaggggacccaggtcagaatctcctcagaacccaagacaccaaaaccacaa

SEQ ID NO: 29 - protein, 136 aa, synthetic construct
  REGION 1..136: note = Description of Artificial Sequence: Synthetic polypeptide
QVQLVETGGLVQPGGSLRLSCTASGFTLGHHRVGWFRQAPGKEREGVACISATGLSSHYSDFVIGRFTVSRDNDNNVVYLQVNGLKPEDTAVYYCASRFSLNSVDANMCLSEPQYDNWGQGTPVRISSEPKTPKPQ

SEQ ID NO: 30 - DNA, 408 bp, synthetic construct
  misc_feature 1..408: note = Description of Artificial Sequence: Synthetic polynucleotide
  source 1..408: mol_type = other DNA
caggtgcagctggtggagacgggtggcttggtgcagcctggggggtctctgagactctcctgtacagcctctggattcactttgggacaccatcgcgttggctggttccgccaggccccaggaaaggagcgtgagggggtcgcgtgtattagcgccactggtcttagttcacactattcagacttcgtgatcggccgatttaccgtctccagagacaacgacaacaacgtggtgtatctacaagtgaacggcctgaaacctgaggacacagccgtttattactgtgcaagcagattctcccttaattcggtcgatgcgaatatgtgcctttcggagcctcagtatgacaactggggccaggggaccccggtcagaatctcctcagaacccaagacaccaaaaccacaa

SEQ ID NO: 31 - protein, 134 aa, synthetic construct
  REGION 1..134: note = Description of Artificial Sequence: Synthetic polypeptide
QVQLVESGGGLVQAGGSLRLSCAASEFRAEHFAVGWFRQAPGKEREGVSCVDASGDSTAYADSVKGRFTISRDNNKNVVYLQMDSLEPEDTGDYYCGASYFTVCAKSMRKIEYRYWGQGTQVTVSSEPKTPKPQ

SEQ ID NO: 32 - DNA, 402 bp, synthetic construct
  misc_feature 1..402: note = Description of Artificial Sequence: Synthetic polynucleotide
  source 1..402: mol_type = other DNA
caggtgcagctggtggagtccggtggaggcttggtgcaggctggggggtctctgagactctcctgtgcagcctctgaattccgtgcggagcattttgccgtgggctggttccgccaggccccagggaaggagcgtgagggggtctcatgtgtagacgcgagtggtgatagtacagcatatgcggactctgtgaagggccgattcaccatctccagagacaacaacaagaacgtagtgtatctgcaaatggacagcctggaacctgaagacacaggagattattattgtggagcctcgtactttactgtctgcgccaagagcatgcggaaaattgaatataggtactggggccaggggacccaggtcaccgtctcctcagaacccaagacaccaaaaccacaa

SEQ ID NO: 33 - protein, 134 aa, synthetic construct
  REGION 1..134: note = Description of Artificial Sequence: Synthetic polypeptide
QVQLAESGGGLVQPGGSLRLSCAASGRALNYYVIGWFRQAPGKEREGVSCIASSEAYTDYADSVQGRFTISRDKALNTVYLDMKRLKPDDTAVYYCAARLRDPNWCGRNADEYDSWGQGTQVTVSSEPKTPKPQ

SEQ ID NO: 34 - DNA, 402 bp, synthetic construct
  misc_feature 1..402: note = Description of Artificial Sequence: Synthetic polynucleotide
  source 1..402: mol_type = other DNA
caggtgcagctcgcggagtcaggcggaggcttggtgcagcctggggggtctctgagactctcctgtgcagcctctggacgcgctttgaattattatgtcataggctggttccgccaggccccagggaaggagcgtgagggggtctcatgtattgcgagtagcgaagcctacacagactatgcagactccgtgcaaggccgattcaccatctcgagagacaaggctctgaatacggtgtatttggatatgaagcgcctgaaacctgacgacacagccgtttattattgtgcagcccggttgcgtgatcctaattggtgcgggcggaatgcggatgagtatgactcctggggccaggggacccaggtcaccgtctcctcagaacccaagacaccaaaaccacaa

SEQ ID NO: 35 - protein, 127 aa, synthetic construct
  REGION 1..127: note = Description of Artificial Sequence: Synthetic polypeptide
QVQLVESGGGLVQAGGSLRLSCAASGFPFGSYYMSWVRQAPGKGPEWVSDISNGGIITRYSDSVKGRFTISRDNAKNILYLQMNSLKPEDTALYFCATGTGRDWSREYRGQGTQVTVSSEPKTPKPQ

SEQ ID NO: 36 - DNA, 381 bp, synthetic construct
  misc_feature 1..381: note = Description of Artificial Sequence: Synthetic polynucleotide
  source 1..381: mol_type = other DNA
caggtgcagctggtggagtccggtggaggcttggtgcaggctggggggtctctgagactctcctgtgcagcctctggattccccttcggtagttactacatgagctgggtccgccaggctccaggaaaggggcccgagtgggtctcagatattagcaatggtggtattattacaaggtattcagactccgtgaagggccgattcaccatctcccgagacaacgccaagaacatattgtatctgcaaatgaacagcctgaaacctgaagacacggccctgtatttctgtgcgacagggaccggtagagactggagcagggagtaccggggccaggggacccaggtcaccgtctcctcagaacccaagacaccaaaaccacaa

SEQ ID NO: 37 - protein, 134 aa, synthetic construct
  REGION 1..134: note = Description of Artificial Sequence: Synthetic polypeptide
QVQLAESGGGLVQPGGSLRLSCEASGFHLEHFAVGWFRQAPGKEREGVSCISASGDSTTYADSVKGRSTISKDNAKNAVYLQMDSLRPEDTGDYYCAASHFSVCGKNIRKIEYRYWGQGTPVTVSSEPKTPKPQ

SEQ ID NO: 38 - DNA, 402 bp, synthetic construct
  misc_feature 1..402: note = Description of Artificial Sequence: Synthetic polynucleotide
  source 1..402: mol_type = other DNA
caggtgcagctcgcggagtctggtggaggcttggtgcagcctggggggtctctgagactctcctgtgaagcctcaggattccatttggagcattttgccgtaggctggttccgccaggccccagggaaggagcgtgagggggtctcatgtataagcgcgagtggtgatagtacaacgtatgcagactccgtgaagggccgatccaccatctccaaagacaacgccaagaacgcggtgtatctgcaaatggacagcctgagacccgaggacacaggcgattattactgtgcagcctcgcacttcagtgtctgcggcaagaacattcggaaaattgagtataggtactggggccaggggaccccggtcaccgtctcctcagaacccaagacaccaaaaccacaa

SEQ ID NO: 39 - protein, 123 aa, synthetic construct
  REGION 1..123: note = Description of Artificial Sequence: Synthetic polypeptide
QVQLVETGGGLVQPGGSLRLSCVVSGLTFNSNYMSWVRQAPGKGPELVSYINSEDGSTFYADSVKGRFTISRDNNENTLYLQMSSLKPEDTARYYCALGIAGATRGQGTQVTVSSEPKTPKPQ

SEQ ID NO: 40 - DNA, 369 bp, synthetic construct
  misc_feature 1..369: note = Description of Artificial Sequence: Synthetic polynucleotide
  source 1..369: mol_type = other DNA
caggtgcagctcgtggagaccgggggaggcttggtgcagccgggggggtctctgagactctcctgtgtagtgtctggattaaccttcaatagcaactacatgagttgggtccgccaggctccagggaaggggcccgagttggtctcatatattaattctgaagatggtagtaccttttatgcagactccgtgaagggccgattcaccatctcgcgagacaacaacgagaatacactgtatctgcaaatgagcagcctgaagcctgaggacacggcccgctattactgtgcactggggatcgctggtgcaactcggggccaggggacccaggtcaccgtctcctcagaacccaagacaccaaaaccacaa

SEQ ID NO: 41 - protein, 137 aa, synthetic construct
  REGION 1..137: note = Description of Artificial Sequence: Synthetic polypeptide
QVQLVESGGGLVQPGGSLRLSCAASGFTLDSYAIGWFRQAPGKEREGVACISASGSGTDYVDSVKGRFTVSRDQAKSMVFLQMNNMKPEDAAVYYCAADYRPRPLPIQAPCTMTGGNYWGQGTQVTVSSEPKTPKPQ

SEQ ID NO: 42 - DNA, 411 bp, synthetic construct
  misc_feature 1..411: note = Description of Artificial Sequence: Synthetic polynucleotide
  source 1..411: mol_type = other DNA
caggtgcagctcgtggagtcagggggaggcttggtgcagcctggggggtctctgagactctcctgtgcagcctctggattcactttagatagttatgcaataggctggttccgccaggccccagggaaggagcgtgagggggtcgcatgtattagtgctagtggtagtggcacggactatgtagactccgtgaagggccgattcaccgtctccagagaccaggccaagagcatggtgtttctgcaaatgaacaacatgaaacctgaggacgcagccgtttattactgtgcagcagattatcggccgaggcccctgccgattcaggcgccgtgtacaatgacaggtggcaactactggggccaggggacccaggtcaccgtctcctcagaacccaagacaccaaaaccacaa

SEQ ID NO: 43 - protein, 138 aa, synthetic construct
  REGION 1..138: note = Description of Artificial Sequence: Synthetic polypeptide
QVQLVESGGGLVQPGGSLTLSCVASGSNLDYFAIGWFRQAPGKEREGVSCISTSSDMSKYADSVKGRFTISRDNTRNTVYLQMNSLEPEDTAVYYCAAKRRRYGLDRDMCLMDSVGMDVWGKGTLVTVSSAHHSEDPS

SEQ ID NO: 44 - DNA, 414 bp, synthetic construct
  misc_feature 1..414: note = Description of Artificial Sequence: Synthetic polynucleotide
  source 1..414: mol_type = other DNA
caggtgcagctggtggagtcggggggaggcttggtgcagcctggggggtctctgacactctcctgtgtagcctctggatccaatttggattattttgcgataggctggttccgccaggccccagggaaggagcgtgagggggtctcatgtattagtacgagtagtgacatgtcaaagtatgcagactccgtgaagggccgcttcaccatctccagagacaacaccaggaacacggtgtatctgcaaatgaacagcctggaacccgaagatacggccgtttattattgtgcagcaaagcgccgccgatatggtctcgatcgtgatatgtgtcttatggattcggtcggcatggacgtgtggggcaaagggaccctggtcaccgtctcctcggcgcaccacagcgaagacccctcg

SEQ ID NO: 45 - protein, 139 aa, synthetic construct
  REGION 1..139: note = Description of Artificial Sequence: Synthetic polypeptide
QVQLVESGGGLVQPGGSLRLSCAAPGFTLDYYAIGWFRQAPGKEREGVSCIRSRGDRTNYADSVKGRFTVSRDNAKNTAYLQMNNLKPEDTGVYFCAAAPRTTVQDLCVTPLLGGADWVSWGQGTQVTVSSEPKTPKPQ

SEQ ID NO: 46 - DNA, 417 bp, synthetic construct
  misc_feature 1..417: note = Description of Artificial Sequence: Synthetic polynucleotide
  source 1..417: mol_type = other DNA
caggtgcagctcgtggagtcaggaggaggcttggtgcagcctggggggtctctgagactctcctgtgcagctcctggattcactttggattattatgccataggctggttccgccaggccccagggaaggagcgtgagggggtctcatgtattcgtagtcgtggtgatcggacaaattatgcagactccgtgaagggccgattcaccgtctccagagacaacgccaagaacacggcgtatctgcaaatgaacaacctgaaacctgaggacacaggcgtttatttctgtgcagctgctccgaggactactgttcaggatttgtgtgtaacccctcttttggggggtgctgactgggtttcctggggccaggggacccaggtcaccgtctcctcggaacccaagacaccaaaaccacaa

SEQ ID NO: 47 - protein, 134 aa, synthetic construct
  REGION 1..134: note = Description of Artificial Sequence: Synthetic polypeptide
QLQLVESGGGLVQPGGSLRLSCAASGFPLGDYTVGWFRQAPGKEREGVSCISKGSRGLRYGDSVKGRFTVARDNAKSTVTLQMDSLKPEDTAVYSCAAGPAMFNQCHMVDNYFTYWGQGTQVTVSSAHHSEDPS

SEQ ID NO: 48 - DNA, 402 bp, synthetic construct
  misc_feature 1..402: note = Description of Artificial Sequence: Synthetic polynucleotide
  source 1..402: mol_type = other DNA
cagttgcagctggtggagtctggcggaggcttggtgcagcctggggggtctctgagactctcctgtgcagcctctggattccctttgggtgattataccgtgggctggttccgccaggccccagggaaggagcgtgagggggtctcatgtattagtaaaggtagtagaggcttaagatacggagactccgtgaaaggccgattcaccgttgccagagacaacgccaagagcacggtaactctgcaaatggacagcctgaaaccggaggacacagccgtttattcttgtgctgcagggccggccatgttcaatcaatgtcatatggtcgacaattactttacatactggggtcaggggacccaggtcaccgtctcctcggcgcaccacagcgaagacccctcg

SEQ ID NO: 49 - protein, 132 aa, synthetic construct
  REGION 1..132: note = Description of Artificial Sequence: Synthetic polypeptide
QVQLVETGGGLVQAGGSLRLSCAASGRTFSNYAMGWFRQAPGKEREFVAAISWSGAHTYYADSVKGRFTISRDNAKSTMYLQMNSLKPEDTAVYYCNADLERYSDFGREVDDYWGQGTQVTVSSEPKTPKPQ

SEQ ID NO: 50 - DNA, 396 bp, synthetic construct
  misc_feature 1..396: note = Description of Artificial Sequence: Synthetic polynucleotide
  source 1..396: mol_type = other DNA
caggtgcagctcgtggagacaggtggaggattggtgcaggctggggggtctctgagactctcctgtgcagcctctggacgcaccttcagtaactatgccatgggctggttccgccaggctccagggaaggagcgtgagtttgtcgcagctattagctggagtggtgctcacacatactatgcagactccgtgaagggccgattcaccatctccagagacaacgccaagagcacgatgtatctgcaaatgaacagcctgaaacctgaggacacggccgtctattactgtaatgcagatctcgagcggtatagtgacttcggtagggaggtggatgactactggggccaggggacccaggtcaccgtctcctcagaacccaagacaccaaaaccacaa

SEQ ID NO: 51 - protein, 137 aa, synthetic construct
  REGION 1..137: note = Description of Artificial Sequence: Synthetic polypeptide
QVQLVESGGGLVQPGGSLRLSCTASGLTLAKWTINWFRQAPGKEREGISCISSSSGSTYYADSVKGRFTISRDNAENTVYLQMSSLKPEDTAVYYCAADSFKGCTFLSSTTHYNNMDYWGKGTLVTVSSAHHSEDPS

SEQ ID NO: 52 - DNA, 411 bp, synthetic construct
  misc_feature 1..411: note = Description of Artificial Sequence: Synthetic polynucleotide
  source 1..411: mol_type = other DNA
caggtgcagctcgtggagtcgggtggaggcttggtgcagcctggggggtctctgagactctcgtgtacagcctctggattaactttggctaagtggaccatcaactggttccgccaggccccagggaaggagcgcgaggggatctcatgtattagtagcagtagtggtagcacatactatgcagactccgtgaagggccgattcaccatctccagagacaacgccgaaaacacggtatatctgcaaatgagcagcctgaaacctgaggacacggccgtttattactgtgcagcggattcttttaagggctgtacgttcctcagtagtactacccattacaacaacatggactactggggcaaagggaccctggtcaccgtctcctcagcgcaccacagcgaagacccctcg

SEQ ID NO: 53 - protein, 132 aa, synthetic construct
  REGION 1..132: note = Description of Artificial Sequence: Synthetic polypeptide
QVQLVESGGGLVQSGGSLRLSCAASRRTASNYAVAWFRQAPGKEREFVAAIGWSDDVTYYADSVKGRFTVSRDNAKNTVYLQMNGLEPEDTAVYYCTTNGDRYSYRTASSYHYWGQGTQVTVSSAHHSEDPS

SEQ ID NO: 54 - DNA, 396 bp, synthetic construct
  misc_feature 1..396: note = Description of Artificial Sequence: Synthetic polynucleotide
  source 1..396: mol_type = other DNA
caggtgcagctcgtggagtcgggtgggggattggtgcagtctgggggctctctgagactctcctgtgcagcctctagacgcaccgccagtaactatgccgtggcctggttccgccaggctccaggaaaggagcgtgagtttgtagcagcgattggctggagtgatgatgtcacgtattacgcagactccgtgaagggccgattcaccgtctccagagacaacgccaagaacacggtgtatctgcaaatgaacggcctggaacctgaggacacggccgtttattactgtacaacaaatggtgatagatacagttacaggacggcatccagctatcactactggggccaggggacccaggtcaccgtctcctcagcgcaccacagcgaagacccctcg

SEQ ID NO: 55 - protein, 132 aa, synthetic construct
  REGION 1..132: note = Description of Artificial Sequence: Synthetic polypeptide
QVQLAETGGGSVQTGGSLRLSCAASGLPFRNYAMAWFRQAPGKEREFVAAISREGGRTYYADFVKGRFTISRDNGRNTIYLEMNSLASEDTAIYYCAGVEGAYTYRTGASYTYWGQGTQVTVSSEPKTPKPQ

SEQ ID NO: 56 - DNA, 396 bp, synthetic construct
  misc_feature 1..396: note = Description of Artificial Sequence: Synthetic polynucleotide
  source 1..396: mol_type = other DNA
caggtgcagctcgcggagactgggggaggatcggtgcagactgggggctctctgaggctctcctgtgcagcctctggactgcccttcagaaactatgccatggcctggttccgccaggctccagggaaggagcgtgagtttgtagcagctattagtcgggaaggcgggaggacatactatgcagacttcgtgaagggccgattcaccatctccagagacaacggcaggaacacgatatatctggagatgaacagcctggcatcggaggatacggccatttattactgtgccggtgtcgagggtgcttatacttatcgtaccggggcctcgtatacttactggggccaggggacccaggtcaccgtctcctcagaacccaagacaccaaaaccacaa

SEQ ID NO: 57 - protein, 131 aa, synthetic construct
  REGION 1..131: note = Description of Artificial Sequence: Synthetic polypeptide
QVQLVETGGGLVQAGGSLRLSCAASGRSYAMGWFRQGPGKEREFVATISWSSTNTWYADSVKGRFTISRDNAKNTVYLQMNSLKPEDTAVYYCAASHRFSDYPMRSEDGMDYWGKGTLVTVSSEPKTPKPQ

SEQ ID NO: 58 - DNA, 393 bp, synthetic construct
  misc_feature 1..393: note = Description of Artificial Sequence: Synthetic polynucleotide
  source 1..393: mol_type = other DNA
caggtgcagctggtggagacggggggaggattggtgcaggctgggggctctctgagactctcctgtgcagcctctggacgcagttatgccatgggctggttccgccagggtccagggaaggagcgtgagtttgtagccactatcagttggagtagtactaacacatggtatgcagattccgtgaagggccgattcaccatctctagagacaacgccaagaacacggtgtatctgcaaatgaacagcctgaaacctgaggacacggctgtttattactgtgcagcgagccatcgttttagcgactatcccatgaggtcagaggacggcatggactactggggcaaagggaccctggtcaccgtctcctcagaacccaagacaccaaaaccacaa

SEQ ID NO: 59 - protein, 132 aa, synthetic construct
  REGION 1..132: note = Description of Artificial Sequence: Synthetic polypeptide
QVQLVETGGGLVQAGGSLRLSCAASGRTFSSYSMGWFRQAPGKEREYVAAVNSNGDSTFYADSIKGRFTVSRDAAKNTVYLQMNSLKPEDTALYYCAAVYGRYTYQSPKSYEYWGQGTQVTVSSEPKTPKPQ

SEQ ID NO: 60 - DNA, 396 bp, synthetic construct
  misc_feature 1..396: note = Description of Artificial Sequence: Synthetic polynucleotide
  source 1..396: mol_type = other DNA
caggtgcagctggtggagacgggaggaggattggtgcaggctgggggctctctgagactctcgtgtgcagcctctggacgcaccttcagtagctattccatgggctggttccgccaggctccagggaaggagcgtgagtatgtagcagcagttaactccaatggcgacagtacattctatgccgactccattaagggccgattcaccgtctccagagacgccgccaagaacacagtctatctgcaaatgaacagcctgaaacctgaggacacggccctttattactgtgcagctgtctacggtagatacacttaccagtccccaaaatcgtatgagtactggggccaggggacccaggtcaccgtctcctcagaacccaagacaccaaaaccacaa

SEQ ID NO: 61 - protein, 137 aa, synthetic construct
  REGION 1..137: note = Description of Artificial Sequence: Synthetic polypeptide
QLQLVETGGGLVKPGGSLRLSCVVSGFTFDDYRMAWVRQAPGKELEWVSSIDSWSINTYYEDSVKGRFTISTDNAKNTLYLQMSSLKPEDTAVYYCAAEDRLGVPTINAHPSKYDYNYWGQGTQVTVSSEPKTPKPQ

SEQ ID NO: 62 - DNA, 411 bp, synthetic construct
  misc_feature 1..411: note = Description of Artificial Sequence: Synthetic polynucleotide
  source 1..411: mol_type = other DNA
cagttgcagctcgtggagactggtggaggcttggtgaagcctgggggttctctgagactctcctgtgtagtctccggattcacttttgatgattatcgcatggcttgggtccgccaggctccagggaaggagctggagtgggtgtccagtatagatagttggagtatcaacacatactatgaagactccgtgaagggccggttcaccatctccacagacaacgccaagaatacactgtatctgcaaatgagcagcctgaaacctgaggacacggccgtgtattactgtgcagcagaggaccgcttaggtgtaccgactattaacgcccacccttcaaaatatgattataactactgggggcaggggacccaggtcaccgtctcctcagaacccaagacaccaaaaccacaa

SEQ ID NO: 63 - protein, 129 aa, synthetic construct
  REGION 1..129: note = Description of Artificial Sequence: Synthetic polypeptide
QVQLVESGGGLVQAGGSLRLSCAASGRTFTSYAMGWFRQAPGKEREFVASISWRGSYTYYSDSVKGRFTISRDYAENTMYLQMNSLKPEXTGRYYCATLTGDVSVGEYDNRGQGTQVTVSSAHHSEDPS

SEQ ID NO: 64 - DNA, 387 bp, synthetic construct
  misc_feature 1..387: note = Description of Artificial Sequence: Synthetic polynucleotide
  source 1..387: mol_type = other DNA
caggtgcagctggtggagtccggtggaggcttggtgcaggctggggggtctctgagactctcctgtgcagcctctggacgcaccttcactagttatgccatgggctggttccgccaggctccagggaaggagcgtgagtttgtagcgtctattagctggcgcggtagttacacatactattcagactccgtgaagggccgattcaccatctccagagattacgccgagaacacgatgtatctgcaaatgaacagcctgaaacctgaggnnacgggcagatattactgtgcaaccttaaccggcgacgtgagtgtcggcgagtatgacaaccggggccaggggacccaggtcactgtctcctcagcgcaccacagcgaagacccctcg

SEQ ID NO: 65 - protein, 132 aa, synthetic construct
  REGION 1..132: note = Description of Artificial Sequence: Synthetic polypeptide
QVQLVESGGGSVQPGGSLRLSCVASGFTFTNYAMAWVRQVSGKGLEGVAAISSEGFIYIPDSVKGRFTISRDNAKNTVYLQMDNLQSEDTAIYHCAAVDWKRVAAMNSYNMDYWGKGTPVTVSAEPKTPKPQ

SEQ ID NO: 66 - DNA, 396 bp, synthetic construct
  misc_feature 1..396: note = Description of Artificial Sequence: Synthetic polynucleotide
  source 1..396: mol_type = other DNA
caggtgcagctggtggagtcggggggaggctcggtgcagcctggggggtctctgagactctcctgtgtagcctctggattcaccttcactaattacgcgatggcctgggtccgccaggtatcagggaaggggctcgagggtgtggccgctattagtagtgagggtttcatatatatcccagactcagtgaagggccgattcaccatctccagagacaacgccaagaacacggtgtatctacaaatggacaacctccagtctgaggatacggccatatatcactgtgcggcagttgattggaaacgggtcgccgcgatgaacagctacaacatggactactggggaaaagggaccccggtcaccgtctccgcagaacccaagacaccaaaaccacaa

SEQ ID NO: 67 - protein, 127 aa, synthetic construct
  REGION 1..127: note = Description of Artificial Sequence: Synthetic polypeptide
QLQLVESGGGLVQAGGSLRLSCAASGRTFSSYAMGWFRQAPGKEREHVAAISWSGGYTYYANSVKGRFTISRDNAKNTVYLQMNSLKPEDTAVYYCNGVQDHSDSLQNWGQGTQVTVSSEPKTPKPQ

SEQ ID NO: 68 - DNA, 381 bp, synthetic construct
  misc_feature 1..381: note = Description of Artificial Sequence: Synthetic polynucleotide
  source 1..381: mol_type = other DNA
cagttgcagctggtggagtcgggcggaggattggtgcaggctggggggtctctgagactctcctgtgcagcctctggacgcaccttcagtagctatgccatgggctggttccgccaggctccagggaaggaacgtgagcatgtcgcagctattagctggagtggtggttacacatactatgcaaactccgtgaagggccgattcaccatctccagagacaacgccaagaacacggtgtatctgcaaatgaacagcctgaaacctgaggacacggccgtctattactgtaatggagttcaggaccatagcgactcccttcagaactggggccaggggacccaggtcaccgtctcctcagaacccaagacaccaaaaccacaa

SEQ ID NO: 69 - protein, 130 aa, synthetic construct
  REGION 1..130: note = Description of Artificial Sequence: Synthetic polypeptide
QLQLVETGGGLVQAGGSLRLSCAASGRTFSSYAVGWFRQAPGKEREFVAAISWSGSYAYYADSVKGRFTISRDNAKNTVYLQMNSLKPEDTAVYYCNGDLEGYSNHETGDYWGQGTQVTVSSEPKTPKPQ

SEQ ID NO: 70 - DNA, 390 bp, synthetic construct
  misc_feature 1..390: note = Description of Artificial Sequence: Synthetic polynucleotide
  source 1..390: mol_type = other DNA
cagttgcagctggtggagacgggaggaggattggtgcaggctggggggtctctgagactctcctgtgcagcctctggacgcaccttcagtagttatgccgtgggctggttccgccaggctccagggaaggagcgtgagtttgtcgcagctattagctggagtggtagttacgcatactatgcagactccgtgaagggccgattcaccatctccagagacaacgccaagaacacggtgtatctgcaaatgaacagcctgaaacctgaggacacggccgtctattactgtaatggagatcttgagggttatagcaaccatgaaaccggggactactggggccaggggacccaggtcaccgtctcctcagaacccaagacaccaaaaccacaa

SEQ ID NO: 71 - protein, 130 aa, synthetic construct
  REGION 1..130: note = Description of Artificial Sequence: Synthetic polypeptide
QLQLAESGGGLVQAGGSLRLSCAASGRTFSSYAMGWFRQAPGKEREFVAAISWTGGYTYYASSVKGRFTISRDNAKNTMYLQMNSLKPEDTAVYYCNADLESYSEYPESYYWGQGTQVTVSSEPKTPKPQ

SEQ ID NO: 72 - DNA, 390 bp, synthetic construct
  misc_feature 1..390: note = Description of Artificial Sequence: Synthetic polynucleotide
  source 1..390: mol_type = other DNA
cagttgcagctggcggagtcgggaggaggattggtgcaggctggggggtctctgagactctcctgtgcagcctctggacgcaccttcagtagctatgccatgggctggttccgccaggctccagggaaggagcgtgagtttgtcgcagctattagctggactggtggttacacatactatgcaagctccgtgaagggccgattcaccatctccagagacaatgccaaaaacacgatgtatctgcaaatgaacagcctgaaaccggaggacacggccgtctattactgtaatgcagatttagaatcctatagcgagtatcccgagagctactactggggccaggggacccaggtcaccgtctcctcagaacccaagacaccaaaaccacaa

SEQ ID NO: 73 - protein, 124 aa, synthetic construct
  REGION 1..124: note = Description of Artificial Sequence: Synthetic polypeptide
QVQLAESGGGLVQAGGSLTLSCAASGLNFDKYAIGWYRQAPGKEREGVSCISKYYNHRMYSDSVKGRFTVSSNYAKNTVYLQMTNLKPEDTAVYYCAAGCIDPEDWGQGTQVTVSSEPKTPKPQ

SEQ ID NO: 74 - DNA, 372 bp, synthetic construct
  misc_feature 1..372: note = Description of Artificial Sequence: Synthetic polynucleotide
  source 1..372: mol_type = other DNA
caggtgcagctggcggagtcggggggaggcttggtgcaggctggggggtctctgacactctcctgtgcagcctctggcctcaatttcgataaatatgccataggctggtaccgccaggccccagggaaagagcgtgagggggtttcatgtattagtaagtattacaatcatcggatgtatagtgactccgtgaagggccgattcaccgtctccagtaactatgccaagaacacggtgtacctgcaaatgaccaatctgaaaccggaggatacggccgtttattactgtgcggcagggtgtattgacccggaagattggggccaggggacccaggtcaccgtctcctcagaacccaagacaccaaaaccacaa

SEQ ID NO: 75 - protein, 133 aa, synthetic construct
  REGION 1..133: note = Description of Artificial Sequence: Synthetic polypeptide
QVQLVETGGGQVQTGGSLRLSCAASEPTFTPKVVGWFRQAPVKERDFVATITIRTGRTLYADSVKGRFTISGDGANNTVYLQMNGLKPEDTAVYYCAASLPLAIPPTQASAYEYWGLGTQVTVSSEPKTPKPQ

SEQ ID NO: 76 - DNA, 399 bp, synthetic construct
  misc_feature 1..399: note = Description of Artificial Sequence: Synthetic polynucleotide
  source 1..399: mol_type = other DNA
caggtgcagctggtggagaccgggggaggccaggtgcagactgggggatctctgagactctcttgcgcagcctctgaacccaccttcactccgaaagttgtgggctggttccgccaggctccagtgaaggagcgtgactttgtagcaactataacaatccgtaccggtcgcacactctatgcagattccgtgaagggccgattcaccatctccggagacggcgccaacaatacggtgtatctacaaatgaacggcctgaaacctgaggacacggccgtttattactgcgccgcatctcttccgctagcaataccaccgacgcaggcttcggcatatgaatactggggcctggggacccaggtcaccgtctcctcagaacccaagacaccaaaaccacaa

SEQ ID NO: 77 - protein, 128 aa, synthetic construct
  REGION 1..128: note = Description of Artificial Sequence: Synthetic polypeptide
QVQLVETGGGLVQPGGSLRLSCSVSGLHFRFANMGWFRQAPGKQRELVAYITTGDNTNYVDHVKGRFTISRDNAKNTVYLQMNSLKPEDTAVYYCNIVNALGEFNPRNDWGQGTQVTVSSEPKTPKPQ

SEQ ID NO: 78 - DNA, 384 bp, synthetic construct
  misc_feature 1..384: note = Description of Artificial Sequence: Synthetic polynucleotide
  source 1..384: mol_type = other DNA
caggtgcagctggtggagacggggggaggcttggtgcagcctggggggtctctgagactctcctgttcagtctctggcctccacttcaggttcgcgaacatgggatggtttcgccaggctccagggaagcagcgcgagttggtcgcatatattactactggtgataacactaactatgtagaccacgtgaagggccgattcaccatctccagagacaacgccaagaacacggtgtatctgcaaatgaacagcctgaaacctgaagacacggccgtctactactgtaatatagtcaatgcgctgggggagttcaatccccgaaacgactggggccaggggacccaggtcaccgtctcctcagaacccaagacaccaaaaccacaa

SEQ ID NO: 79 - protein, 130 aa, synthetic construct
  REGION 1..130: note = Description of Artificial Sequence: Synthetic polypeptide
QVQLVETGGGWVQAGGSLRLSCAASGRAASGNAMAWFRQAPGKEREFVALISWSGGRPYYANSVKGRFAISRDNATNTVYLQMNRLKPEDTAVYYCAASPTIAILPTPYDYWGQGTQVTVSSEPKTPKPQ

SEQ ID NO: 80 - DNA, 390 bp, synthetic construct
  misc_feature 1..390: note = Description of Artificial Sequence: Synthetic polynucleotide
  source 1..390: mol_type = other DNA
caggtgcagctggtggagacgggtggggggtgggtacaggctgggggctctctgagactctcctgtgcagcctctggacgcgccgccagtggaaatgccatggcctggttccgccaggctccaggaaaggagcgtgagtttgtagcattgattagttggagtggtggtcgcccatactatgcaaactccgtgaagggccgattcgccatctccagagacaacgccacgaatacggtgtatctgcaaatgaacagactgaaacctgaggacacggccgtttattactgtgcagcgtcgcctaccatagcgatactacctactccgtatgactactggggccaggggacccaggtcaccgtctcctcagaacccaagacaccaaaaccacaa

SEQ ID NO: 81 - protein, 135 aa, synthetic construct
  REGION 1..135: note = Description of Artificial Sequence: Synthetic polypeptide
QVQLVETGGGLVQAGASLRLSCAASGRTFSTDHMGWFRQAPQKEREFVAAINAWSGLSIYYADSVKGRFTISRDNDKKTAYLQMNSLKPEDTAVYYCAAKEMGRGWVPQSSDDYDAWGQGTQVTVSSEPKTPKPQ

SEQ ID NO: 82 - DNA, 405 bp, synthetic construct
  misc_feature 1..405: note = Description of Artificial Sequence: Synthetic polynucleotide
  source 1..405: mol_type = other DNA
caggtgcagctggtggagacggggggaggattggtgcaggctggggcctctctgagactctcctgtgcagcctctggacgcaccttcagtaccgatcacatgggctggttccgccaggctccacagaaggagcgtgagtttgtggcagcaataaatgcatggagtggactcagcatttactatgcagactccgtgaagggccgattcaccatctccagagacaacgacaagaaaacggcatatctacaaatgaacagcctgaaacctgaggacacggccgtttattactgtgcagccaaggagatgggtaggggttgggtgccacagagctcagacgactatgacgcctggggccaggggacccaggtcaccgtctcctcagaacccaagacaccaaaaccacaa

SEQ ID NO: 83 - protein, 136 aa, synthetic construct
  REGION 1..136: note = Description of Artificial Sequence: Synthetic polypeptide
QVQLVETGGGLVQAGGSLRLSCAVSGRTFSSYAMAWFRQAPGKERDFVAAISWSGGAPHYEDSVKGRFTISRDNAKNMVYLQMNSLKPDDTAVYYCAAAKAGYYSGSYYVGGGMYDYWGQGTQVTVSSEPKTPKPQ

SEQ ID NO: 84 - DNA, 408 bp, synthetic construct
  misc_feature 1..408: note = Description of Artificial Sequence: Synthetic polynucleotide
  source 1..408: mol_type = other DNA
caggtgcagctggtggagactggaggaggattggtgcaggctgggggctctctgagactctcctgtgcagtctctggacgcaccttcagtagctatgccatggcctggttccgccaggctccagggaaggagcgtgattttgtagcagctattagctggagtggtggtgccccacactatgaagactccgtgaagggccgattcaccatctccagagacaacgccaagaacatggtatatctccaaatgaacagcctgaaacctgacgacacggccgtttactactgtgcagcagcgaaagcaggatactatagtggtagttactacgtgggggggggtatgtatgactactggggccaggggacccaggtcaccgtctcctcagaacccaagacaccaaaaccacaa

SEQ ID NO: 85 - protein, 129 aa, synthetic construct
  REGION 1..129: note = Description of Artificial Sequence: Synthetic polypeptide
QVQLVETGGLVQAGGSLRLSCAASGSIGRVDNMGWYRQTPGKERERVAIITGGGTAIYADTVKGRFTVSRDNAKNTIYLQMNSVKPEDTAVYFCNADISRSIESIVYRSYWGQGTQVTVSSEPKTPKPQ

SEQ ID NO: 86 - DNA, 387 bp, synthetic construct
  misc_feature 1..387: note = Description of Artificial Sequence: Synthetic polynucleotide
  source 1..387: mol_type = other DNA
caggtgcagctggtggagacaggaggcttggtgcaggctggggggtctctgagactctcctgtgcagcctccggaagcatcggcagggtcgataacatgggctggtaccgccaaactccagggaaagagcgcgagcgggtcgcaatcattactggaggcggtaccgcgatctatgcagacaccgtgaagggccgattcaccgtctccagagacaacgccaagaacacaatatatctacaaatgaacagcgtgaaacctgaggacacagccgtctatttctgtaatgccgacatcagtcgtagtattgagtccatcgtctatcgttcctactggggccaggggacccaggtcaccgtctcctcagaacccaagacaccaaaaccacaa

SEQ ID NO: 87 - protein, 127 aa, synthetic construct
  REGION 1..127: note = Description of Artificial Sequence: Synthetic polypeptide
QVQLVETGGGLVQPGGSLRLSCAASGNIFSINAMGWYRQAPGKQRELVAAISNSGSTNYEDSVKGRFTVSRDNAKNTVYLQMNSLKPEDTAVYYCNAFDLVAGTRLGSWGQGTQVTVSSEPKTPKPQ

SEQ ID NO: 88 - DNA, 381 bp, synthetic construct
  misc_feature 1..381: note = Description of Artificial Sequence: Synthetic polynucleotide
  source 1..381: mol_type = other DNA
caggtgcagctggtggagacggggggaggcttggtgcagcctggggggtctctgagactctcctgtgcagcctctggaaacatcttcagtatcaatgccatgggctggtaccgccaggctccagggaagcagcgcgagttggtcgcagctattagtaatagtggtagcacaaactatgaagactccgtgaagggccgattcaccgtctccagagacaacgccaagaacacggtgtatctgcaaatgaacagcctgaaacctgaggacacggccgtctattactgtaatgccttcgatttagtagctggtactaggctggggtcctggggccaggggacccaggtcaccgtctcctcggaacccaagacaccaaaaccacaa

SEQ ID NO: 89 - protein, 135 aa, synthetic construct
  REGION 1..135: note = Description of Artificial Sequence: Synthetic polypeptide
QVQLVESGGGLVQPGGSLRLSCAASEFTLEHAAVGWFRQAPGKEREGVSCISSRDSNTYYADSVKGRFTISRDNAENTVYLQMNSLKPEDTAVYYCATDVPCWDGSNWSLGHEYDYWGQGTQVTVSSEPKTPKPQ

SEQ ID NO: 90 - DNA, 405 bp, synthetic construct
  misc_feature 1..405: note = Description of Artificial Sequence: Synthetic polynucleotide
  source 1..405: mol_type = other DNA
caggtgcagctggtggagtcggggggaggcttggtgcagcctggggggtctctgagactctcctgtgcagcctctgaattcactttggaacatgccgccgtaggctggttccgccaggccccagggaaggagcgcgagggggtctcttgtattagtagtcgtgatagtaacacatactatgcagactccgtgaagggccgattcaccatctccagagacaatgccgaaaacacggtatatctgcaaatgaacagcctgaaacctgaggacacggccgtttattactgtgcgacagatgtcccctgctgggacggtagtaactggtccctcggtcatgagtatgactactggggccaggggacccaggtcaccgtctcctcagaacccaagacaccaaaaccacaa

SEQ ID NO: 91 - protein, 131 aa, synthetic construct
  REGION 1..131: note = Description of Artificial Sequence: Synthetic polypeptide
QVQLVETGGGLVQPGGSLRLSCAASGSISSINAMGWYRQAPGKQRELVAAITIRGNTVYGDSVKGRFTVSRDNAKNTVYLQMNSLKPEDTAVYYCNAKSTPSLYAAGYGVDYWGEGTLVTVSSEPKTPKPQ

SEQ ID NO: 92 - DNA, 393 bp, synthetic construct
  misc_feature 1..393: note = Description of Artificial Sequence: Synthetic polynucleotide
  source 1..393: mol_type = other DNA
caggtgcagctggtggagacggggggaggcttggtgcagcctggggggtctctgagactctcctgtgcagcctctggaagcatctccagtatcaatgccatgggctggtaccgccaggctccagggaagcagcgcgagttggtcgcggctattactattcgtggtaacacagtctatggagactccgtgaagggccgattcaccgtctccagagacaacgccaagaacacggtgtatctgcaaatgaacagcctgaaacctgaggacacggccgtctattactgtaatgccaagtcgaccccgagcttgtacgccgccggctacggcgtggactactggggcgaagggaccctagtcaccgtctcctcagaacccaagacaccaaaaccacaa

SEQ ID NO: 93 - protein, 131 aa, synthetic construct
  REGION 1..131: note = Description of Artificial Sequence: Synthetic polypeptide
QVQLVETGGGLVQAGGSLRLSCAASGNISSINAMAWYRQAPGQQRELVAGITSGGRTQYTDSVKGRFTISRDNAKNTVYLQMESLKPEDTAVYYCNAKSPPSTWATGGGMNYWGKGTLVTVSSEPKTPKPQ

SEQ ID NO: 94 - DNA, 393 bp, synthetic construct
  misc_feature 1..393: note = Description of Artificial Sequence: Synthetic polynucleotide
  source 1..393: mol_type = other DNA
caggtgcagctggtggagacggggggaggcttggtgcaggctggggggtctctgagactctcctgtgcagcctctgggaacatctccagtatcaatgccatggcctggtaccgccaggctccagggcagcagcgcgagctggtcgcagggattactagtggtggcaggacacaatatacagactccgtgaagggccgattcaccatctccagagacaacgccaagaacacggtgtatctgcaaatggagagtctgaaacctgaggacacagccgtctattactgtaatgcaaaaagccctcccagtacctgggccacgggggggggcatgaactactggggcaaagggaccctggtcaccgtctcctcagaacccaagacaccaaaaccacaa

SEQ ID NO: 95 - protein, 123 aa, synthetic construct
  REGION 1..123: note = Description of Artificial Sequence: Synthetic polypeptide
QVQLVETGGALVQAGGSLRLSCAASETSSVSLSWMGWYRQAPGKERELVAGINRDRPKYKESVKGRFTISRDNAQNTVYLQMNSLKPEDTAVYYCNTVPPRGDYWGQGTQVTVSSEPKTPKPQ

SEQ ID NO: 96 - DNA, 369 bp, synthetic construct
  misc_feature 1..369: note = Description of Artificial Sequence: Synthetic polynucleotide
  source 1..369: mol_type = other DNA
caggtgcagctggtggagacaggaggagccttggtgcaggcgggggggtctctgagactctcctgtgcagcctctgagacatcttcagtatcgctatcatggatgggctggtaccgccaggctcctgggaaggagcgcgagttggtcgcaggcattaatcgtgataggccaaagtataaagagtccgtgaagggccgattcaccatctccagagacaacgcccagaatacggtgtatctgcaaatgaacagcctgaaacctgaggacacagccgtctattactgtaatacggttccaccacgcggcgactactggggccaggggacccaggtcaccgtctcctcagaacccaagacaccaaaaccacaa

SEQ ID NO: 97 - protein, 128 aa, synthetic construct
  REGION 1..128: note = Description of Artificial Sequence: Synthetic polypeptide
QVQLVESGGGLVQPGGSLRVSCVASGNISSVAAMAWYRQRPEKRRELVAVITNSGGTAYTDSVRGRFTISRDNVKSTVYLQMNNLKPEDTAVYYCNARGLDAGSGRIDYWGQGTQVTVSSEPKTPKPQ

SEQ ID NO: 98 - DNA, 384 bp, synthetic construct
  misc_feature 1..384: note = Description of Artificial Sequence: Synthetic polynucleotide
  source 1..384: mol_type = other DNA
caggtgcagctggtggagtccggtggaggcttggtgcagcctggggggtctctgagagtctcctgtgtagcctctggaaacatctccagtgtcgctgccatggcctggtaccgccagagaccagagaagcgccgcgaattggtcgcagtcattactaacagcggtggcacagcctatacagactccgtgaggggccgattcaccatctccagagacaatgtcaagtcaacggtgtatctacaaatgaataacctgaaacctgaggacacagccgtgtattactgtaatgcgagggggttagacgccgggtcagggcgcattgactactggggccagggaacccaggtcaccgtctcctcagaacccaagacaccaaaaccacaa

SEQ ID NO: 99 - protein, 130 aa, synthetic construct
  REGION 1..130: note = Description of Artificial Sequence: Synthetic polypeptide
QVQLVESGGGLAQTGGSLNLSCAASGPTFSGYGMGWFRQAPGKEREFLAVIRWSVGNTLYAESVKGRFTISRDKVKNTGYLQIDNLKPEDTAVYYCAAGAYVTTRSRDYAYWGQGTQVTVSSEPKTPKPQ

SEQ ID NO: 100 - DNA, 390 bp, synthetic construct
  misc_feature 1..390: note = Description of Artificial Sequence: Synthetic polynucleotide
  source 1..390: mol_type = other DNA
caggtgcagctggtggagtcggggggaggattggcgcagactgggggctctctgaacctctcctgtgcagcctctggaccgactttcagcggctatggtatgggctggttccgccaggctccagggaaggagcgtgaatttctagcggtaattcgctggagtgtaggtaatacattgtatgcagagtccgtcaagggccgattcaccatctccagagacaaggtcaagaacacggggtatctgcaaatagacaacctgaaacccgaggacacggccgtttattactgtgcagcgggggcgtacgtaactacgaggtcccgcgactatgcctactggggccaggggacccaggtcaccgtctcctcagaacccaagacaccaaaaccacaa

SEQ ID NO: 101 - protein, 133 aa, synthetic construct
  REGION 1..133: note = Description of Artificial Sequence: Synthetic polypeptide
QVQLVETGGRQVQTGDSLNLSCAASEHTFSPKVMGWFRQAPGKGREFVATITIRGGRTLYADSVKGRFAISKDGAKNTVYLQMNSLKPEDTAVYYCAASRELAIPPTQPSAYDHWGQGTQVTVSSAHHSEDPS

SEQ ID NO: 102 - DNA, 399 bp, synthetic construct
  misc_feature 1..399: note = Description of Artificial Sequence: Synthetic polynucleotide
  source 1..399: mol_type = other DNA
caggtgcagctcgtggagaccggcggacgtcaggtgcagactggggactctctgaacctctcttgcgcagcttctgaacacaccttcagtcctaaagttatggggtggttccgccaggctccaggcaaggggcgtgagtttgtagcaactatcacaatccgtggcggtcgcacactctatgcagattccgtgaagggccgatttgccatctccaaagacggcgccaagaatacggtgtatctgcaaatgaacagtctgaaacctgaggacacggccgtttattactgtgcagcaagtcgtgagctagcgataccaccgacgcagccttcggcatacgaccactggggccaggggacccaggtcaccgtctcctcagcgcaccacagcgaagacccctcg

SEQ ID NO: 103 - protein, 274 aa, synthetic construct
  REGION 1..274: note = Description of Artificial Sequence: Synthetic polypeptide
QVQLAESGGGLVQPGGSLGLSCVVASERSINNYGMGWYRQAPGKQRELVAQISSGGTTNYADSVEGRFTISRDNVKKMVHLQVNSLKPEDTAVYYCNSLLRTFSWGQGTQVTVSSEPKTPKPQAIAGGGGSGGGGSGGGGSLQGQVQLVESGGGLVQPGGSLSVSCAASGSIARPGAMAWYRQAPGKERELVASITPGGLTNYADSVTGRFTISRDNAKRTVYLQMNSLQPEDTAVYYCHARIIPLGLGSEYRDHWGQGTQVTVSSAHHSEDPS

SEQ ID NO: 104 - DNA, 822 bp, synthetic construct
  misc_feature 1..822: note = Description of Artificial Sequence: Synthetic polynucleotide
  source 1..822: mol_type = other DNA
caggtgcagctggcggagtcgggcggaggcttggtgcagcctggggggtctctgggactctcctgtgtagtcgcctctgaaagaagcatcaataattatggcatgggctggtaccgccaggctccagggaagcagcgcgagttggtcgcgcaaattagtagtggtggtaccacaaattatgcagactccgtagagggccgattcaccatctccagagacaacgtcaagaaaatggtgcatcttcaagtgaacagcctgaaacctgaggacacggccgtctattactgtaattcgctactccgaactttttcctggggccaggggacccaggtcaccgtctcctcggaacccaagacaccaaaaccacaagcgatcgctggtggaggcggttcaggcggaggtggctctggcggtggcggttccctgcagggtcagktgcagctsgyggagtccgggggcggcttggtgcagcccggggggtctctgagtgtctcctgtgcagcctctggaagcatcgcaagaccaggtgccatggcctggtaccgccaggctccagggaaggagcgcgagttggtcgcgtctattacgcctggtggtcttacaaactatgcggactccgtgacgggccgattcaccatttccagagacaacgccaagaggacggtgtatctgcagatgaacagcctccaacccgaggacacggccgtctattactgtcatgcacgaataattcccctaggacttgggtccgaatacagggaccactggggccaggggactcaggtcaccgtctcctcagcgcaccacagcgaagacccctcg

SEQ ID NO: 105 - protein, 286 aa, synthetic construct
  REGION 1..286: note = Description of Artificial Sequence: Synthetic polypeptide
QVQLAESGGGLVQPGGSLGLSCVVASERSINNYGMGWYRQAPGKQRELVAQISSGGTTNYADSVEGRFTISRDNVKKMVHLQVNSLKPEDTAVYYCNSLLRTFSWGQGTQVTVSSEPKTPKPQAIAGGGGSGGGGSGGGGSLQGQVQLAESGGGGLVQAGGSLRLSCAASGRTFSGYAMGWFRQAPGKEREFVADISWSGHNTYYGDSVKGRFTISRDTAKNTVYLQMNSLKPEDTAVYYCAAEGARTHLSDSYYFPGLWAEPPVGYWGQGTQVTVSSEPKTPKPQ

SEQ ID NO: 106 - DNA, 858 bp, synthetic construct
  misc_feature 1..858: note = Description of Artificial Sequence: Synthetic polynucleotide
  source 1..858: mol_type = other DNA
caggtgcagctggcggagtcgggcggaggcttggtgcagcctggggggtctctgggactctcctgtgtagtcgcctctgaaagaagcatcaataattatggcatgggctggtaccgccaggctccagggaagcagcgcgagttggtcgcgcaaattagtagtggtggtaccacaaattatgcagactccgtagagggccgattcaccatctccagagacaacgtcaagaaaatggtgcatcttcaagtgaacagcctgaaacctgaggacacggccgtctattactgtaattcgctactccgaactttttcctggggccaggggacccaggtcaccgtctcctcggaacccaagacaccaaaaccacaagcgatcgctggtggaggcggttcaggcggaggtggctctggcggtggcggttccctgcagggtcaggtgcagctcgcggagtcgggtgggggaggactggtgcaggctgggggctctctgagactctcctgtgcagcctctggacgcaccttcagtggctatgccatgggctggttccgccaggctccggggaaggagcgtgagtttgtagccgatattagctggagtggtcataacacgtactatggagactccgtgaagggccgattcaccatctccagagacaccgccaagaacacggtgtatctgcaaatgaacagcctgaaacctgaggacacggccgtttattactgtgcagcggagggggcccgtacacaccttagtgatagttactacttcccgggcctctgggccgaaccccccgtgggctactggggccaggggacccaggtcactgtctcctcagaacccaagacaccaaaaccacaa

SEQ ID NO: 107 - protein, 136 aa, synthetic construct
  REGION 1..136: note = Description of Artificial Sequence: Synthetic polypeptide
QVQLVESGGGSVQAGGSLRLTCTGSGRSFALYYMAWFRQAPGKEREFVAAISHNSLSAIVADSLKGRFTISRDNARNQVVLQMNSLKPEDTAVYYCAADFSPSTYNTNYYRTGSYQYWGQGTQVTVSSEPKTPKPQ

SEQ ID NO: 108 - DNA, 408 bp, synthetic construct
  misc_feature 1..408: note = Description of Artificial Sequence: Synthetic polynucleotide
  source 1..408: mol_type = other DNA
caggtgcagctggtggagtcggggggaggatcggtgcaggctgggggctctctgagactcacctgtacaggctctggacgcagtttcgcgctctattacatggcctggttccgccaggctccagggaaggagcgtgagtttgtagcagctatcagccacaattcgttaagcgcaatcgttgcagactccctaaagggccgattcaccatctccagagacaacgccagaaaccaggtggttctacaaatgaacagcctgaaacctgaggacacggccgtttattactgtgcagcagacttttcgccctcgacctataatacaaattactaccgcaccggttcgtatcagtattggggccaggggacccaggtcaccgtctcctcagaacccaagacaccaaaaccacaa

SEQ ID NO: 109 - protein, 137 aa, synthetic construct
  REGION 1..137: note = Description of Artificial Sequence: Synthetic polypeptide
QVQLVETGGGLVQAGGSLGLSCAASGLSFNWYDVGWFRQAPGKEREFVASRSSGGGSTYYGDSVKGRFSISTDNAKNTAYLQMNSLKPEDTAVYYCAADWTGRAGFSVGYYRPDEYDYWGQGTQVTVSEEPKTPKPQ

SEQ ID NO: 110 - DNA, 411 bp, synthetic construct
  misc_feature 1..411: note = Description of Artificial Sequence: Synthetic polynucleotide
  source 1..411: mol_type = other DNA
caggtgcagctggtggagacgggaggaggattggtgcaggctgggggctctctgggactctcctgtgcagcctctggactgtcctttaattggtatgacgtgggctggttccgccaggctccagggaaggagcgtgagtttgtagcgtctcgtagctcgggtggtggtagtacatattatggagactccgtgaagggccgattcagcatctccacagacaatgccaagaacacggcgtatctgcaaatgaacagcctaaaacctgaggacacggccgtttactactgtgcagcagattggacaggccgcgcaggcttcagtgttggttactaccggcccgatgagtatgactactggggccaggggacccaggtcaccgtctccgaagaacccaagacaccaaaaccacaa

SEQ ID NO: 111 - protein, 134 aa, synthetic construct
  REGION 1..134: note = Description of Artificial Sequence: Synthetic polypeptide
QVQLVETGGGLVQPGGSLRLSCVASGFTLDSYAIGWFRQAPGKEREGVSCMSSGDGSTYYTNSVKGRFTISRDNAQNTVYLQMNSLKPEDTAVYYCAADGFDYCSAYVPGRGMNYSGKGTLVTVSSEPKTPKPQ

SEQ ID NO: 112 - DNA, 402 bp, synthetic construct
  misc_feature 1..402: note = Description of Artificial Sequence: Synthetic polynucleotide
  source 1..402: mol_type = other DNA
caggtgcagctggtggagacggggggaggcttggtgcagcctggggggtctctgagactctcctgtgtagcctctggattcactttggattcatatgccataggctggttccgccaggccccagggaaggagcgtgagggggtctcatgtatgagtagtggtgatggtagcacatactatacaaactccgtgaagggccgattcaccatctccagagacaacgcccagaacacggtgtatctgcaaatgaacagcctgaaacctgaggacacagccgtttattactgtgcagcagatgggtttgactattgttcagcttatgtgcccgggagaggcatgaactactcgggcaaagggaccctggtcaccgtctcctcagaacccaagacaccaaaaccacaa

SEQ ID NO: 113 - protein, 135 aa, synthetic construct
  REGION 1..135: note = Description of Artificial Sequence: Synthetic polypeptide
QVQLVETGGGLVQPGGSLRLSCAGSGFTLDNYAVGWFRQAPGKEREGVSCISSSDDNTDYSDSVKGRFTISRDNAKDTVYLQMNSLKPEDTAIYYCAAESPTFGFSCTVATDPYDYWGQGTQVTVSSEPKTPKPQ

SEQ ID NO: 114 - DNA, 405 bp, synthetic construct
  misc_feature 1..405: note = Description of Artificial Sequence: Synthetic polynucleotide
  source 1..405: mol_type = other DNA
caggtgcagctggtggagacgggtggaggcttggtgcagcctggggggtctctgagactctcctgtgcaggctctggattcactttggataattatgccgtcggctggttccgccaggccccagggaaggagcgtgagggggtctcatgtattagtagtagtgatgataacactgactattcagactccgtgaagggccgattcaccatctccagagacaacgccaaggacacggtctatctgcaaatgaacagcctgaaacctgaggacacagcgatttattactgtgcagcagaaagcccgacgttcgggttcagctgtacggtagccactgatccatatgactactggggccaggggacccaggtcaccgtctcctcagaacccaagacaccaaaaccacaa

SEQ ID NO: 115 - protein, 130 aa, synthetic construct
  REGION 1..130: note = Description of Artificial Sequence: Synthetic polypeptide
QVQLVETGGGLVQPGGSLRLSCAASGFTLDGYAAGWFRQAPGKERELVSWISSTDGSTYYAASVKGRFTVSRDNAKNTVYLQMNSLKPEDTAVYYCTAGLGLDVSDYVYDYWGQGTQVTVSSEPKTPKPQ

SEQ ID NO: 116 - DNA, 390 bp, synthetic construct
  misc_feature 1..390: note = Description of Artificial Sequence: Synthetic polynucleotide
  source 1..390: mol_type = other DNA
caggtgcagctggtggagacggggggaggcttggtgcagcctggggggtctctgaggctctcctgtgcagcctctggattcactttggatggctatgccgcaggctggttccgccaggccccagggaaggagcgtgagttggtctcatggattagtagcactgatggtagcacatactatgcagcctccgtgaagggccgattcaccgtctccagagacaacgccaagaacacggtgtatctacaaatgaacagcctgaaacctgaggacacagccgtttattactgtacagcaggtctagggcttgacgttagcgactatgtatatgactactggggccaggggacccaggtcaccgtctcctcagaacccaagacaccaaaaccacaa

SEQ ID NO: 117 - protein, 128 aa, synthetic construct
  REGION 1..128: note = Description of Artificial Sequence: Synthetic polypeptide
QVQLVESGGLVQPGGSLRLSCAASGFTLDYYGIGWVRQAPGKEREEVSCITSGGLTNYPDSVKGRFTISRDNAKNTVYLQMNSLKPEDTAVYYCAIDRVGVCAMEDFGSWGQGTQVTVSSEPKTPKPQ

SEQ ID NO: 118 - DNA, 384 bp, synthetic construct
  misc_feature 1..384: note = Description of Artificial Sequence: Synthetic polynucleotide
  source 1..384: mol_type = other DNA
caggtgcagctggtggagtcgggcggcttggtgcagcctggggggtctctgagactctcctgtgcagcctctggattcactttggattattatggcataggctgggtccgccaggccccagggaaggagcgtgaggaggtctcatgtattactagtggtggtctcacaaactatccagactccgtgaagggccgattcaccatctccagagacaacgccaagaacacagtgtatctgcaaatgaacagcctgaaacctgaggacacggccgtttattactgtgcaatcgaccgtgtgggagtatgcgcgatggaggactttggttcctggggccaggggacccaggtcaccgtctcctcggaacccaagacaccaaaaccacaa

SEQ ID NO: 119 - protein, 132 aa, synthetic construct
  REGION 1..132: note = Description of Artificial Sequence: Synthetic polypeptide
QVQLVETGGGLVQAGDSLRLSCAASGRTFNYYAMAWFRQAPGKEREFVAFINWSGDSTYYAGSVKGRFTISRDNAKNTVYLQMNNLKPEDTAVYSCAAEFGTFSYLQGDDYSYWGQGTQVTVSSEPKTPKPQ

SEQ ID NO: 120 - DNA, 396 bp, synthetic construct
  misc_feature 1..396: note = Description of Artificial Sequence: Synthetic polynucleotide
  source 1..396: mol_type = other DNA
caggtgcagctggtggagacaggtggaggattggtgcaggctggggactctctgagactctcctgtgcagcctctggacgcaccttcaattactatgccatggcctggttccgccaggccccaggaaaggagcgtgaatttgtagcatttattaactggagcggcgatagtacatactatgcaggctccgtgaagggccgattcaccatctccagagacaacgccaagaacacggtgtatctgcaaatgaacaacctgaaacctgaggacacggccgtttattcctgtgcagcagaattcggtacattttcctacttgcaaggcgatgactatagctactggggccaggggacccaggtcaccgtctcctcagaacccaagacaccaaaaccacaa

SEQ ID NO: 121 - protein, 131 aa, synthetic construct
  REGION 1..131: note = Description of Artificial Sequence: Synthetic polypeptide
QVQLVESGGGLVQAGGSLRLSCAASGRSFSSYRMGWFRQAPGKERELVAGISWSGSSTWYADSVKGRFTISRDNAKNTVYLQMNSLKPEDTAVYYCAADGLGTDWSDAIWDYWGQGTQVTVSSEPKTPKPQ

SEQ ID NO: 122 - DNA, 393 bp, synthetic construct
  misc_feature 1..393: note = Description of Artificial Sequence: Synthetic polynucleotide
  source 1..393: mol_type = other DNA
caggtgcagctggtggagtctggaggaggattggtgcaggctgggggctctctgagactctcctgtgcagcctctggacgcagcttcagtagctatcgcatgggctggttccgccaggctccagggaaggagcgtgagcttgtagcaggtattagctggagtggaagtagtacatggtatgcagactccgtgaagggccgattcaccatctccagagacaacgccaagaacacggtgtatctgcaaatgaacagcctgaaacccgaggacacggccgtttattactgtgcagcagatgggctagggacggattggagcgatgccatatgggactactggggccaggggacccaggtcaccgtctcctcagaacccaagacaccaaaaccacaa

SEQ ID NO: 123 - protein, 135 aa, synthetic construct
  REGION 1..135: note = Description of Artificial Sequence: Synthetic polypeptide
QVQLVESGGGLVQAGGSLRLSCAASGRNFSHYAMGWFRQAPGKAREFVATINRDGDSTYYTNSVKGRFTISRENAKNTGYLQMNSLKPEDTAVYYCGVQYSWSGTSIYWREYEYAYWGQGAQVTVSSEPKTPKPQ

SEQ ID NO: 124 - DNA, 405 bp, synthetic construct
  misc_feature 1..405: note = Description of Artificial Sequence: Synthetic polynucleotide
  source 1..405: mol_type = other DNA
caggtgcagctggtggagtcggggggaggattggtgcaggctgggggctctctgagactctcctgtgcagcctctggacgcaatttcagtcactatgccatgggctggttccgccaggctccagggaaggcgcgtgagtttgtagcaactattaaccgggatggtgatagcacatactatacgaactccgtgaagggccgattcaccatctccagagagaacgccaagaacacgggatatctgcaaatgaacagcctgaaacctgaggacacggccgtttattactgtggagtacaatactcgtggtcgggtacaagtatttactggagggagtatgagtatgcctactggggccagggggcccaggtcaccgtctcctcagaacccaagacaccaaaaccacaa

SEQ ID NO: 125 - protein, 126 aa, synthetic construct
  REGION 1..126: note = Description of Artificial Sequence: Synthetic polypeptide
QVQLVESGGGLVQPGGSLRLSCAASGFPFHAYYMSWVRQAPGKGLEWVSHIGNGGIITRYADSVKGRFTISRDNAKNTLYLQMTNLKPEDTALYYCTLGTRDDLGPERGQGTQVTVSSEPKTPKPQ

SEQ ID NO: 126 - DNA, 378 bp, synthetic construct
  misc_feature 1..378: note = Description of Artificial Sequence: Synthetic polynucleotide
  source 1..378: mol_type = other DNA
caggtgcagctggtggagtcgggtggaggcttggtgcagcctggggggtctctgagactctcctgtgcagcctctggattccccttccatgcctactacatgagctgggtccgccaggctccaggaaaggggctcgagtgggtctcccatattggcaatggtggtattattacacgctatgcagactccgtgaagggccggttcaccatctccagagacaacgccaagaacacgctgtatctgcaaatgaccaacctgaaacctgaggacacggccctgtattattgtaccctggggacccgcgacgacctggggcctgagaggggccaggggacccaggtcaccgtctcctcagaacccaagacaccaaaaccacaa

SEQ ID NO: 127 - protein, 123 aa, synthetic construct
  REGION 1..123: note = Description of Artificial Sequence: Synthetic polypeptide
QVQLVESGGGLVQPGGSLRLSCAASEGIFSVDAMGWYRQVPGKQRELVARITRGGSIIYADSVKGRFTISRDSAKNTVYLQMNSLKPEDTAVYYCNRLYRGTLTFGQGTQVTVSSAHHSEDPS

SEQ ID NO: 128 - DNA, 369 bp, synthetic construct
  misc_feature 1..369: note = Description of Artificial Sequence: Synthetic polynucleotide
  source 1..369: mol_type = other DNA
caggtgcagctcgtggagtcgggtggaggcttggtgcagcctggggggtctctgagactctcctgtgcagcctctgagggaatcttcagtgttgatgccatgggctggtaccgccaggttccagggaagcagcgcgagttggtcgcacgaattacccgtggtggtagcataatttatgcagactccgtgaagggccgattcaccatctccagagacagcgccaagaacacggtgtatctgcaaatgaacagcctgaaacctgaggacacggccgtctattactgtaatcgcctttataggggtaccctaacgttcggccaggggacccaggtcaccgtctcctcagcgcaccacagcgaagacccctcg

SEQ ID NO: 129 - protein, 129 aa, synthetic construct
  REGION 1..129: note = Description of Artificial Sequence: Synthetic polypeptide
QVQLVETGGGLVQAGGSLRLSCAASGRTFSIYAMGWFRQAPGREREFVASISRMGWSTYYGDSVKGRFTASRDNAKNTLYLQMNSLELEDTAVYFCAASASALRVNQWDYWGQGTQVTVSSEPKTPKPQ

SEQ ID NO: 130 - DNA, 387 bp, synthetic construct
  misc_feature 1..387: note = Description of Artificial Sequence: Synthetic polynucleotide
  source 1..387: mol_type = other DNA
caggtgcagctggtggagaccggcggaggattggtgcaggctgggggctctctgagactctcctgtgcagcctctggacgcaccttcagtatctatgccatgggctggttccgccaggctccagggagggagcgtgagtttgtagcgtctattagtcggatgggttggagcacatattatggggactccgtgaagggccgattcaccgcctccagagacaacgccaagaacacgctgtatctacaaatgaacagcctcgaacttgaggacacggccgtatatttttgtgcggcatctgcgagtgcgttacgagttaatcagtgggactactggggccaggggacccaggtcaccgtctcctcagaacccaagacaccaaaaccacaa

SEQ ID NO: 131 - protein, 293 aa, synthetic construct
  REGION 1..293: note = Description of Artificial Sequence: Synthetic polypeptide
QVQLVESGGGLVQPGGSLRLSCAASGFTLDSYAIGWFRQAPGKEREGVACISASGSGTDYVDSVKGRFTVSRDQAKSMVFLQMNNMKPEDAAVYYCAADYRPRPLPIQAPCTMTGGNYWGQGTQVTVSSEPKTPKPQAIAGGGGSGGGGSGGGGSLQGQVQLVESGGGLVQAGGSLRLSCAASILTYDLDYYYIGWVRQAPGKEREGVSCISSTDGATYYADSVKGRFTISRNNAKNTVYLQMNNLKPEDTAIYYCAAAPLAGRYCPASHEYGYWGQGTQVTVSSAHHSEDPS

SEQ ID NO: 132 - DNA, 879 bp, synthetic construct
  misc_feature 1..879: note = Description of Artificial Sequence: Synthetic polynucleotide
  source 1..879: mol_type = other DNA
caggtgcagctcgtggagtcagggggaggcttggtgcagcctggggggtctctgagactctcctgtgcagcctctggattcactttagatagttatgcaataggctggttccgccaggccccagggaaggagcgtgagggggtcgcatgtattagtgctagtggtagtggcacggactatgtagactccgtgaagggccgattcaccgtctccagagaccaggccaagagcatggtgtttctgcaaatgaacaacatgaaacctgaggacgcagccgtttattactgtgcagcagattatcggccgaggcccctgccgattcaggcgccgtgtacaatgacaggtggcaactactggggccaggggacccaggtcaccgtctcctcagaacccaagacaccaaaaccacaagcgatcgctggtggaggcggttcaggcggaggtggctctggcggtggcggttccctgcagggtcaggtgcagctcgtggagtccggtggaggcttggtgcaggctggggggtctctgagactctcctgtgcagcctctatactcacttatgatttggattattattacataggctgggtccgccaggccccagggaaggagcgtgagggggtctcatgtattagtagtactgatggtgccacatactatgcagactccgtgaagggccgattcaccatctccagaaacaacgccaagaacacggtgtatctgcaaatgaacaacctaaaacctgaggacacagccatttattattgtgcagcagcccccctggctgggcgctactgtcccgcctcgcatgagtatggctactggggtcaggggacccaggtcaccgtctcgtcagcgcaccacagcgaagacccctcg

SEQ ID NO: 133 - protein, 292 aa, synthetic construct
  REGION 1..292: note = Description of Artificial Sequence: Synthetic polypeptide
QVQLVESGGGLVQPGGSLRLSCAASGFTLDSYAIGWFRQAPGKEREGVACISASGSGTDYVDSVKGRFTVSRDQAKSMVFLQMNNMKPEDAAVYYCAADYRPRPLPIQAPCTMTGGNYWGQGTQVTVSSEPKTPKPQAIAGGGGSGGGGSGGGGSLQGQXQLXESGGGLVQAGGSLRLSCAASEFRAEHFAVGWFRQAPGKEREGVSCVDASGDSTAYADSVKGRFTISRDNNKNVVYLQMDSLEPEDTGDYYCGASYFTVCAKSMRKIEYRYWGQGTQVTVSSEPKTPKPQ

SEQ ID NO: 134 - DNA, 876 bp, synthetic construct
  misc_feature 1..876: note = Description of Artificial Sequence: Synthetic polynucleotide
  source 1..876: mol_type = other DNA
caggtgcagctcgtggagtcagggggaggcttggtgcagcctggggggtctctgagactctcctgtgcagcctctggattcactttagatagttatgcaataggctggttccgccaggccccagggaaggagcgtgagggggtcgcatgtattagtgctagtggtagtggcacggactatgtagactccgtgaagggccgattcaccgtctccagagaccaggccaagagcatggtgtttctgcaaatgaacaacatgaaacctgaggacgcagccgtttattactgtgcagcagattatcggccgaggcccctgccgattcaggcgccgtgtacaatgacaggtggcaactactggggccaggggacccaggtcaccgtctcctcagaacccaagacaccaaaaccacaagcgatcgctggtggaggcggttcaggcggaggtggctctggcggtggcggttccctgcagggtcagktgcagctsgyggagtccggtggaggcttggtgcaggctggggggtctctgagactctcctgtgcagcctctgaattccgtgcggagcattttgccgtgggctggttccgccaggccccagggaaggagcgtgagggggtctcatgtgtagacgcgagtggtgatagtacagcatatgcggactctgtgaagggccgattcaccatctccagagacaacaacaagaacgtagtgtatctgcaaatggacagcctggaacctgaagacacaggagattattattgtggagcctcgtactttactgtctgcgccaagagcatgcggaaaattgaatataggtactggggccaggggacccaggtcaccgtctcctcagaacccaagacaccaaaaccacaa

SEQ ID NO: 135 - protein, 290 aa, synthetic construct
  REGION 1..290: note = Description of Artificial Sequence: Synthetic polypeptide
QVQLVESGGGLVQAGGSLRLSCAASILTYDLDYYYIGWVRQAPGKEREGVSCISSTDGATYYADSVKGRFTISRNNAKNTVYLQMNNLKPEDTAIYYCAAAPLAGRYCPASHEYGYWGQGTQVTVSSAHHSEDPSAIAGGGGSGGGGSGGGGSLQGQVQLVESGGGLVQPGGSLRLSCEASGFHLEHFAVGWFRQAPGKEREGVSCISASGDSTTYADSVKGRSTISKDNAKNAVYLQMDSLRPEDTGDYYCAASHFSVCGKNIRKIEYRYWGQGTPVTVSSEPKTPKPQ

SEQ ID NO: 136 - DNA, 870 bp, synthetic construct
  misc_feature 1..870: note = Description of Artificial Sequence: Synthetic polynucleotide
  source 1..870: mol_type = other DNA
caggtgcagctggtggagtccggtggaggcttggtgcaggctggggggtctctgagactctcctgtgcagcctctatactcacttatgatttggattattattacataggctgggtccgccaggccccagggaaggagcgtgagggggtctcatgtattagtagtactgatggtgccacatactatgcagactccgtgaagggccgattcaccatctccagaaacaacgccaagaacacggtgtatctgcaaatgaacaacctaaaacctgaggacacagccatttattattgtgcagcagcccccctggctgggcgctactgtcccgcctcgcatgagtatggctactggggtcaggggacccaggtcaccgtctcgtcagcgcaccacagcgaagacccctcggcgatcgctggtggaggcggttcaggcggaggtggctctggcggtggcggttccctgcagggtcaggtgcagctggtggagtctggtggaggcttggtgcagcctggggggtctctgagactctcctgtgaagcctcaggattccatttggagcattttgccgtaggctggttccgccaggccccagggaaggagcgtgagggggtctcatgtataagcgcgagtggtgatagtacaacgtatgcagactccgtgaagggccgatccaccatctccaaagacaacgccaagaacgcggtgtatctgcaaatggacagcctgagacccgaggacacaggcgattattactgtgcagcctcgcacttcagtgtctgcggcaagaacattcggaaaattgagtataggtactggggccaggggaccccggtcaccgtctcctcagaacccaagacaccaaaaccacaa

SEQ ID NO: 137 - protein, 290 aa, synthetic construct
  REGION 1..290: note = Description of Artificial Sequence: Synthetic polypeptide
QVQLVESGGGLVQAGGSLRLSCAASILTYDLDYYYIGWVRQAPGKEREGVSCISSTDGATYYADSVKGRFTISRNNAKNTVYLQMNNLKPEDTAIYYCAAAPLAGRYCPASHEYGYWGQGTQVTVSSAHHSEDPSAIAGGGGSGGGGSGGGGSLQGQVQLAESGGGLVQAGGSLRLSCAASEFRAEHFAVGWFRQAPGKEREGVSCVDASGDSTAYADSVKGRFTISRDNNKNVVYLQMDSLEPEDTGDYYCGASYFTVCAKSMRKIEYRYWGQGTQVTVSSEPKTPKPQ

SEQ ID NO: 138 - DNA, 870 bp, synthetic construct
  misc_feature 1..870: note = Description of Artificial Sequence: Synthetic polynucleotide
  source 1..870: mol_type = other DNA
caggtgcagctggtggagtccggtggaggcttggtgcaggctggggggtctctgagactctcctgtgcagcctctatactcacttatgatttggattattattacataggctgggtccgccaggccccagggaaggagcgtgagggggtctcatgtattagtagtactgatggtgccacatactatgcagactccgtgaagggccgattcaccatctccagaaacaacgccaagaacacggtgtatctgcaaatgaacaacctaaaacctgaggacacagccatttattattgtgcagcagcccccctggctgggcgctactgtcccgcctcgcatgagtatggctactggggtcaggggacccaggtcaccgtctcgtcagcgcaccacagcgaagacccctcggcgatcgctggtggaggcggttcaggcggaggtggctctggcggtggcggttccctgcagggtcaggtgcagctggcggagtccggtggaggcttggtgcaggctggggggtctctgagactctcctgtgcagcctctgaattccgtgcggagcattttgccgtgggctggttccgccaggccccagggaaggagcgtgagggggtctcatgtgtagacgcgagtggtgatagtacagcatatgcggactctgtgaagggccgattcaccatctccagagacaacaacaagaacgtagtgtatctgcaaatggacagcctggaacctgaagacacaggagattattattgtggagcctcgtactttactgtctgcgccaagagcatgcggaaaattgaatataggtactggggccaggggacccaggtcaccgtctcctcagaacccaagacaccaaaaccacaa

SEQ ID NO: 139 - protein, 284 aa, synthetic construct
  REGION 1..284: note = Description of Artificial Sequence: Synthetic polypeptide
QVQLVETGGGLVQAGGSLRLSCAASGRSYAMGWFRQGPGKEREFVATISWSSTNTWYADSVKGRFTISRDNAKNTVYLQMNSLKPEDTAVYYCAASHRFSDYPMRSEDGMDYWGKGTLVTVSSEPKTPKPQAIAGGGGSGGGGSGGGGSLQGQVQLVETGGGLVQAGGSLRLSCAASGRTFSSYSMGWFRQAPGKEREYVAAVNSNGDSTFYADSIKGRFTVSRDAAKNTVYLQMNSLKPEDTALYYCAAVYGRYTYQSPKSYEYWGQGTQVTVSSEPKTPKPQ

SEQ ID NO: 140 - DNA, 852 bp, synthetic construct
  misc_feature 1..852: note = Description of Artificial Sequence: Synthetic polynucleotide
  source 1..852: mol_type = other DNA
caggtgcagctggtggagacggggggaggattggtgcaggctgggggctctctgagactctcctgtgcagcctctggacgcagttatgccatgggctggttccgccagggtccagggaaggagcgtgagtttgtagccactatcagttggagtagtactaacacatggtatgcagattccgtgaagggccgattcaccatctctagagacaacgccaagaacacggtgtatctgcaaatgaacagcctgaaacctgaggacacggctgtttattactgtgcagcgagccatcgttttagcgactatcccatgaggtcagaggacggcatggactactggggcaaagggaccctggtcaccgtctcctcagaacccaagacaccaaaaccacaagcgatcgctggtggaggcggttcaggcggaggtggctctggcggtggcggttccctgcagggtcaggtgcagctggtggagacgggaggaggattggtgcaggctgggggctctctgagactctcgtgtgcagcctctggacgcaccttcagtagctattccatgggctggttccgccaggctccagggaaggagcgtgagtatgtagcagcagttaactccaatggcgacagtacattctatgccgactccattaagggccgattcaccgtctccagagacgccgccaagaacacagtctatctgcaaatgaacagcctgaaacctgaggacacggccctttattactgtgcagctgtctacggtagatacacttaccagtccccaaaatcgtatgagtactggggccaggggacccaggtcaccgtctcctcagaacccaagacaccaaaaccacaa

SEQ ID NO: 141 - protein, 289 aa, synthetic construct
  REGION 1..289: note = Description of Artificial Sequence: Synthetic polypeptide
QVQLVETGGGLVQAGGSLRLSCAASGRSYAMGWFRQGPGKEREFVATISWSSTNTWYADSVKGRFTISRDNAKNTVYLQMNSLKPEDTAVYYCAASHRFSDYPMRSEDGMDYWGKGTLVTVSSEPKTPKPQAIAGGGGSGGGGSGGGGSLQGQVQLVETGGGLVKPGGSLRLSCVVSGFTFDDYRMAWVRQAPGKELEWVSSIDSWSINTYYEDSVKGRFTISTDNAKNTLYLQMSSLKPEDTAVYYCAAEDRLGVPTINAHPSKYDYNYWGQGTQVTVSSEPKTPKPQ

SEQ ID NO: 142 - DNA, 867 bp, synthetic construct
  misc_feature 1..867: note = Description of Artificial Sequence: Synthetic polynucleotide
  source 1..867: mol_type = other DNA
caggtgcagctggtggagacggggggaggattggtgcaggctgggggctctctgagactctcctgtgcagcctctggacgcagttatgccatgggctggttccgccagggtccagggaaggagcgtgagtttgtagccactatcagttggagtagtactaacacatggtatgcagattccgtgaagggccgattcaccatctctagagacaacgccaagaacacggtgtatctgcaaatgaacagcctgaaacctgaggacacggctgtttattactgtgcagcgagccatcgttttagcgactatcccatgaggtcagaggacggcatggactactggggcaaagggaccctggtcaccgtctcctcagaacccaagacaccaaaaccacaagcgatcgctggtggaggcggttcaggcggaggtggctctggcggtggcggttccctgcagggtcaggtgcagctggtggagactggtggaggcttggtgaagcctgggggttctctgagactctcctgtgtagtctccggattcacttttgatgattatcgcatggcttgggtccgccaggctccagggaaggagctggagtgggtgtccagtatagatagttggagtatcaacacatactatgaagactccgtgaagggccggttcaccatctccacagacaacgccaagaatacactgtatctgcaaatgagcagcctgaaacctgaggacacggccgtgtattactgtgcagcagaggaccgcttaggtgtaccgactattaacgcccacccttcaaaatatgattataactactgggggcaggggacccaggtcaccgtctcctcagaacccaagacaccaaaaccacaa

SEQ ID NO: 143 - protein, 290 aa, synthetic construct
  REGION 1..290: note = Description of Artificial Sequence: Synthetic polypeptide
QLQLVETGGGLVKPGGSLRLSCVVSGFTFDDYRMAWVRQAPGKELEWVSSIDSWSINTYYEDSVKGRFTISTDNAKNTLYLQMSSLKPEDTAVYYCAAEDRLGVPTINAHPSKYDYNYWGQGTQVTVSSEPKTPKPQAIAGGGGSGGGGSGGGGSLQGQVQLVETGGGLVQAGGSLRLSCAASGRTFSSYSMGWFRQAPGKEREYVAAVNSNGDSTFYADSIKGRFTVSRDAAKNTVYLQMNSLKPEDTALYYCAAVYGRYTYQSPKSYEYWGQGTQVTVSSEPKTPKPQ

SEQ ID NO: 144 - DNA, 870 bp, synthetic construct
  misc_feature 1..870: note = Description of Artificial Sequence: Synthetic polynucleotide
  source 1..870: mol_type = other DNA
cagttgcagctcgtggagactggtggaggcttggtgaagcctgggggttctctgagactctcctgtgtagtctccggattcacttttgatgattatcgcatggcttgggtccgccaggctccagggaaggagctggagtgggtgtccagtatagatagttggagtatcaacacatactatgaagactccgtgaagggccggttcaccatctccacagacaacgccaagaatacactgtatctgcaaatgagcagcctgaaacctgaggacacggccgtgtattactgtgcagcagaggaccgcttaggtgtaccgactattaacgcccacccttcaaaatatgattataactactgggggcaggggacccaggtcaccgtctcctcagaacccaagacaccaaaaccacaagcgatcgctggtggaggcggttcaggcggaggtggctctggcggtggcggttccctgcagggtcaggtgcagctggtggagacgggaggaggattggtgcaggctgggggctctctgagactctcgtgtgcagcctctggacgcaccttcagtagctattccatgggctggttccgccaggctccagggaaggagcgtgagtatgtagcagcagttaactccaatggcgacagtacattctatgccgactccattaagggccgattcaccgtctccagagacgccgccaagaacacagtctatctgcaaatgaacagcctgaaacctgaggacacggccctttattactgtgcagctgtctacggtagatacacttaccagtccccaaaatcgtatgagtactggggccaggggacccaggtcaccgtctcctcagaacccaagacaccaaaaccacaa

SEQ ID NO: 145 - protein, 15 aa, synthetic construct
  REGION 1..15: note = Description of Artificial Sequence: Synthetic peptide
GGGGSGGGGSGGGGS

SEQ ID NO: 146 - protein, 13 aa, synthetic construct
  REGION 1..13: note = Description of Artificial Sequence: Synthetic peptide
DICLPRWGCLWED

SEQ ID NO: 147 - protein, 5 aa, synthetic construct
  REGION 1..5: note = Description of Artificial Sequence: Synthetic peptide
GGGGS

SEQ ID NO: 148 - protein, 111 aa, synthetic construct
  REGION 1..111: note = Description of Artificial Sequence: Synthetic polypeptide
SGGGLVQPGGSLRLSCAASGSSFSRYAMRWYRQAPGKQRELVANINSRGTSNYADSVKGRFTISRDNAKNTVYLQMNSLKPEDTAVYYCNAEWLGRSEPSWGQGTQVTVSS

SEQ ID NO: 149 - protein, 112 aa, synthetic construct
  REGION 1..112: note = Description of Artificial Sequence: Synthetic polypeptide
SGGGLVQPGGSLRLSCAASGFIFSLYTMRWHRQAPGKERELVATITSATGITNYADSVKGRFIISRDDAKKTGYLQMNSLKPEDTAVYYCNAVRTTVSRDYWGQGTQVTVSS

SEQ ID NO: 150 - protein, 111 aa, synthetic construct
  REGION 1..111: note = Description of Artificial Sequence: Synthetic polypeptide
SGGGLVQPGGSLRLSCAASGIIFSIYTMGWYRQAPGKQRELVAAIPSGPSANATDSVGGRFTITRDNAENTVYLQMNDLKPEDTAVYYCNARRGPGIKNYWGQGTQVTVSS

SEQ ID NO: 151 - protein, 116 aa, synthetic construct
  REGION 1..116: note = Description of Artificial Sequence: Synthetic polypeptide
SGGGLVQPGGSLSVSCAASGSIARPGAMAWYRQAPGKERELVASITPGGLTNYADSVTGRFTISRDNAKRTVYLQMNSLQPEDTAVYYCHARIIPLGLGSEYRDHWGQGTQVTVSS

SEQ ID NO: 152 - protein, 119 aa, synthetic construct
  REGION 1..119: note = Description of Artificial Sequence: Synthetic polypeptide
TGGLVQPGGSLRLSCAASGLTFSSTAMAWFRQAPGKEREFVARISGAGITIYYSDSVKDRFTISRNNVENTVYLQMNSLKTEDTAVYYCAARRNTYTSDYNIPARYPYWGQGTQVTVSS

SEQ ID NO: 153 - protein, 112 aa, synthetic construct
  REGION 1..112: note = Description of Artificial Sequence: Synthetic polypeptide
TGGLVQPGGSLRLSCAASRSTTATIYSMNWYRQAPGKQRELVAGMTSDGQTNYATSVKGRFTISRDNAKNTVYLIMNSLKLEDTAVYYCYVKPWRLQGWDYWGQGTQVTVSS

SEQ ID NO: 154 - protein, 108 aa, synthetic construct
  REGION 1..108: note = Description of Artificial Sequence: Synthetic polypeptide
SGGGLVQPGGSLRLSCAAPESIVNSRTMAWYRQAPGKQRERVATITTAGSPNYADSVKGRFAISRDNAKNTVYLQMNSLKPEDTAVYYCNTLLSTLPYGQGTQVTVSS

SEQ ID NO: 155 - protein, 109 aa, synthetic construct
  REGION 1..109: note = Description of Artificial Sequence: Synthetic polypeptide
SGGGLVQPGGSLGLSCVVASERSINNYGMGWYRQAPGKQRELVAQISSGGTTNYADSVEGRFTISRDNVKKMVHLQVNSLKPEDTAVYYCNSLLRTFSWGQGTQVTVSS

SEQ ID NO: 156 - protein, 112 aa, synthetic construct
  REGION 1..112: note = Description of Artificial Sequence: Synthetic polypeptide
TGGGLVQPGGSLRLSCAASGFTFSSYRMSWYRQAAGKERDVVATITANGVPTGYADSVMGRFTISRDNAKNTVYLEMNSLNPEDTAVYYCNAPRLHTSVGYWGQGTQVTVSS

SEQ ID NO: 157 - protein, 121 aa, synthetic construct
  REGION 1..121: note = Description of Artificial Sequence: Synthetic polypeptide
SGGGLVQAGNSLRLSCTASGVIFSIYTMGWFRQAPGKEREFVAAIGVADGTALVADSVTGRFTISRDNAKNTVYLHMNSLKPEDTAVYSCAAYLSPRVQSPYITDSRYQLWGQGTQVTVSS

SEQ ID NO: 158 - protein, 111 aa, synthetic construct
  REGION 1..111: note = Description of Artificial Sequence: Synthetic polypeptide
TGGGLVQAGGSLRLSCAASGRYAMGWFRQAPGKEREFVATISRSGAIREYADSVKGRFTISRDGAENTVYLEMNSLKPDDTAIYVCAEGRGATFNPEYAYWGQGTQVTVSS

SEQ ID NO: 159 - protein, 115 aa, synthetic construct
  REGION 1..115: note = Description of Artificial Sequence: Synthetic polypeptide
TGGGLVQAGGSLRLSCSASGLTFGNYAMGWFRQAPGKEREFVASISRSGSNTWYAEPLKGRFAISRDNDKNALYLQMNSLKPEDTAVYYCAGGSYNSDWWNYMYWGQGTQVTVSS

SEQ ID NO: 160 - protein, 128 aa, synthetic construct
  REGION 1..128: note = Description of Artificial Sequence: Synthetic polypeptide
SGGGGLVQAGGSLRLSCAASGRTFSGYAMGWFRQAPGKEREFVADISWSGHNTYYGDSVKGRFTISRDTAKNTVYLQMNSLKPEDTAVYYCAAEGARTHLSDSYYFPGLWAEPPVGYWGQGTQVTVSS

SEQ ID NO: 161 - protein, 117 aa, synthetic construct
  REGION 1..117: note = Description of Artificial Sequence: Synthetic polypeptide
TGGGLVQAGGTLRLSCAASGRTFTSYYIGWFRQEPGKEREFVASIGWTDDNTYYADSVKGRFTISRDNAETTAYLQMSGLKPEDTAVYYCAADYGSGIRAWYNWIYWGQGTQVTVSS

SEQ ID NO: 162 - protein, 118 aa, synthetic construct
  REGION 1..118: note = Description of Artificial Sequence: Synthetic polypeptide
TGGGLVQPGGSLRLSCAASGATLDTYIITWFRQAPGKEREAVSCINRSGSTTYSDSVKGRFTISRDNAQKTVYLQMNSLNPEDTAIYYCAADASYRTCGGSWWNWAYWGQGTQVTVSS

SEQ ID NO: 163 - protein, 116 aa, synthetic construct
  REGION 1..116: note = Description of Artificial Sequence: Synthetic polypeptide
SGGGSVQPGGSLRLSCAASGFTFSSYTMSWVRQAPGKGIEWVSDINGGGDRTDYADSVKGRFTISRDNARNTLYLQMNSLQPEDTAVYYCAKDLSYVSGTYFANDWGQGTQVTVSS

SEQ ID NO: 164 - protein, 112 aa, synthetic construct
  REGION 1..112: note = Description of Artificial Sequence: Synthetic polypeptide
SGGGLVQPGGSLRLSCTASGIIFDYYSVDWYRQAPGKERELVATITGDGSPNYADSVKGRFTISRDNAKKTVYLQMNGLKPEETAVYYCHAKRTIGTKSEYWGQGTQVTVSS

SEQ ID NO: 165 - protein, 109 aa, synthetic construct
  REGION 1..109: note = Description of Artificial Sequence: Synthetic polypeptide
TGGGLVQAGGSLRLSCLASRMSFSRRPMAWYRQAPGKQRERVATISSFGDTTNYTDSVEGRFTISRDNAKNTMYLQMNSLKPDDTAVYYCNTLLATYAWGQGTQVTVSS

SEQ ID NO: 166 - protein, 121 aa, synthetic construct
  REGION 1..121: note = Description of Artificial Sequence: Synthetic polypeptide
SGGGLVQAGGSLRLSCAASGRTFSSYVMGWFRQAPGKEREFVAAISRNGGKTYYADSVKGRFTISRDGTENTVYLQMNSLKPEDTAVYYCAAAVAASAEFVTARSNFYEYWGQGTQVTVSS

SEQ ID NO: 167 - protein, 490 aa, synthetic construct
  REGION 1..490: note = Description of Artificial Sequence: Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMAISDPNSGAPVPYPDPLEPRAAAQVQLAESGGGLVQPGGSLGLSCVVASERSINNYGMGWYRQAPGKQRELVAQISSGGTTNYADSVEGRFTISRDNVKKMVHLQVNSLKPEDTAVYYCNSLLRTFSWGQGTQVTVSSEPKTPKPQAIAGGGGSGGGGSGGGGSLQGQVQLVESGGGLVQPGGSLSVSCAASGSIARPGAMAWYRQAPGKERELVASITPGGLTNYADSVTGRFTISRDNAKRTVYLQMNSLQPEDTAVYYCHARIIPLGLGSEYRDHWGQGTQVTVSSAHHSEDPSARQGAPVPYPDPLEPRGGGSDICLPRWGCLWED

SEQ ID NO: 168 - protein, 503 aa, synthetic construct
  REGION 1..503: note = Description of Artificial Sequence: Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMAISDPNSGAPVPYPDPLEPRAAAQVQLAESGGGLVQPGGSLGLSCVVASERSINNYGMGWYRQAPGKQRELVAQISSGGTTNYADSVEGRFTISRDNVKKMVHLQVNSLKPEDTAVYYCNSLLRTFSWGQGTQVTVSSEPKTPKPQAIAGGGGSGGGGSGGGGSLQGQVQLAESGGGGLVQAGGSLRLSCAASGRTFSGYAMGWFRQAPGKEREFVADISWSGHNTYYGDSVKGRFTISRDTAKNTVYLQMNSLKPEDTAVYYCAAEGARTHLSDSYYFPGLWAEPPVGYWGQGTQVTVSSEPKTPKPQPARQGAPVPYPDPLEPRGGGSDICLPRWGCLWED

SEQ ID NO: 169 - protein, 6 aa, synthetic construct
  REGION 1..6: note = Description of Artificial Sequence: Synthetic 6xHis tag
HHHHHH

SEQ ID NO: 170 - protein, 9 aa, synthetic construct
  REGION 1..9: note = Description of Artificial Sequence: Synthetic peptide
SGSIARPGA

SEQ ID NO: 171 - protein, 9 aa, synthetic construct
  REGION 1..9: note = Description of Artificial Sequence: Synthetic peptide
SITPGGLTN

SEQ ID NO: 172 - protein, 16 aa, synthetic construct
  REGION 1..16: note = Description of Artificial Sequence: Synthetic peptide
HARIIPLGLGSEYRDH

SEQ ID NO: 173 - protein, 10 aa, synthetic construct
  REGION 1..10: note = Description of Artificial Sequence: Synthetic peptide
ASERSINNYG

SEQ ID NO: 174 - protein, 9 aa, synthetic construct
  REGION 1..9: note = Description of Artificial Sequence: Synthetic peptide
QISSGGTTN

SEQ ID NO: 175 - protein, 8 aa, synthetic construct
  REGION 1..8: note = Description of Artificial Sequence: Synthetic peptide
NSLLRTFS

SEQ ID NO: 176 - protein, 9 aa, synthetic construct
  REGION 1..9: note = Description of Artificial Sequence: Synthetic peptide
SGLTFGNYA

SEQ ID NO: 177 - protein, 10 aa, synthetic construct
  REGION 1..10: note = Description of Artificial Sequence: Synthetic peptide
SISRSGSNTW

SEQ ID NO: 178 - protein, 14 aa, synthetic construct
  REGION 1..14: note = Description of Artificial Sequence: Synthetic peptide
AGGSYNSDWWNYMY

SEQ ID NO: 179 - protein, 9 aa, synthetic construct
  REGION 1..9: note = Description of Artificial Sequence: Synthetic peptide
SGRTFSGYA

SEQ ID NO: 180 - protein, 10 aa, synthetic construct
  REGION 1..10: note = Description of Artificial Sequence: Synthetic peptide
DISWSGHNTY

SEQ ID NO: 181 - protein, 25 aa, synthetic construct
  REGION 1..25: note = Description of Artificial Sequence: Synthetic peptide
AEGARTHLSDSYYFPGLWAEPPVGY

SEQ ID NO: 182 - protein, 9 aa, synthetic construct
  REGION 1..9: note = Description of Artificial Sequence: Synthetic peptide
SGRTFTSYY

SEQ ID NO: 183 - protein, 10 aa, synthetic construct
  REGION 1..10: note = Description of Artificial Sequence: Synthetic peptide
SIGWTDDNTY

SEQ ID NO: 184 - protein, 16 aa, synthetic construct
  REGION 1..16: note = Description of Artificial Sequence: Synthetic peptide
AADYGSGIRAWYNWIY

SEQ ID NO: 185 - protein, 10 aa, synthetic construct
  REGION 1..10: note = Description of Artificial Sequence: Synthetic peptide
SGATLDTYII

SEQ ID NO: 186 - protein, 9 aa, synthetic construct
  REGION 1..9: note = Description of Artificial Sequence: Synthetic peptide
CINRSGSTT

SEQ ID NO: 187 - protein, 18 aa, synthetic construct
  REGION 1..18: note = Description of Artificial Sequence: Synthetic peptide
AADASYRTCGGSWWNWAY

SEQ ID NO: 188 - protein, 9 aa, synthetic construct
  REGION 1..9: note = Description of Artificial Sequence: Synthetic peptide
SGFTFSSYT

SEQ ID NO: 189 - protein, 10 aa, synthetic construct
  REGION 1..10: note = Description of Artificial Sequence: Synthetic peptide
DINGGGDRTD

SEQ ID NO: 190 - protein, 15 aa, synthetic construct
  REGION 1..15: note = Description of Artificial Sequence: Synthetic peptide
AKDLSYVSGTYFAND

SEQ ID NO: 191 - protein, 10 aa, synthetic construct
  REGION 1..10: note = Description of Artificial Sequence: Synthetic peptide
SGIIFDYYSV

SEQ ID NO: 192 - protein, 9 aa, synthetic construct
  REGION 1..9: note = Description of Artificial Sequence: Synthetic peptide
TITGDGSPN

SEQ ID NO: 193 - protein, 12 aa, synthetic construct
  REGION 1..12: note = Description of Artificial Sequence: Synthetic peptide
HAKRTIGTKSEY

SEQ ID NO: 194 - protein, 9 aa, synthetic construct
  REGION 1..9: note = Description of Artificial Sequence: Synthetic peptide
SRMSFSRRP

SEQ ID NO: 195 - protein, 10 aa, synthetic construct
  REGION 1..10: note = Description of Artificial Sequence: Synthetic peptide
TISSFGDTTN

SEQ ID NO: 196 - protein, 8 aa, synthetic construct
  REGION 1..8: note = Description of Artificial Sequence: Synthetic peptide
NTLLATYA

SEQ ID NO: 197 - protein, 9 aa, synthetic construct
  REGION 1..9: note = Description of Artificial Sequence: Synthetic peptide
SGRTFSSYV

SEQ ID NO: 198 - protein, 10 aa, synthetic construct
  REGION 1..10: note = Description of Artificial Sequence: Synthetic peptide
AISRNGGKTY

SEQ ID NO: 199 - protein, 20 aa, synthetic construct
  REGION 1..20: note = Description of Artificial Sequence: Synthetic peptide
AAAVAASAEFVTARSNFYEY

SEQ ID NO: 200 - protein, 18 aa, synthetic construct
  REGION 1..18: note = Description of Artificial Sequence: Synthetic peptide
  VARIANT 1: note = T or S
  VARIANT 5: note = G or absent
  VARIANT 6: note = L or S
  VARIANT 9: note = A or P
  VARIANT 14: note = R, S or G
  VARIANT 15: note = L or V
  VARIANT 18: note = A, V, S, T or L
XGGGXXVQXGGSLXXSCX

SEQ ID NO: 201 - protein, 14 aa, synthetic construct
  REGION 1..14: note = Description of Artificial Sequence: Synthetic peptide
  VARIANT 1: note = G, A, T, S or D
  VARIANT 3: note = F, Y or V
  VARIANT 6: note = A or E
  VARIANT 10: note = E, Q or G
  VARIANT 11: note = R or I
  VARIANT 13: note = F, L, A, W or R
XWXRQXPGKXXEXV

SEQ ID NO: 202 - protein, 36 aa, synthetic construct
  REGION 1..36: note = Description of Artificial Sequence: Synthetic polypeptide
  VARIANT 2: note = G, A, S or T
  VARIANT 3: note = D or E
  VARIANT 4: note = S or P
  VARIANT 5: note = V or L
  VARIANT 6: note = K, T or E
  VARIANT 10: note = T or A
  VARIANT 15: note = T, N or G
  VARIANT 16: note = A, V, D or T
  VARIANT 17: note = K, E, Q or R
  VARIANT 18: note = N, R, K or T
  VARIANT 19: note = T, A or M
  VARIANT 20: note = V, L, A or M
  VARIANT 21: note = Y or H
  VARIANT 24: note = M or V
  VARIANT 25: note = N or S
  VARIANT 26: note = S or G
  VARIANT 28: note = K, Q or N
  VARIANT 30: note = E or D
  VARIANT 31: note = D or E
  VARIANT 34: note = V or I
YXXXXXGRFXISRDXXXXXXXLQXXXLXPXXTAXYY

SEQ ID NO: 203 - protein, 10 aa, synthetic construct
  REGION 1..10: note = Description of Artificial Sequence: Synthetic peptide
GQGTQVTVSS

SEQ ID NO: 204 - protein, 18 aa, synthetic construct
  REGION 1..18: note = Description of Artificial Sequence: Synthetic peptide
SGGGGLVQAGGSLRLSCA

SEQ ID NO: 205 - protein, 14 aa, synthetic construct
  REGION 1..14: note = Description of Artificial Sequence: Synthetic peptide
GWFRQAPGKEREFV

SEQ ID NO: 206 - protein, 36 aa, synthetic construct
  REGION 1..36: note = Description of Artificial Sequence: Synthetic polypeptide
YGDSVKGRFTISRDTAKNTVYLQMNSLKPEDTAVYY

SEQ ID NO: 207 - protein, 17 aa, synthetic construct
  REGION 1..17: note = Description of Artificial Sequence: Synthetic peptide
SGGGLVQPGGSLSVSCA

SEQ ID NO: 208 - protein, 17 aa, synthetic construct
  REGION 1..17: note = Description of Artificial Sequence: Synthetic peptide
SGGGLVQPGGSLGLSCV

SEQ ID NO: 209 - protein, 17 aa, synthetic construct
  REGION 1..17: note = Description of Artificial Sequence: Synthetic peptide
TGGGLVQAGGSLRLSCS

SEQ ID NO: 210 - protein, 17 aa, synthetic construct
  REGION 1..17: note = Description of Artificial Sequence: Synthetic peptide
TGGGLVQAGGTLRLSCA

SEQ ID NO: 211 - protein, 17 aa, synthetic construct
  REGION 1..17: note = Description of Artificial Sequence: Synthetic peptide
TGGGLVQPGGSLRLSCA

SEQ ID NO: 212 - protein, 17 aa, synthetic construct
  REGION 1..17: note = Description of Artificial Sequence: Synthetic peptide
SGGGSVQPGGSLRLSCA

SEQ ID NO: 213 - protein, 17 aa, synthetic construct
  REGION 1..17: note = Description of Artificial Sequence: Synthetic peptide
SGGGLVQPGGSLRLSCT

SEQ ID NO: 214 - protein, 17 aa, synthetic construct
  REGION 1..17: note = Description of Artificial Sequence: Synthetic peptide
TGGGLVQAGGSLRLSCL

SEQ ID NO: 215 - protein, 17 aa, synthetic construct
  REGION 1..17: note = Description of Artificial Sequence: Synthetic peptide
SGGGLVQAGGSLRLSCA

SEQ ID NO: 216 - protein, 14 aa, synthetic construct
  REGION 1..14: note = Description of Artificial Sequence: Synthetic peptide
AWYRQAPGKERELV

SEQ ID NO: 217 - protein, 14 aa, synthetic construct
  REGION 1..14: note = Description of Artificial Sequence: Synthetic peptide
GWYRQAPGKQRELV

SEQ ID NO: 218 - protein, 14 aa, synthetic construct
  REGION 1..14: note = Description of Artificial Sequence: Synthetic peptide
GWFRQEPGKEREFV

SEQ ID NO: 219 - protein, 14 aa, synthetic construct
  REGION 1..14: note = Description of Artificial Sequence: Synthetic peptide
TWFRQAPGKEREAV

SEQ ID NO: 220 - protein, 14 aa, synthetic construct
  REGION 1..14: note = Description of Artificial Sequence: Synthetic peptide
SWVRQAPGKGIEWV

SEQ ID NO: 221 - protein, 14 aa, synthetic construct
  REGION 1..14: note = Description of Artificial Sequence: Synthetic peptide
DWYRQAPGKERELV

SEQ ID NO: 222 - protein, 14 aa, synthetic construct
  REGION 1..14: note = Description of Artificial Sequence: Synthetic peptide
AWYRQAPGKQRERV

SEQ ID NO: 223 - protein, 36 aa, synthetic construct
  REGION 1..36: note = Description of Artificial Sequence: Synthetic polypeptide
YADSVTGRFTISRDNAKRTVYLQMNSLQPEDTAVYY

SEQ ID NO: 224 - protein, 36 aa, synthetic construct
  REGION 1..36: note = Description of Artificial Sequence: Synthetic polypeptide
YADSVEGRFTISRDNVKKMVHLQVNSLKPEDTAVYY

SEQ ID NO: 225 - protein, 36 aa, synthetic construct
  REGION 1..36: note = Description of Artificial Sequence: Synthetic polypeptide
YAEPLKGRFAISRDNDKNALYLQMNSLKPEDTAVYY

SEQ ID NO: 226 - protein, 36 aa, synthetic construct
  REGION 1..36: note = Description of Artificial Sequence: Synthetic polypeptide
YADSVKGRFTISRDNAETTAYLQNSGLKPEDTAVYY

SEQ ID NO: 227 - protein, 36 aa, synthetic construct
  REGION 1..36: note = Description of Artificial Sequence: Synthetic polypeptide
YSDSVKGRFTISRDNAQKTVYLQMNSLNPEDTAIYY

SEQ ID NO: 228 - protein, 36 aa, synthetic construct
  REGION 1..36: note = Description of Artificial Sequence: Synthetic polypeptide
YADSVKGRFTISRDNARNTLYLQMNSLQPEDTAVYY

SEQ ID NO: 229 - protein, 36 aa, synthetic construct
  REGION 1..36: note = Description of Artificial Sequence: Synthetic polypeptide
YADSVKGRFTISRDNAKKTVYLQMNGLKPEETAVYY

SEQ ID NO: 230 - protein, 36 aa, synthetic construct
  REGION 1..36: note = Description of Artificial Sequence: Synthetic polypeptide
YTDSVEGRFTISRDNAKNTMYLQMNSLKPDDTAVYY

SEQ ID NO: 231 - protein, 36 aa, synthetic construct
  REGION 1..36: note = Description of Artificial Sequence: Synthetic polypeptide
YADSVKGRFTISRDGTENTVYLQMNSLKPEDTAVYY

SEQ ID NO: 232 - protein, 9 aa, synthetic construct
  REGION 1..9: note = Description of Artificial Sequence: Synthetic peptide
SGSSFSRYA

SEQ ID NO: 233 - protein, 9 aa, synthetic construct
  REGION 1..9: note = Description of Artificial Sequence: Synthetic peptide
SGFIFSLYT

SEQ ID NO: 234 - protein, 9 aa, synthetic construct
  REGION 1..9: note = Description of Artificial Sequence: Synthetic peptide
SGIIFSIYT

SEQ ID NO: 235 - protein, 9 aa, synthetic construct
  REGION 1..9: note = Description of Artificial Sequence: Synthetic peptide
SGLTFSSTA

SEQ ID NO: 236 - protein, 10 aa, synthetic construct
  REGION 1..10: note = Description of Artificial Sequence: Synthetic peptide
SRSTTATIYS

SEQ ID NO: 237 - protein, 9 aa, synthetic construct
  REGION 1..9: note = Description of Artificial Sequence: Synthetic peptide
PESIVNSRT

SEQ ID NO: 238 - protein, 9 aa, synthetic construct
  REGION 1..9: note = Description of Artificial Sequence: Synthetic peptide
SGFTFSSYR

SEQ ID NO: 239 - protein, 9 aa, synthetic construct
  REGION 1..9: note = Description of Artificial Sequence: Synthetic peptide
SGVIFSIYT

SEQ ID NO: 240 - protein, 5 aa, synthetic construct
  REGION 1..5: note = Description of Artificial Sequence: Synthetic peptide
SGRYA

SEQ ID NO: 241 - protein, 9 aa, synthetic construct
  REGION 1..9: note = Description of Artificial Sequence: Synthetic peptide
NINSRGTSN

SEQ ID NO: 242 - protein, 10 aa, synthetic construct
  REGION 1..10: note = Description of Artificial Sequence: Synthetic peptide
TITSATGITN

SEQ ID NO: 243 - protein, 9 aa, synthetic construct
  REGION 1..9: note = Description of Artificial Sequence: Synthetic peptide
AIPSGPSAN

SEQ ID NO: 244 - protein, 10 aa, synthetic construct
  REGION 1..10: note = Description of Artificial Sequence: Synthetic peptide
RISGAGITIY

SEQ ID NO: 245 - protein, 9 aa, synthetic construct
  REGION 1..9: note = Description of Artificial Sequence: Synthetic peptide
GMTSDGQTN

SEQ ID NO: 246 - protein, 9 aa, synthetic construct
  REGION 1..9: note = Description of Artificial Sequence: Synthetic peptide
TITTAGSPN

SEQ ID NO: 247 - protein, 10 aa, synthetic construct
  REGION 1..10: note = Description of Artificial Sequence: Synthetic peptide
TITANGVPTG

SEQ ID NO: 248 - protein, 10 aa, synthetic construct
  REGION 1..10: note = Description of Artificial Sequence: Synthetic peptide
AIGVADGTAL

SEQ ID NO: 249 - protein, 10 aa, synthetic construct
  REGION 1..10: note = Description of Artificial Sequence: Synthetic peptide
TISRSGAIRE

SEQ ID NO: 250 - protein, 11 aa, synthetic construct
  REGION 1..11: note = Description of Artificial Sequence: Synthetic peptide
NAEWLGRSEPS

SEQ ID NO: 251 - protein, 11 aa, synthetic construct
  REGION 1..11: note = Description of Artificial Sequence: Synthetic peptide
NAVRTTVSRDY

SEQ ID NO: 252 - protein, 11 aa, synthetic construct
  REGION 1..11: note = Description of Artificial Sequence: Synthetic peptide
NARRGPGIKNY

SEQ ID NO: 253 - protein, 19 aa, synthetic construct
  REGION 1..19: note = Description of Artificial Sequence: Synthetic peptide
AARRNTYTSDYNIPARYPY

SEQ ID NO: 254 - protein, 12 aa, synthetic construct
  REGION 1..12: note = Description of Artificial Sequence: Synthetic peptide
YVKPWRLQGWDY

SEQ ID NO: 255 - protein, 8 aa, synthetic construct
  REGION 1..8: note = Description of Artificial Sequence: Synthetic peptide
NTLLSTLP

SEQ ID NO: 256 - protein, 11 aa, synthetic construct
  REGION 1..11: note = Description of Artificial Sequence: Synthetic peptide
NAPRLHTSVGY

SEQ ID NO: 257 - protein, 20 aa, synthetic construct
  REGION 1..20: note = Description of Artificial Sequence: Synthetic peptide
AAYLSPRVQSPYITDSRYQL

SEQ ID NO: 258 - protein, 13 aa, synthetic construct
  REGION 1..13: note = Description of Artificial Sequence: Synthetic peptide
EGRGATFNPEYAY

SEQ ID NO: 259 - protein, 26 aa, synthetic construct
  REGION 1..26: note = Description of Artificial Sequence: Synthetic peptide
AAEGARTHLSDSYYFPGLWAEPPVGY

(19) United States
(12) Patent Application Publication
(10) Pub. No.: US 2025/0042978 A1
(43) Pub. Date: Feb. 6, 2025

(54) VHH BASED BINDING ANTIBODIES FOR ANTHRAX AND BOTULINUM TOXINS AND METHODS OF MAKING AND USING THEREFOR

(71) Applicant: TRUSTEES OF TUFTS COLLEGE, MEDFORD, MA (US)

(72) Inventor: CHARLES B. SHOEMAKER, MEDFORD, MA (US)

(73) Assignee: TRUSTEES OF TUFTS COLLEGE, MEDFORD, MA (US)

(21) Appl. No.: 18/768,704

(22) Filed: Jul. 10, 2024

Related U.S. Application Data
(63) Continuation of application No. 17/221,177, filed on Apr. 2, 2021, now Pat. No. 12,065,486.
Application No. 17/221,177 is a division of application No. 15/534,776, filed on Jun. 9, 2017, now Pat. No. 11,001,625.
(60) Provisional application No. 62/089,949, filed on Dec. 10, 2014.

(51) Int. Cl. C07K 16/12; A61K 39/00

(52) U.S. Cl. CPC C07K 16/1282; C07K 16/1278; C12Y 304/24069; C12Y 304/24083; A61K 2039/505; C07K 2317/31; C07K 2317/569; C07K 2317/76; G01N 2333/952; G01N 2333/954

(57) ABSTRACT

Methods, compositions and kits are provided for treating a subject exposed to or at risk for exposure to a disease agent, methods, compositions and kits having a pharmaceutical composition including at least one recombinant binding protein or a source of expression of the binding protein, wherein the binding protein neutralizes at least one or a plurality of disease agents that are toxins, for example at least one of a Botulinum toxin or an Anthrax toxin.

### CROSS REFERENCE TO RELATED APPLICATIONS

This application is a continuation of U.S. application Ser. No. 17/221,177, filed on Apr. 2, 2021, which is a divisional of U.S. application Ser. No. 15/534,776, filed on Jun. 9, 2017, and issued as U.S. Pat. No. 11,001,625 on May 11, 2021, which is the U.S. national stage application pursuant to 35 U.S.C. § 371 of International PCT Application No. PCT/US2015/064872, filed on Dec. 10, 2015, designating the United States and published in English, which claims priority to and benefit of U.S. Provisional Application No. 62/089,949, filed on Dec. 10, 2014, the contents of all of which are incorporated by reference herein in their entireties.

### GOVERNMENT SUPPORT

This invention was made with government support under grant numbers AI057159 and AI093467 awarded by the National Institutes of Health. The government has certain rights in the invention.

### SEQUENCE LISTING

This application contains a Sequence Listing which has been submitted electronically in XML format and is hereby incorporated by reference in its entirety. The Sequence Listing XML file, created on Jul. 8, 2024, is named 167774_011404_US_SL.xml and is 392,218 bytes in size.

### TECHNICAL FIELD

The present invention generally relates to compositions and methods to prevent or treat exposure to Anthrax toxin or to Botulinum toxins by VHH based neutralizing antibodies.

### BACKGROUND

The disease, Anthrax is caused by the gram-positive bacterium Bacillus anthracis and is a major bioterror concern. Following introduction into a host in spore form and germination of the spore, the bacterium divides and manifests disease and lethality primarily through the action of two toxins, anthrax lethal toxin (LT) and edema toxin (ET). Anthrax toxins have a common receptor-binding component, protective antigen (PA), which is responsible for transport of the lethal factor metalloprotease (LF) or edema factor adenylate cyclase (EF) or both into the host cell cytosol. Injection of the toxins into animals replicates symptoms of anthrax disease.

PA acts as a ‘gateway’ that allows translocation and action of both LT and ET toxins and hence PA has been the primary target of therapeutics including antibodies developed for treatment of anthrax. PA binds to two cellular receptors as an 83 kDa polypeptide (PA83) and is rapidly cleaved by cell surface proteases such as furin to a 63 kDa (PA63) form which associates as heptamers or octamers that provide the binding sites for LF or EF. The oligomer bound to one or more molecules of LF/EF is then rapidly translocated. PA63 form of the Anthrax toxin is competent for endocytosis. When PA is cleaved prior to exposure to cells, or produced as PA63, it rapidly oligomerizes and the pre-formed oligomer binds and transports LF/EF into cells. The PA63 oligomer undergoes a conformational change in acidic endosomes to a heat and SDS-stable form, which allows the translocation of LF and EF through a central pore into the cytosol. LF and EF then act on their substrates and manifest toxic effects.

During anthrax infection, the accumulation of anthrax toxins in the blood leads to lethality. Antibodies against PA are considered a primary therapeutic for treatment of the disease. The majority of neutralizing antibodies developed against PA act on the receptor-binding domain to inhibit interaction of the toxin with cells. A few antibodies have been identified which neutralize PA by other mechanisms.

Botulinum toxin is a neurotoxin produced by the bacterium Clostridium botulinum. Botulinum toxin is released by C. botulinum spores, which are commonly found in soil and water. The C. botulinum spores produce botulinum toxin on exposure to low oxygen levels and certain temperatures. Botulinum toxin can cause Botulism, which is a serious and life-threatening paralytic illness in humans and animals. The early symptoms of Botulism are weakness, trouble seeing, feeling tired, and trouble speaking followed by weakness of the arms, chest muscles and legs. Botulinum toxin is an acute lethal toxin with an estimated human median lethal dose (LD-50) of 1.3-2.1 ng/kg intravenously or intramuscularly and 10-13 ng/kg when inhaled. Antibodies against Botulinum toxins are considered a primary therapeutic for treatment of the disease.

A need exists for generating high affinity binding agents that treat both routine incidents of disease and toxicity. The production of antibodies and their storage is a costly and lengthy process. In fact, development of a single antibody therapeutic agent often requires years of clinical study. Yet multiple, different therapeutic antibodies are necessary for the effective treatment of patients exposed to a bio-terrorist assault with a potential weapon such as Anthrax or Botulism. Developing and producing multiple antibodies each of which can bind to a different target (e.g. microbial pathogens, viral pathogens, and toxins) is often a difficult task because it involves separately producing, storing and transporting each of the multiplicity of antibodies of which each is specific for one pathogen or toxin. Production and stockpiling a sufficient amount of antibodies to protect large populations is a challenge and has not currently been achieved. The shelf life of antibodies is often relatively short (e.g., weeks or months), and accordingly freshly prepared batches of present therapeutic antibodies have to be produced to replace expiring antibodies.

Accordingly, there is a need for a cost effective and efficient way to provide alternatives to current therapeutic agents. Further a need exists for alternative therapeutics that are easier to develop and produce, have a longer shelf life, and bind as a single agent to multiple targets on the same disease agent, as well as to different disease agents.

### SUMMARY

An aspect of invention provides a pharmaceutical composition for treating a subject at risk for exposure to or exposed to at least one disease agent, the pharmaceutical composition including: at least one recombinant binding protein that neutralizes the disease agent and treats the subject for exposure to the disease agent, the binding protein including at least one amino acid sequence selected from the group of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, and SEQ ID NO: 143.

In some embodiments, the composition comprises at least one nucleotide sequence that encodes a recombinant binding protein having an amino acid sequence selected from the group consisting of: SEQ ID NO: 2, SEQ ID NO: 4 SEQ ID NO: 6, SEQ ID NO: 8, SEQ ID NO: 10, SEQ ID NO: 12, SEQ ID NO: 14 SEQ ID NO: 16, SEQ ID NO: 18, SEQ ID NO: 20, SEQ ID NO: 22, SEQ ID NO: 24 SEQ ID NO: 26, SEQ ID NO: 28, SEQ ID NO: 30, SEQ ID NO: 32, SEQ ID NO: 34 SEQ ID NO: 36, SEQ ID NO: 38, SEQ ID NO: 40, SEQ ID NO: 42, SEQ ID NO: 44 SEQ ID NO: 46, SEQ ID NO: 48, SEQ ID NO: 50, SEQ ID NO: 52, SEQ ID NO: 54 SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, and SEQ ID NO: 144. In an embodiment of the composition, the binding protein is heteromultimeric and has a plurality of binding regions. In another embodiment of the composition, the binding regions are not identical, and each binding region has affinity to specifically bind and neutralize a non-overlapping portion of the disease agent.

In an embodiment of the composition, the binding protein further includes at least one of: a tag epitope that has affinity to bind an antibody; and a linker that separates the binding regions, and the linker including at least one selected from the group of: a peptide, a protein, a sugar, and a nucleic acid. In an embodiment of the composition, the disease agent is a toxin selected from a plant lectin and a bacterial toxin. In some embodiments, the bacterial toxin is at least one selected from a B. anthracis toxin, a C. botulinum B toxin, and a C. botulinum E toxin. In an embodiment of the composition, the bacterial toxin is a B. anthracis toxin and the binding protein binds to and neutralizes at least one selected from: an Anthrax protective antigen, an Anthrax lethal toxin, and an Anthrax edema toxin. In some embodiments, the binding protein inhibits or prevents endocytosis of the toxin. In some embodiments, the Anthrax protective antigen is a cell surface generated antigen.

In various embodiments of the composition, the binding protein comprises an amino acid sequence that is substantially identical to a binding protein having an amino acid sequence as set forth in a SEQ ID NO: listed above, and has at least 50% identity, at least 60% identity, at least 65% identity, at least 70% identity, at least 75% identity, at least 80% identity, at least 85% identity, at least 90% identity, or and at least 95% identity to the amino acid sequence as set forth in a SEQ ID NO: listed above. In some embodiments, the nucleotide sequence encodes a binding protein comprising an amino acid sequence that is substantially identical to a binding protein having an amino acid sequence as set forth in a SEQ ID NO: listed above, and has at least 50% identity, at least 60% identity, at least 65% identity, at least 70% identity, at least 75% identity, at least 80% identity, at least 85% identity, at least 90% identity, or and at least 95% identity to a nucleotide sequence encoding a binding protein having an amino acid sequence as set forth in a SEQ ID NO: as listed above.

An aspect of the invention provides a method for treating a subject at risk for exposure to or exposed to at least one disease agent, the method including: administering to the subject at least one binding protein having at least one binding region including an amino acid sequence selected from: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53,SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, and SEQ ID NO: 143, and measuring a decrease in at least one symptom associated with exposure to disease agent.

In an embodiment of the method, measuring the symptom further comprises analyzing an amount of remediation of at least one symptom selected from fever, chills, swelling of neck, soreness of neck glands, sore throat, painful swallowing, hoarseness, nausea, vomiting, bloody vomiting, diarrhea, bloody diarrhea, constipation, headache, flushing, red eyes, stomach pain, fainting, swelling of abdomen, double vision, blurred vision, drooping eyelids, slurred speech, dry mouth, and muscle weakness.

An aspect of the invention provides a method of identifying a therapeutic binding protein for treating a subject at risk for exposure to or exposed to at least one disease agent, the method including: contacting a first sample of a disease agent with a test protein and measuring an amount of binding of the disease agent to the test protein under conditions for the disease agent to interact with the test protein; and comparing the amount of binding to that of a second sample of the disease agent not contacted by the test protein and otherwise identical, such that presence of the therapeutic binding protein is identified by an increase of binding of the disease agent in the first sample compared to the second sample.

In an embodiment of the method, the test protein is a plurality of proteins. In some embodiments, the disease agent is in vitro. In other embodiments, the disease agent is in a cell.

An embodiment of the method further includes contacting the disease agent to a mammalian subject and measuring a decrease in at least one symptom of the disease agent. In some embodiments, the disease agent is a toxin selected from a plant lectin and a bacterial toxin. In some embodiments, the bacterial toxin is at least one selected from a B. anthracis toxin, a C. botulinum B toxin, and a C. botulinum E toxin. In some embodiments, the bacterial toxin is a B. anthracis toxin and the binding protein binds to and neutralizes Anthrax protective antigen. In an embodiment, the binding protein inhibits or prevents endocytosis of the toxin. In an embodiment of the method, the Anthrax protective antigen is a cell surface generated antigen.

An aspect of the invention provides a method for treating a subject at risk for exposure to or exposed to at least one disease agent, the method including: administering to the subject a source of expression of a binding protein having a nucleotide sequence encoding the binding protein, such that the nucleotide sequence comprises at least one selected from the group consisting of: a naked nucleic acid vector, bacterial vector, and a viral vector, such that the nucleotide sequence encodes a binding protein having an amino acid sequence selected from the group consisting of: SEQ ID NO: 2, SEQ ID NO: 4 SEQ ID NO: 6, SEQ ID NO: 8, SEQ ID NO: 10, SEQ ID NO: 12, SEQ ID NO: 14 SEQ ID NO: 16, SEQ ID NO: 18, SEQ ID NO: 20, SEQ ID NO: 22, SEQ ID NO: 24 SEQ ID NO: 26, SEQ ID NO: 28, SEQ ID NO: 30, SEQ ID NO: 32, SEQ ID NO: 34 SEQ ID NO: 36, SEQ ID NO: 38, SEQ ID NO: 40, SEQ ID NO: 42, SEQ ID NO: 44 SEQ ID NO: 46, SEQ ID NO: 48, SEQ ID NO: 50, SEQ ID NO: 52, SEQ ID NO: 54 SEQ ID NO: 56, SEQ ID NO: 58, SEQ ID NO: 60, SEQ ID NO: 62, SEQ ID NO: 64 SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, and SEQ ID NO: 144.

An aspect of the invention provides a kit for treating a subject exposed to or at risk for exposure to a disease agent including: a unit dosage of a pharmaceutical composition for treating a subject at risk for exposure to or exposed to at least one disease agent, the pharmaceutical composition including: at least one recombinant binding protein that neutralizes the disease agent thereby treating the subject for exposure to the disease agent, such that the binding protein includes at least one amino acid sequence selected from the group of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, and SEQ ID NO: 143.

In an embodiment of the kit, the recombinant binding protein is encoded by at least one nucleotide sequence selected from the group consisting of: SEQ ID NO: 2, SEQ ID NO: 4 SEQ ID NO: 6, SEQ ID NO: 8, SEQ ID NO: 10, SEQ ID NO: 12, SEQ ID NO: 14 SEQ ID NO: 16, SEQ ID NO: 18, SEQ ID NO: 20, SEQ ID NO: 22, SEQ ID NO: 24 SEQ ID NO: 26, SEQ ID NO: 28, SEQ ID NO: 30, SEQ ID NO: 32, SEQ ID NO: 34 SEQ ID NO: 36, SEQ ID NO: 38, SEQ ID NO: 40, SEQ ID NO: 42, SEQ ID NO: 44 SEQ ID NO: 46, SEQ ID NO: 48, SEQ ID NO: 50, SEQ ID NO: 52, SEQ ID NO: 54 SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, and SEQ ID NO: 144.

An aspect of the invention provides a method for detecting a presence of a toxin in a sample, the method including: contacting and incubating an aliquot of the sample to an amount of at least one binding protein that specifically binds the toxin, such that the binding protein includes a binding region having an amino acid sequence selected from: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, and SEQ ID NO: 143, such that the toxin is selected from the group of: a B. anthracis toxin, a C. botulinum B toxin and a C. botulinum E toxin, and SEQ ID NO: 1, SEQ ID NO: 3 SEQ ID NO: 5, SEQ ID NO: 7, SEQ ID NO: 9, SEQ ID NO: 11, SEQ ID NO: 13, SEQ ID NO: 15, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, and SEQ ID NO: 105 are amino acid sequences of binding proteins that specifically bind a B. anthracis toxin, and SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 131, SEQ ID NO: 133, SEQ ID NO: 135, and SEQ ID NO: 137 are amino acid sequences of binding proteins that specifically bind a B. botulinum B toxin, and SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 127, SEQ ID NO: 129, SEQ ID NO: 139, SEQ ID NO: 141, and SEQ ID NO: 143 are amino acid sequences of binding proteins that specifically bind a B. botulinum E toxin under conditions to form a complex; separating the complex from unbound binding protein; and measuring amount of complex formed.

In an embodiment of the method, the sample is at least one selected from: a medical sample, a food sample, a beverage sample, a water sample, and an environmental sample. In another embodiment of the invention, the medical sample is at least one selected from: blood, plasma, tissue, stool, urine, perspiration, serum, semen, breast milk, cerebrospinal fluid, skin and hair. An embodiment of the method further includes, analyzing the extent of complex formation, such that the extent of complex formation is a function of extent of toxin present in the sample.

### BRIEF DESCRIPTION OF THE DRAWINGS

FIG. 1A-FIG. 1C are Meyer-Kaplan survival plots showing percent survival (% survival, ordinate) of subjects as a function of time in days (abscissa) following contact with LT and VHH binding/neutralizing agents as indicated. Balb/cJ mice were injected intravenously with antibody (Ab) at indicated molar ratios (Ab: toxin) 10 min prior to injection with LT (45 μg for each toxin component, IV). FIG. 1C shows a single group (POST) received antibody 2 hours after toxin was administered. Control groups were injected with PBS instead of antibody. Heterodimers JKC-5 and VNA1 (JKD-11) contain the VHHs JIK-B8 and JIJ-B8, both with high affinity for PA but only JIK-B8 having anthrax neutralizing properties. VNA2 contains JIK-B8 and JKH-C7, both potent anthrax neutralizing VHHs. Heterodimers VNA1 and VNA2 also contain a carboxyl albumin-binding-peptide (ABP) which prolongs in vivo serum persistence of similar VNAs in mice. Antibody 14B7 is a previously described neutralizing monoclonal antibody, which binds to the same receptor-interacting domain as JIK-B8. Animals were monitored for 10 days post for signs of malaise and survival.

FIG. 2A and FIG. 2D are Meyer-Kaplan survival plots showing percent survival (% survival, ordinate) of subjects as a function of time in days (abscissa) following contact with A35 Sterne-like toxigenic B. anthracis strain spores (2×10⁷ spores, SC) and VHH binding/neutralizing agents. FIG. 2A shows data obtained using C57BL/6J mice treated with VNA2 heterodimeric VNA or 14B7 mAb control (15 μg/injection/IV) at indicated times before or after spore infection. FIG. 2B shows data obtained after antibody was administered post-infection at indicated times and doses. Control mice were treated with PBS at 15 min, 1 hour and 4 hours post infection. FIG. 2C contains data from Balb/cJ mice injected intravenously (IV) with antibody at indicated molar ratios (Ab: toxin) 10 minutes prior to injection with LT (45 μg for each toxin component). Control groups received PBS instead of antibody. Animals were monitored for 10 days post treatment for signs of malaise and survival. FIG. 2D shows data from C57BL/6J mice (n=5/group, except PBS controls, n=15), treated with heterodimeric VNA2-PA subcutaneously (SC) at indicated times and doses before or after spore infection (2×10⁷ spores, also SC at a distal site). Control mice were treated with PBS at 15 min, one hour and four hours post infection (n=5) or at five minutes (n=5) and eight hours (n=5) post infection. Neutralizing mAb 14B7 was used as a positive control in these studies. Mice were monitored for survival and signs of malaise for 10 days.

FIG. 3 shows amino acid sequences SEQ ID NOS 148-166 of VHHs selected for binding to anthrax PA. Sequences shown begin within framework 1 at the site of the primer binding employed in coding sequence DNA amplification from the immune alpaca cDNA and continue through the end of framework 4. The expression in parentheses at the right end indicates that the VHH contains a long hinge (lh) or a short hinge (sh). The locations of each of the three complementarity determining regions (CDRs) are indicated at the top end. FIG. 3 shows amino acid sequences SEQ ID NOS 148-166 of VHHs selected for binding to anthrax PA. Sequences shown begin within framework 1 at the site of the primer binding employed in coding sequence DNA amplification from the immune alpaca cDNA and continue through the end of framework 4. The expression in parentheses at the right end indicates that the VHH contains a long hinge (lh) or a short hinge (sh). The locations of each of the three complementarity determining regions (CDRs) are indicated at the top end. FIG. 3 includes the following VHHs containing complementarity determining regions (CDRs), CDR1, CDR2 and CDR3: JIJ B8 (SEQ ID NO: 151) comprises CDR 1: SGSIARPGA (SEQ ID NO: 170); CDR2: SITPGGLTN (SEQ ID NO: 171); and CDR3: HARIIPLGLGSEYRDH (SEQ ID NO: 172); JIK-B8 (SEQ ID NO: 155) comprises CDR 1: ASERSINNYG (SEQ ID NO: 173); CDR2: QISSGGTTN (SEQ ID NO: 174); and CDR3: NSLLRTFS (SEQ ID NO: 175); JKH-A4 (SEQ ID NO: 159) comprises CDR 1: SGLTFGNYA (SEQ ID NO: 176); CDR2: SISRSGSNTW (SEQ ID NO: 177); and CDR3: AGGSYNSDWWNYMY (SEQ ID NO: 178); JHK-C7 (SEQ ID NO: 160) comprises CDR 1: SGRTFSGYA (SEQ ID NO: 179); CDR2: DISWSGHNTY (SEQ ID NO: 180); and CDR3: AEGARTHLSDSYYFPGLWAEPPVGY (SEQ ID NO: 181); JKH-D12 (SEQ ID NO: 161) comprises CDR 1: SGRTFTSYY (SEQ ID NO: 182); CDR2: SIGWTDDNTY (SEQ ID NO: 183); and CDR3: AADYGSGIRAWYNWIY (SEQ ID NO: 184); JKM-A6 (SEQ ID NO: 162) comprises CDR 1: SGATLDTYII (SEQ ID NO: 185); CDR2: CINRSGSTT (SEQ ID NO: 186); and CDR3: AADASYRTCGGSWWNWAY (SEQ ID NO: 187); JKO-A4 (SEQ ID NO: 163) comprises CDR 1: SGFTFSSYT (SEQ ID NO: 188); CDR2: DINGGGDRTD (SEQ ID NO: 189); and CDR3: AKDLSYVSGTYFAND (SEQ ID NO: 190); JKO-B8 (SEQ ID NO: 164) comprises CDR 1: SGIIFDYYSV (SEQ ID NO: 191); CDR2: TITGDGSPN (SEQ ID NO: 192); and CDR3: HAKRTIGTKSEY (SEQ ID NO: 193); JKO-E12 (SEQ ID NO: 165) comprises CDR 1: SRMSFSRRP (SEQ ID NO: 194); CDR2: TISSFGDTTN (SEQ ID NO: 195); and CDR3: NTLLATYA (SEQ ID NO: 196); and JKO-H2 (SEQ ID NO: 166) comprises CDR 1: SGRTFSSYV (SEQ ID NO: 197); CDR2: AISRNGGKTY (SEQ ID NO: 198); and CDR3: AAAVAASAEFVTARSNFYEY (SEQ ID NO: 199).

FIG. 4A and FIG. 4B are amino acid sequences of fusion proteins that contain partial translation products of the two VNAs SEQ ID NOs: 103 and 105. FIG. 4A (SEQ ID NO: 167) shows a fusion protein containing VNA1-PA (SEQ ID NO: 103), and FIG. 4B (SEQ ID NO: 168) shows a fusion protein containing VNA2-PA (SEQ ID NO: 105). The proteins are expressed in E. coli and tested as anthrax antitoxins. Proteins VNA1-PA (SEQ ID NO: 103) and VNA2-PA (SEQ ID NO: 105), respectively, are the same as previously named JKD-11 and JKU-1, respectively, in U.S. provisional application Ser. No. 62/089,949 filed Dec. 10, 2014. Both VNA fusion proteins shown in FIGS. 4A and 4B contain an amino terminal thioredoxin fusion partner and hexahistidine (SEQ ID NO: 169) encoded by the pET32b expression vector. In FIGS. 4A and 4B, the VHH sequences are flanked by E-tag peptides (underlined) and separated by the unstructured spacer ((GGGGS)₃) (SEQ ID NO: 145). The 14 amino acid albumin-binding-peptide (ABP), DICLPRWGCLEWED (SEQ ID NO: 146) described in Nguyen A, et al. (2006) Protein engineering, design & selection: PEDS 19: 291-297 is located at the carboxyl end of the fusion protein, separated from the second E-tag by a GGGGS (SEQ ID NO: 147) spacer.

FIG. 5A-FIG. 5D, respectively, are Meyer-Kaplan survival plots showing percent survival (% survival, ordinate) after BoNT/B toxin exposure of each of four amounts, respectively, subjects as a function of time in days (abscissa) following treatment with a preparation of BoNT/B neutralizing VHH heterodimers as indicated. In FIG. 5A-FIG. 5D, an amount of BoNT/B, toxin of 10, 40, 100 and 500 LD50, respectively, was administered by intraperitoneal injection to groups of five C57BL/6J mice. The mice receiving the toxin were treated with 2 μg of one of BoNT/B neutralizing VHH heterodimers (SEQ ID NO: 131, SEQ ID NO: 133, SEQ ID NO: 135, or SEQ ID NO: 137). Mice were monitored at least five times per day for survival and symptoms of botulism for seven days.

FIG. 6 is a table of the VHH names and binding properties. The first eleven VHHs were obtained by panning using PA83 bound to plastic, after which the K_{D} values for these VHHs were assessed by SPR-PROTEON. The second group of nine VHHs were obtained by panning using 14B7-bound PA83, and the K_{D} values were assessed by SPR-Biacore. The K_{D} for JIK-B8, obtained as an internal reference for SPR-Biacore group, was observed to be 1±0.7. EC₅₀ values were assessed by dilution ELISAs, and IC₅₀s were assessed by toxin neutralization assays on macrophages and competition groups (C-groups) by competition ELISA. N/A refers to antibodies that were observed to not neutralize toxin and thus have no measurable IC₅₀.

FIG. 7A-FIG. 7C are graphs showing the results of competition ELISAs and neutralization assays. Data in FIG. 7A and FIG. 7B were obtained using 14B7- or 2D3-captured PA which was bound to plates and an increasing concentration per plate of each VHH was then added and binding was assessed with an HRP-conjugated anti-Hisx6 or E-tag antibody using standard ELISA protocols. FIG. 7C shows representative neutralization assays for each of the four VHHs representing the four competition groups, a heteromultimer of two neutralizing VHHs, and mAb 14B7, with viability of the ordinate as a function of antibody concentration. All data were obtained from assays are representative of at least two separate repetitions.

### DETAILED DESCRIPTION

Anthrax is a toxigenic disease, which rapidly progresses to lethality for the host if left untreated. The bioterrorist attacks utilizing Bacillus anthracis spores highlighted the need for cost-effective treatments that could be produced on a large scale if necessary. Almost all the therapeutics developed against the disease focus on the anthrax toxins, which have been demonstrated to be the primary virulence determinants. Examples herein describe a novel recombinant anti-toxin consisting of a heterodimer of two camelid anti-anthrax PA heavy chain VHH binding domains as an efficient therapeutic agent. A number of antibodies have been produced against the PA receptor-binding component of the tripartite toxin, and protection in animal models is demonstrated by data herein. Most antibodies target the same epitope of the toxin, which is the dominant neutralizing antigenic region, and only differ in varying affinities and clearance rates.

Anthrax disease is caused by a complex toxin that contains a protective antigen (PA), a lethal factor (LF) and an edema factor (EF). Recombinant engineered proteins as antibodies against PA are described herein which are protective against the disease. Heavy-chain-only Ab V_{H} (VHH) domains with affinity for PA were obtained from immunized alpacas and were screened for anthrax neutralizing activity in macrophage toxicity assays.

Two classes of neutralizing VHHs were identified that recognized distinct and non-overlapping epitopes. One class of VHHs recognized were observed that domain 4 of PA at a neutralizing site that blocks PA binding to cells. Another class of VHHs recognized a novel, conformational epitope. A VHH antibody described herein was observed to inhibit conversion of the PA63 oligomer from “pre-pore” conformation to a SDS and heat-resistant “pore” conformation. The antibody described herein was observed to prevent endocytosis of cell surface generated PA63 subunit. The monomer neutralizing VHHs administered at 2:1 molar ratio to PA were observed to be effective in protecting mice from a lethal anthrax toxin challenge. The highest affinity members of different anti-PA VHH classes were expressed as two heterodimeric VHH-based neutralizing agents (VNAs). VNAs were observed to have improved neutralizing potency in cell assays and to have protected mice from anthrax toxin challenge with better efficacy than their corresponding monomer VHHs. The VNA2-PA (JKU-1) which was observed to be most efficient consists of a heterodimer of the novel oligomer-inhibiting VHH (JKH-C7) and a receptor blocking VHH (JIK-B8). This VNA2-PA was observed to protect mice against toxin challenge at 1:1 molar ratio to toxin and increased survival times were observed at submolar ratios. Furthermore, the antibody also provided protection against A35 spore challenge. VNA2-PA (JKU-1) has potential as an anthrax therapeutic, and its simple and stable nature is amenable to administration by genetic delivery or by respiratory routes.

The novel VHH-based VNA described herein consists of two anti-toxin VHHs targeting independent epitopes of PA and inhibiting the action of the toxin at two different functional steps. The VHH based VNA agent described herein is more effective in vivo by a factor of at least about 20-50 fold compared to the well-characterized neutralizing antibody 14B7 which acts on the same epitope as the approved human anti-PA antibody, RAXIBACUMAB (Abthrax) in protecting against anthrax toxin challenge and spore infection. The affinity is 0.07 nM in contrast to the 2.78 nM affinity of Abthrax, a commercially available monoclonal antibody, RAXIBACUMAB, that neutralizes toxins produced by B. anthracis (Human Genome Sciences, Rockville, MD).

An antitoxin strategy herein uses VNAs consisting of two or more, linked, toxin neutralizing, VHHs recognizing non-overlapping epitopes on PA. An advantage of covalently linking VHHs together is a resulting increased toxin binding affinity and increase in potency of neutralization through targeting of two different steps in the interaction of the toxin with cells. A benefit of the conformational epitope of the VNA, JKH-C7 arm is the extremely low likelihood of easily circumventing the PA-antibody interaction through a small number of mutations in genes encoding PA. The bulk of previously available anti-PA neutralizing antibodies target the same receptor-binding epitope that the JIK-B8 arm of the VNA targets, and the receptor-binding epitope can be destroyed by genetic manipulation of the PA antigen to eliminate reactivity with these neutralizing antibodies. The complex conformational epitope for the JKH-C7 VHH arm of the antibody described herein is unlikely to be easily disrupted without impact on PA function.

The presence of toxins in the circulation causes a wide variety of human and animal illnesses. Antitoxins are therapeutic agents that prevent toxin infection or reduce further development of negative symptoms in patients that have been exposed to a toxin (a process referred to as “intoxication”). Typically, antitoxins are antisera obtained from large animals (e.g., sheep, horse, and pig) that were immunized with inactivated or non-functional toxin. More recently, antitoxin therapies have been developed using combinations of antitoxin monoclonal antibodies including yeast-displayed single-chain variable fragment antibodies generated from vaccinated humans or mice. See Nowakowski et al. 2002. Proc Natl Acad Sci USA 99: 11346-11350; Mukherjee et al. 2002. Infect Immun 70: 612-619; Mohamed et al. 2005 Infect Immun 73: 795-802; Walker, K. 2010 Interscience Conference on Antimicrobial Agents and Chemotherapy—50th Annual Meeting—Research on Promising New Agents: Part 1. IDrugs 13: 743-745. Antisera and monoclonal antibodies are difficult to produce economically at scale, usually requiring long development times and resulting in problematic quality control, shelf-life and safety issues. New therapeutic strategies to develop and prepare antitoxins are needed.

Antitoxins function through two key mechanisms, neutralization of toxin function and clearance of the toxin from the body. Toxin neutralization occurs through biochemical processes including inhibition of enzymatic activity and prevention of binding to cellular receptors. Antibody mediated serum clearance occurs subsequent to the binding of multiple antibodies to the target antigen (Daeron M. 1997 Annu Rev Immunol 15: 203-234; Davies et al. 2002 Arthritis Rheum 46: 1028-1038; Johansson et al. 1996 Hepatology 24: 169-175; and Lovdal et al. 2000 J Cell Sci 113 (Pt 18): 3255-3266). Multimeric antibody decoration of the target is necessary to permit binding to the Fc receptors which have only low affinity (Davies et al. 2002 Arthritis Rheum 46:1028-1038 and Lovdal et al. 2000 J Cell Sci 113 (Pt 18): 3255-3266). Without being limited by any particular theory or mechanism of action, it is here envisioned that an ideal antitoxin therapeutic would both promote toxin neutralization to immediately block further toxin activity and would also accelerate toxin clearance to eliminate future pathology if neutralization becomes reversed.

Effective clearance of botulinum neurotoxin (BoNT), a National Institute of Allergy and Infectious Diseases (NIAID) Category A priority pathogen, is believed by some researchers to require three or more antibodies bound to the toxin. Nowakowski et al. 2002. Proc Natl Acad Sci USA 99: 11346-11350 determined that effective protection of mice against high dose challenge of BoNT serotype A (BoNT/A) requires co-administration of three antitoxin monoclonal antibodies, and that all three antibodies presumably promote clearance. Administration of a pool of three or more small binding agents, each produced with a common epitopic tag, reduced serum levels of a toxin when co-administered with an anti-tag monoclonal antibody (Shoemaker et al. U.S. published application 2010/0278830 A1 published Nov. 4, 2010 and Sepulveda et al. 2009 Infect Immun 78: 756-763, each of which is incorporated herein in its entirety). The tagged binding agents directed the binding of anti-tag monoclonal antibody to multiple sites on the toxin, thus indirectly decorating the toxin with antibody Fc domains and leading to clearance of the toxin through the liver.

Pools of scFv domain binding agents with specificity for BoNT/A and each containing a common epitopic tag (E-tag), had been shown to be effective for decorating the botulinum toxin with multiple anti-tag antibodies (Shoemaker et al. U.S. utility patent publication number 2010/0278830 published Nov. 4, 2010 and U.S. continuation-in-part patent publication number 2011/0129474 published Jun. 2, 2011, each of which is incorporated herein by reference in its entirety). Administration of binding agents and clearance antibodies to subjects resulted in clearance via the liver with an efficacy in mouse assays equivalent to conventional polyclonal antitoxin sera. Ibid. and Sepulveda et al. 2009 Infect Immun 78: 756-763. The tagged scFvs toxin targeting agents and the anti-tag monoclonal antibodies were effective to treat subjects at risk for or having been contacted with a disease agent.

The use of small binding agents to direct the decoration of toxin with antibody permits new strategies for the development of agents with improved therapeutic and commercial properties. Examples herein show that a single recombinant heterodimeric binding protein/agent which contains two or more high-affinity BoNT binding agents (camelid heavy-chain-only Ab VH (VHH) domains) and two epitopic tags, co-administered with an anti-tag mAb, protected subjects from negative symptoms and lethality caused by botulism. Further, the binding protein was observed to have antitoxin efficacy equivalent to and greater than conventional BoNT antitoxin serum in two different in vivo assays. Examples herein compare neutralizing or non-neutralizing binding agents administered with or without clearing antibody, and show the relative contributions of toxin neutralization and toxin clearance to antitoxin efficacy. Examples herein show that both toxin neutralization and toxin clearance contribute significantly to antitoxin efficacy in subjects. Toxin neutralization or toxin clearance using heterodimer binding protein antitoxins was observed herein to sufficiently protect subjects from BoNT lethality in a therapeutically relevant, post-intoxication assay. Methods in further Examples herein include an optional clearing antibody for example a monoclonal anti-E-tag antibody.

It was observed in Examples herein that VHH binding agents that neutralized toxin function significantly improved the antitoxin efficacy and even obviated the need for clearing antibody in a clinically relevant post-intoxication BoNT/A assay.

### Pharmaceutical Compositions

An aspect of the present invention provides pharmaceutical compositions, wherein these compositions comprise an antigen from a toxin of B. anthracis or C. botulinum peptide or protein, and optionally further include an adjuvant, and optionally further include a pharmaceutically acceptable carrier. In various embodiments, the compositions include at least one atoxic protein or a source of expression of the protein, such that the protein elicits an immune response specific for a B. anthracis or C. botulinum toxin.

In certain embodiments, these compositions optionally further comprise one or more additional therapeutic agents. In certain embodiments, the additional therapeutic agent or agents are selected from the group consisting of antibiotics particularly antibacterial compounds, anti-viral compounds, anti-fungals, and include one or more of growth factors, anti-inflammatory agents, vasopressor agents, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGF), epidermal growth factor (EGF), platelet derived growth factor (PDGF), neu differentiation factor (NDF), hepatocyte growth factor (HGF), and hyaluronic acid.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's The Science and Practice of Pharmacy Ed. by LWW 21^{st} EQ. PA, 2005 discloses various carriers used in formulating pharmaceutical compositions and known techniques for the preparation thereof. Carriers are selected to prolong dwell time for example following any route of administration, including IP, IV, subcutaneous, mucosal, sublingual, inhalation or other form of intranasal administration, or other route of administration.

Some examples of materials that can serve as pharmaceutically acceptable carriers include, but are not limited to, sugars such as glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In yet another aspect, according to the methods of treatment of the present invention, the immunization is promoted by contacting the subject with a pharmaceutical composition, as described herein. Thus, the invention provides methods for immunization comprising administering a therapeutically effective amount of a pharmaceutical composition comprising active agents that include an immunogenic toxin protein of B. anthracis or C. botulinum to a subject in need thereof, in such amounts and for such time as is necessary to achieve the desired result. It will be appreciated that this encompasses administering an inventive vaccine as described herein, as a preventive or therapeutic measure to promote immunity to infection by B. anthracis or C. botulinum, to minimize complications associated with the slow development of immunity (especially in compromised patients such as those who are nutritionally challenged, or at risk patients such as the elderly or infants).

In certain embodiments of the present invention a “therapeutically effective amount” of the pharmaceutical composition is that amount effective for promoting production of antibodies and activity in serum specific for the toxins of B. anthracis or C. botulinum, or disappearance of disease symptoms, such as amount of antigen or toxin or bacterial cells in feces or in bodily fluids or in other secreted products. The compositions, according to the method of the present invention, may be administered using any amount and any route of administration effective for generating an antibody response. Thus, the expression “amount effective for promoting immunity”, as used herein, refers to a sufficient amount of composition to result in antibody production or remediation of a disease symptom characteristic of infection by B. anthracis or C. botulinum.

The exact dosage is chosen by the individual physician in view of the patient to be treated. Dosage and administration are adjusted to provide sufficient levels of the active agent(s) or to maintain the desired effect. Additional factors which may be taken into account include the severity of the disease state; contact to infectious agent in the past or potential future contact; age, weight and gender of the patient; diet, time and frequency of administration; drug combinations; reaction sensitivities; and tolerance/response to therapy. Long acting pharmaceutical compositions might be administered every three to four days, every week, or once every two weeks depending on half-life and clearance rate of the particular composition.

The active agents of the invention are preferably formulated in dosage unit form for ease of administration and uniformity of dosage. The expression “dosage unit form” as used herein refers to a physically discrete unit of active agent appropriate for one dose to be administered to the patient to be treated. It will be understood, however, that the total daily usage of the compositions of the present invention will be decided by the attending physician within the scope of sound medical judgment. For any active agent, the therapeutically effective dose can be estimated initially either in cell culture assays or in animal models, usually mice, rabbits, dogs, or pigs or piglets or other suitable animals. The animal models described herein including that of chronic or recurring infection by B. anthracis or C. botulinum is also used to achieve a desirable concentration range and route of administration. Such information can then be used to determine useful doses and routes for administration in humans.

A therapeutically effective dose refers to that amount of active agent which ameliorates at least one symptom or condition. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred. The data obtained from cell culture assays and from animal studies are used in formulating a range of dosage for human use.

The therapeutic dose shown in examples herein is at least about 1 μg per kg, at least about 5, 10, 50, 100, 500 μg per kg, at least about 1 mg/kg, 5, 10, 50 or 100 mg/kg body weight of the purified toxin vaccine per body weight of the subject, although the doses may be more or less depending on age, health status, history of prior infection, and immune status of the subject as would be known by one of skill in the art of immunization. Doses may be divided or unitary per day and may be administered once or repeated at appropriate intervals.

### Administration of Pharmaceutical Compositions

After formulation with an appropriate pharmaceutically acceptable carrier in a desired dosage, the pharmaceutical compositions of this invention can be administered to humans and other mammals topically (as by powders, ointments, or drops), orally, rectally, mucosally, sublingually, parenterally, intracisternally, intravaginally, intraperitoneally, bucally, sublingually, ocularly, or intranasally, depending on preventive or therapeutic objectives and the severity and nature of a pre-existing infection.

In various embodiments of the invention herein, it was observed that high titers of antibodies, sufficient for protection against a lethal dose of B. anthracis or C. botulinum toxin, were produced after administration of the engineered atoxic toxin proteins provided herein. Liquid dosage forms for oral administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of an inventive pharmaceutical composition include ointments, pastes, creams, lotions, gels, powders, solutions, sprays, inhalants, or patches. The active agent is admixed under sterile conditions with a pharmaceutically acceptable carrier and any needed preservatives or buffers as may be required. Administration may be therapeutic, or it may be prophylactic.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized prior to addition of spores, for example, by filtration through a bacterial-retaining filter, or by incorporating sterilizing agents in the form of sterile solid compositions which can be dissolved or dispersed in sterile water or other sterile injectable medium prior to use. In order to prolong the effect of an active agent, it is often desirable to slow the absorption of the agent from subcutaneous or intramuscular injection. Delayed absorption of a parenterally administered active agent may be accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly (orthoesters) and poly (anhydrides). Depot injectable formulations are also prepared by entrapping the agent in liposomes or microemulsions which are compatible with body tissues.

Compositions for rectal or vaginal administration are preferably suppositories which can be prepared by mixing the active agent(s) of this invention with suitable non-irritating excipients or carriers such as cocoa butter, polyethylene glycol or a suppository wax which are solid at ambient temperature but liquid at body temperature and therefore melt in the rectum or vaginal cavity and release the active agent(s).

Solid dosage forms for oral, mucosal or sublingual administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active agent(s) only, or preferentially, in a certain part of the intestinal tract, optionally, in a delayed manner. Examples of embedding compositions which can be used include polymeric substances and waxes.

### Substantially Identical Amino Acid and Nucleotide Sequences for VHHs

There is a large body of information in the literature supporting the fact that closely related antibody (Ab) sequences are capable of performing the same binding and therapeutic functions such that this is now generally accepted by those with ordinary skill in immunological sciences and is even a dogma. The creation of Abs with small numbers of amino acid sequence variations occurs naturally within mammals and other some other animal species during the process of ‘affinity maturation’ in which cells producing Abs that bind a newly encountered antigen (Ag) are expanded such that progeny cells contain random mutations within portions of the Ab coding DNA that results in new, related Ab sequences. The cells expressing Abs that have gained improved binding properties for the new Ag are then selected and expanded, increasing the amount of the improved antibody in the animal. This process continues through multiple generations of mutation and selection until Abs with greatly improved binding properties result, thus providing, for example, better immunity against pathogens possessing the new Ag. This process of Ab affinity maturation is widely accepted in the literature and clearly demonstrates that related Ab amino acid sequences can possess similar target binding properties and perform similar therapeutic functions in vivo.

In examples herein, there are numerous examples of related Ab sequences performing similar functions and providing similar therapeutic benefits. The Abs described herein are mostly heavy-chain only Abs (HcAbs) from Camelids. The Vu region from the DNA is isolated encoding these Abs and expressed as single-domain Abs called VHHs. Alpacas are immunized with a selected Ag multiple times to permit the animal to undergo affinity maturation of the HcAbs they produce recognizing this Ag. The HcAbs are then isolated and the DNA encoding the VHH regions are closed for expression of soluble VHHs that bind the Ag and have potential therapeutic or diagnostic properties. During this process, many examples of closely related VHHs are isolated presumably different which are intermediates resulting from the alpacas' affinity maturation process. These related VHHs are screened and most promising members of each homology group is identified, and becomes a lead candidate for further development. VHHs, like all mammalian antibodies, consist of four well-conserved ‘framework’ regions (FRs) which are important to form the antibody structure. Between the FRs (FR1, FR2, FR3 and FR4) are three much less well-conserved ‘complementarity determining regions’ or CDRs which form the interactions with the Ags. These binding regions must bind to widely varying structures (epitopes) on different Ags, therefore, the CDRs must also vary widely so as to interact and bind to these Ags. The third CDR, CDR3, is generally the longest and most diverse of the CDRs within VHHs, both in size and sequence. CDR3 in VHHs can range in size from about 7 to about 28 amino acid residues [1]. The CDR3 regions of VHHs from the same alpacas selected for their binding to a common target Ag, prove to be very similar in their size and have many amino acid identities; the chance that this occurred by random chance are astronomical. Therefore, these VHHs resulted from affinity maturation of a common precursor VHH within the animal and are classified as being a ‘homology group’. The individual VHHs within a homology group are classified for binding to a target the members of the VHH homology group ‘compete’ with each other for binding, thus demonstrating that they bind to the same region on the target.

Since the FRs are critical for sustaining the structure of the VHH and the positioning of the CDRs for binding to their target Ag, the FRs must not vary too much in sequence. Some variation, particular when replacement amino acids are related in properties, is permissible and these changes can often be found naturally within VHHs that have undergone affinity maturation in an animal. In addition to the FRs, the CDRs also must not vary too much in sequence or their Ag binding affinity will be compromised. An excellent way to estimate how much amino acid sequence variation is tolerated within VHHs without compromising their Ag binding character is to observe the variation that occurs naturally within affinity matured homology groups of VHHs isolated from the same animals and shown to bind to the same Ag.

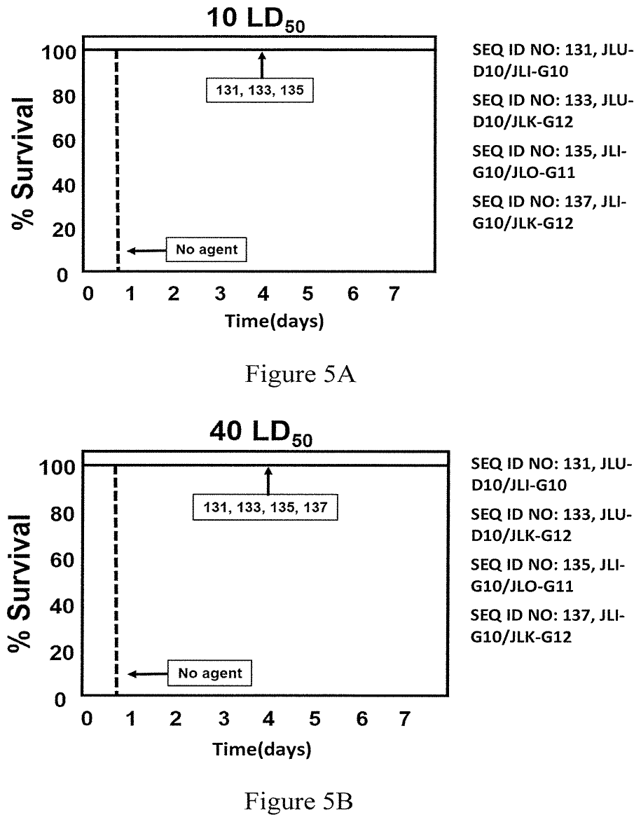

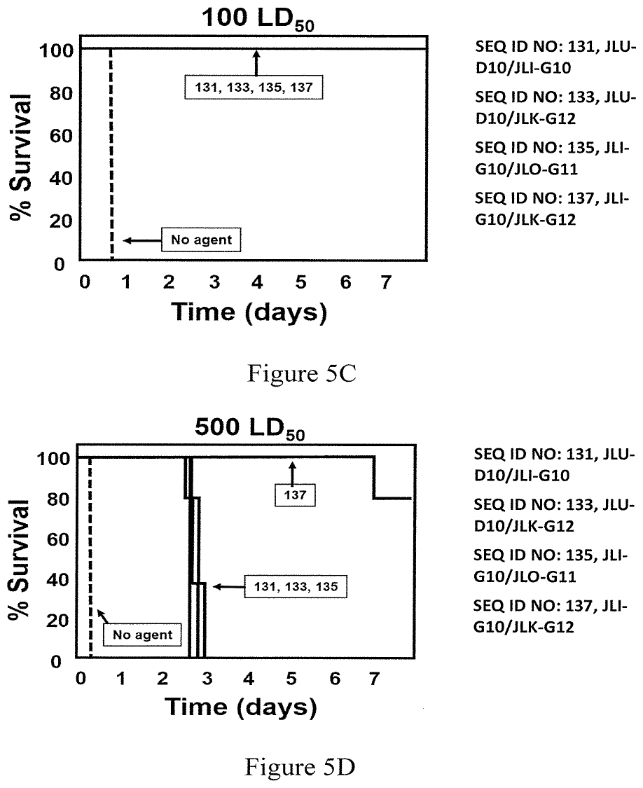

An example of VHH sequence relatedness necessary to retain common Ag binding properties is described in U.S. Pat. No. 8,349,326, issued Jan. 8, 2014 and represented in FIG. 5. In this example, the substantial identity of five different VHH sequences shown in the patent, JDO-E9, JDQ-B2, JDQ-B5, JDQ-C5, and JDQ-F9 is represented as a phylogenic tree. These sequences are substantially different from each other and form a clear homology group when their sequences are compared to the sequences of seven random VHHs. All five members of this homology group had been selected for their binding to Botulinum neurotoxin serotype A (BoNT/A), all had clearly related CDR3 regions, and all were found to compete with each other for binding to BoNT/A. Therefore, these sequences had a common binding site. Despite their common clonal origin and common Ag binding sites, these VHHs of 108 amino acid length contained as many as 26 amino acid differences. This implied that VHHs containing up to 24% amino acid sequence variation had retained their ability to bind to the same region of BoNT/A.

Another example that describes acceptable amount of VHH sequence variation within related VHHs having the same Ag binding character is described in Tremblay et al., 2013 Infect Immun 81: 4592-4603. Proteins in large homology group are described containing 11 VHH sequences, Stx-A3, A4, A5, D4, F1, G6, H3, H5, H9, H10, and H12 with closely related CDR3 sequences of identical size, and the unusual property of cross-specific binding to two different Shiga toxins, Stx1 and Stx2. Two of the more distantly related members of this homology group, VHHs Stx-A4, Stx-A5 are characterized as having common Ag binding character. These two related VHHs have 32 amino acid changes in their full 120 or 121 residue VHH sequence. Therefore, 26% amino acid variation in sequence does not result in the loss of their common Ag binding property.

A portion of the data herein was published as follows, “Prolonged prophylactic protection from botulism with a single adenovirus treatment promoting serum expression of a VHH-based antitoxin protein” by co-authors Mukherjee J, Dmitriev I, Debatis M, Tremblay J M, Beamer G, Kashentseva E A, Curiel D T, Shoemaker C B, in the journal PLoS ONE 9 (8): e106422 2014 August doi:10.1371/journal.pone.0106422; “Adenovirus vector expressing Stx1/2-neutralizing agent protects piglets infected with E. coli 0157:H7 against fatal systemic intoxication” by co-authors Sheoran A S, Dmitriev I P, Kashentseva E A, Cohen O, Mukherjee J, Debatis M, Shearer J, Tremblay J M, Beamer G, Curiel D T, Shoemaker C B, Tzipori S, in the journal Infect Immun. 2014 Nov. 3. pii: IAI.02360-14; and “A heterodimer of a VHH (variable domains of camelid heavy chain-only) antibody that inhibits anthrax toxin cell binding linked to a VHH antibody that blocks oligomer formation is highly protective in an anthrax spore challenge model” by co-authors Moayeri M, Leysath C E, Tremblay J M, Vrentas C, Crown D, Leppla S H, Shoemaker C B, in the journal J Biol Chem. 2015 Mar. 6; 290(10):6584-95 which appeared online Jan. 6, 2015. These papers are hereby incorporated in their entireties herein. The invention now having been fully described, it is further exemplified by the following claims.

### EXAMPLES

### Example 1: Toxins and Spores

Endotoxin-free mutant PA proteins, including wild type PA83, PA63, and LF were purified from B. anthracis as described in Park, S., et al., 2000 Protein expression and purification 18, 293-302. The PAΔΔ is a mutant from which amino acid residues at positions 162-167 and 304-317 of the amino acid sequences have been genetically deleted, such that the protein cannot be cleaved by furin and accumulates on the cell surface. PAdFF is a mutant in which phenylalanine residues at positions 313 and 314 have been deleted thereby making the protein unable to translocate LF and EF (Singh, Y., et al., 1994 The Journal of biological chemistry 269, 29039-29046). Concentrations of LT correspond to the concentration of each toxin component (i.e. 1 μg/mL LT is 1 μg/mL PA+1 μg/mL LF). Spores of the non-encapsulated, toxigenic Sterne-like strain A35 (Pomerantsev, A. P., et al., 2006 Infection and immunity 74, 682-693) used to infect mice were prepared as described in Moayeri, M., et al., 2010 PLoS pathogens 6, e1001222.

### Example 2: Reagents

Rabbit anti-PA83 polyclonal serum #5308 and neutralizing anti-PA mouse monoclonal antibody (mAb) 14B7, which blocks binding of PA (both PA83 and PA63) to its cellular receptors was manufactured as described in Rosovitz, M. J., et al., 2003 The Journal of biological chemistry 278, 30936-30944. Antibodies against the N-terminus of MEK1 (Calbiochem-EMD Biosciences, San Diego, CA), horse radish peroxidase (HRP)-conjugated and non-conjugated anti-E-tag polyclonal antibodies (Bethyl Labs, Montgomery, TX) and various IR-dye tagged secondary antibodies (Rockland Labs, Boyertown, PA) were purchased. The dye 3-(4,5-dimethyl-2-thiazolyl)-2,5-diphenyl tetrazolium bromide (MTT) was purchased from Sigma (St. Louis, MO).

### Example 3: VHH-Display Library Preparation from Genes Expressed in Immunized Alpacas

Three alpacas were immunized with PA83 (100 μg) by five successive multi-site subcutaneous (SC) injections at three week intervals. For the first immunization, the adjuvant was alum/CpG and subsequent immunizations used alum. All alpacas achieved ELISA anti-PA titers of 1:1,000,000. Blood was obtained from the alpacas for lymphocyte preparation seven days after the fifth immunization, and RNA was extracted using the RNEASY kit (Qiagen, Valencia, CA). Two VHH-display phage libraries were prepared as described in Maass, D. R., et al., 2007 International journal for parasitology 37, 953-962 and Tremblay, J. M., et al., 2010 Toxicon 56, 990-998. The forward and reverse primers used to amplify the VHH coding region repertoire contained Not1 and Asc1 sites, which were used to ligate the JSC vector for gene III phage display. The first library (JIG-2) was constructed using RNA obtained from peripheral blood lymphocytes (PBLs) of one immunized alpaca and contained about 1×10⁷ independent clones, and the second library (JKF-1) was generated from RNA obtained from a pool of PBLs of the other two alpacas, and contained about 3×10⁷ independent clones.

### Example 4: ELISAs

Purified VHH preparations were serially diluted onto ELISA plates coated with 1 μg/ml of each of the different PA proteins, incubated for one hour at room temperature, washed and then incubated for one hour with HRP-anti-E-tag. Bound HRP was detected using 3,3′,5,5′-tetramethylbenzidine (Sigma) and values were plotted as a function of the input VHH concentration. EC₅₀ values were calculated for the VHH concentration that secreted in a signal equal to 50% of the maximum signal.

### Example 5: Anti-PA VHH Identification and Preparation

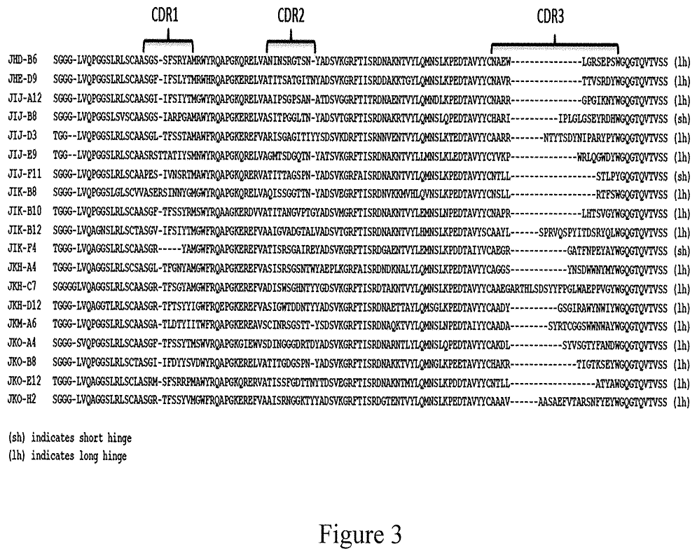

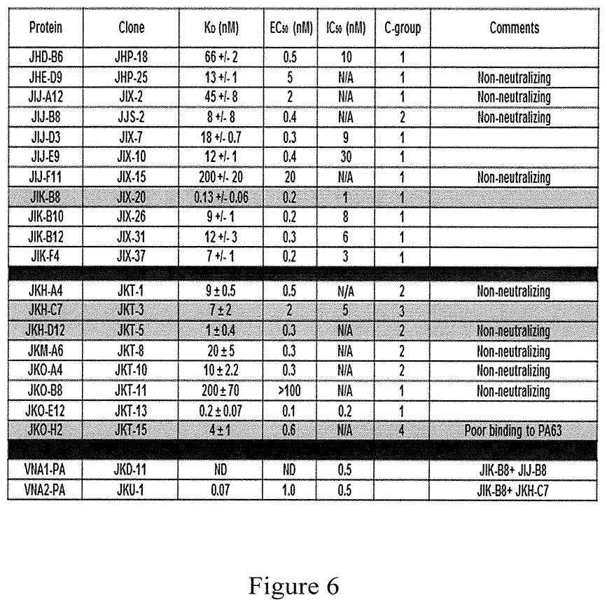

Phage library panning, phage recovery and clone fingerprinting were performed as described in Mukherjee, J., et al., 2012 PLoS ONE 7, e29941, Maass, D. R., et al., 2007 International journal for parasitology 37, 953-962 and Tremblay, J. M., et al., 2010 Toxicon 56, 990-998, as follows. The first panning process utilized the JIG-2 VHH-display library and employed purified PA83 or PA63 coated onto Nunc Immunotubes at 10 μg/ml for the first low stringency pan and 1 μg/ml for the second high stringency pan. After two panning cycles, 70% of random clones selected on each target produced a signal two-fold greater than background. The clones that produced strongest ‘bug supernatant’ ELISA (Tremblay, J. M., et al., 2013 Infection and immunity 81, 4592-4603) signals on plates coated with 0.5 μg/ml PA83 were fingerprinted. The VHHs that had been panned on PA83 or PA63 were observed to recognize both PA83 and PA63. VHH coding sequences were determined for 24 clones displaying clear unique fingerprints (Tremblay, J. M., et al., 2013 Infection and immunity 81, 4592-4603). Sequence alignments showed 11 distinct homology groups. Amino acid sequences of clones representing each group are shown in FIG. 3. A second panning process using the JKF-1 library was performed similar to the first panning process and PA83 was used as the target. About 300 colonies were picked randomly and screened by bug supernatant ELISA on replica plates coated with either 0.5 μg/ml PA, or 3 μg/ml 14B7 mAb (Little, S. F., et al., 1988 Infection and immunity 56, 1807-1813) followed by 1 μg/ml PA83. Screening on 14B7-captured PA83 was performed to block binding of VHHs recognizing the dominant epitope (C-group 1, FIG. 6) that was identified following the screening of the first library. About 70% of clones recognized PA83 and about 20% recognized 14B7-captured PA83. From data obtained by ELISA and DNA fingerprinting, about 70 different VHH coding sequences were obtained. The alignment of these 70 different VHH coding sequences led to the identification of eight new homology groups not previously identified in the first screen (VHHs: JKH-A4, JKH-C7, JKH-D12, JKM-A6, JKO-A4, JKO-B8, JKO-E12, AND JKO-H12 in FIGS. 3 and 6). At least one VHH from each homology group was selected for protein expression. Expression and purification of VHHs in E. coli as recombinant thioredoxin (Trx) fusion proteins containing hexahistidine (SEQ ID NO: 169) was performed as previously described in Tremblay, J. M., et al., 2010 Toxicon 56, 990-998. VHH heterodimers were genetically engineered to be linked by a 15-amino acid flexible spacer ((GGGGS)₃) (SEQ ID NO: 145). All VHHs were expressed with a carboxyl-terminal E-tag epitope. Competition ELISA analysis was performed as previously described, with minor modifications (Mukherjee, J., et al., 2012 PLoS ONE 7, e29941).

### Example 6: Affinity Analyses

The kinetic parameters of the VHHs were assessed by performing surface plasmon resonance, using either a PROTEON XPR36 Protein Interaction Array System (Bio-Rad, Hercules, CA; VHHs: JHD-B6, JHE-D9, JIJ-A12, JIJ-B8, JIJ-D3, JIJ-E9, JIJ-F11, JIK-B8, JIK-B10, JIK-B12, and JIK-F4 in FIG. 6) or a BIACORE 3000 (GE Healthcare; VHHs: JKH-A4, JKH-C7, JKH-D12, JKM-A6, JKO-A4, JKO-B8, JKO-E12, and JKO-H12 in FIG. 6). In each assay, the VHH was immobilized to the chip (GLH for PROTEON, CM5 for BIACORE) by amine coupling chemistry, involving sequential activation of the chip surface with a mixture of 1-ethyl-3-(3-dimethylaminopropyl)carbodiimide (EDC) and sulfo-N-hydoxysuccinimide (sulfo-NHS), injection of PA83 at pH 5 (sodium acetate buffer), and deactivation with an ethanolamine injection.

For the PROTEON data set, a range of PA concentrations was passed over the chip surface at 100 μL/min for 60 s, and dissociation was recorded for 600 s or 1200 s. Running buffer for these assays was 10 mM Hepes, pH 7.4, 150 mM NaCl, 0.005% Tween-20. The surface was regenerated between runs with a 30 s injection of 50 mM HCl at 50 μL/min. Data were evaluated with PROTEON Manager software (version 3.1.0.6) using the Langmuir interaction model to obtain K_{D} values. Reported values are the mean of at least four replicates.

For the BIACORE data set, VHHs were passed over the PA immobilized on the chip surface at 100 nM and 100 μl/min for 60 s, and dissociation was recorded for 600 s or 1200 s. Running buffer for these assays was 10 mM HEPES, pH 7.4, 150 mM NaCl, 0.005% Tween-20. The surface was regenerated between runs with a 30 s injection of 10 mM glycine (pH 3) at 50 μl/min. Dissociation and association phases of each curve were fit separately using BIAevaluation software (GE) using the 1:1 Langmuir model to obtain K_{D} values. Reported values are the mean of three replicates. A series of four replicates at 100 nM through 2 μM JKO-B8 resulted in comparable K_{D} values at each concentration. A negative control VHH (anti-EF) did not exhibit any binding to the PA-coated chip. JIK-B8 was run at the beginning and end of the series to provide a point of comparison to the PROTEON data set.

### Example 7: Toxicity and Neutralization Assays

RAW264.7 mouse macrophages were grown in Dulbecco's modified Eagle medium (DMEM) supplemented with 10% fetal bovine serum, 10 mM HEPES, and 50 μg/mL gentamicin (all purchased from Life Technologies, Grand Island, NY). For neutralization assays PA83 and LF (250 ng/ml) in serum-free Dulbecco's Modified Eagle Medium were incubated with each of various dilutions of antibody in 96-well plates for one hour prior to addition to RAW264.7 macrophages. Viability was assessed by MTT staining as described in Chen, Z., et al., 2009 Infection and immunity 77, 3902-3908, at a time point when greater than 90% of toxin-treated controls were observed to be lysed by assessment by light microscopy. In certain experiments PA83 or PA63 (1 μg/ml) were pre-bound to antibodies or were added to cells at 37° C. or 4° C. followed after one hour by washing with serum-free DMEM at the same temperature and addition of medium containing LF or antibodies prepared in LF (1 μg/ml). Cells were then incubated at 37° C. for 12-16 hours. Viability was then assessed by MTT staining relative to untreated cell controls.

### Example 8: Mouse Studies

For toxin challenge, Balb/cJ mice (female, 8 weeks old, Jackson Laboratories, Bar Harbor, ME) were treated with antibody agents by the IV route at the doses (molar ratios relative to PA) and times described in brief description of the figures. Mice were challenged with LT (45 μg, IV) and monitored for 10 days for survival. For spore challenges, C57BL/6J mice (8 weeks old, female, Jackson Laboratories) were challenged with the lethal dose of 2×10⁷ spores (SC, 200 μl) before or after antibody administration (SC) at various doses and times as noted in brief description of the figures.

### Example 9: Ethics Statement

All examples were performed under protocols approved by Tufts University and National Institute of Allergy and Infectious Diseases (NIAID) Animal Care and Use Committees. Work with alpacas was performed at Tufts under approved protocol Tuskegee University School of Veterinary Medicine (TUSVM) and Institutional Animal Care and Use Committee (IACUC) Protocol #G2011-08. Mouse studies were performed at NIAID under approved protocols LPD8E and LPD9E.

### Example 10: Anthrax PA-Binding VHHs

VHH-display phage libraries were prepared from genetic material obtained from three alpacas, which had been immunized with purified anthrax PA83. Two separate libraries were selected for clones binding to PA83 or, to PA83 immobilized on mAb 14B7. The mAb 14B7 is a well-characterized neutralizing mAb that binds to an immunodominant epitope through which PA binds to its receptor.

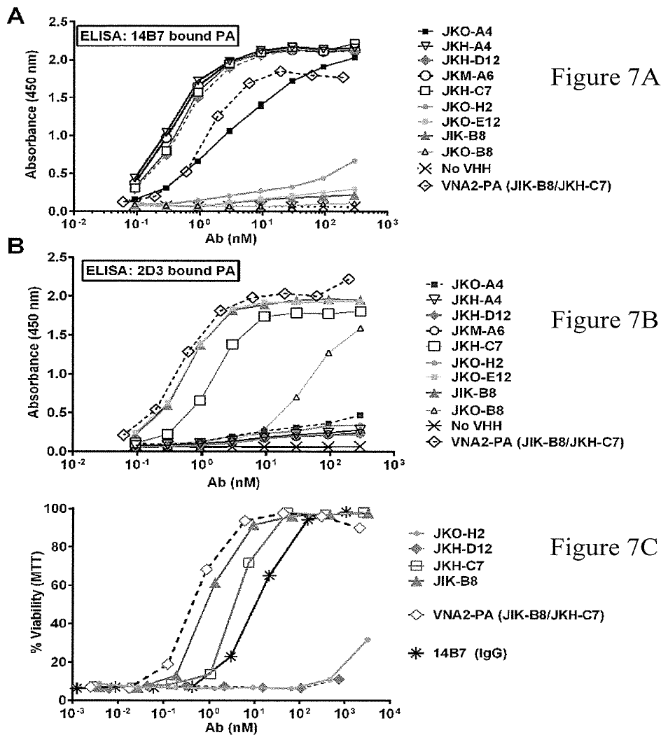

Of total clones obtained and sequenced, 19 VHHs with apparently unrelated sequences were identified (FIG. 3). Competition assays between the various VHHs, and with 14B7 and 2D3 mAbs, which bind distinct immunodominant regions of PA83 (Little, S. F., et al., 1988 Infection and immunity 56, 1807-1813) showed that the identified VHHs fall into four distinct competition groups (identified as 1, 2, 3, and 4 in FIG. 6) and thus likely each protein in a group binds to one of four non-overlapping epitopes on PA. Ten of the 11 VHHs selected by binding to PA83-coated tubes (VHHs: JHD-B6, JHE-D9, JIJ-A12, JIJ-B8, JIJ-D3, JIJ-E9, JIJ-F11, JIK-B8, JIK-B10, JIK-B12, and JIK-F4 in FIG. 6) competed with 14B7. Eight unique PA-binding VHHs, including six that bind PA83 at sites different than 14B7 (FIG. 7A and FIG. 7B) were subsequently selected by binding to 14B7-immobilized (and thereby blocked) PA (VHHs: JKH-A4, JKH-C7, JKH-D12, JKM-A6, JKO-A4, JKO-B8, JKO-E12, and JKO-H12 in FIG. 6). Binding of one of the VHH, clone JIJ-B8 was not blocked by either mAb, and binding of another clone JKO-H2 was inhibited by both mAbs (FIG. 7A and FIG. 7B). The VHHs were characterized for PA affinity by dilution ELISA (for EC₅₀) and by surface plasmon resonance (for K_{D}) (FIG. 6). A selected VHH representative of each of the four epitope competition groups is illustrated by a shaded portion in FIG. 6. These are JIK-B8 (C-group 1), JKH-C7 (C-group 3), JKH-D12 (C-group 2), and JKO-H2 (C-group 4).

### Example 11: Anthrax Toxin Neutralization

Cell-based anthrax toxin neutralization assays were performed on each of the 19 unique VHHs, and the data showed potencies ranging from IC₅₀ of about 200 μM to no activity in an assay using PA at 1.25 nM (FIG. 6; representative assay with antibodies from each competition group shown in FIG. 7C). VHHs recognizing the immunodominant PA domain (group 1) differed widely in their ability to neutralize the toxin, with four of 12 showing no neutralizing ability. VHHs JIK-B8 and JKO-E12 of the C-group 1 class displayed the highest affinity and lowest IC₅₀ values. One VHH recognized a second epitope (JKH-C7, group 3, FIG. 6) showed potent anthrax neutralizing activity (FIG. 7C). VHHs that had been characterized as recognizing C-group 2 and C-group 4 showed weak or undetectable toxin neutralizing activity (FIG. 7C). VHH JKO-H2 (group 4) displayed no recognition of PA63, suggesting that furin cleavage either removes the epitope or alters it in a manner that it cannot be recognized.

### Example 12: Heterodimeric VHH-Based Neutralizing Agents (VNAs) Protect Against Anthrax Toxin and Spore Infection in Mice

Linking toxin-neutralizing VHHs into heteromultimeric VNAs has been found to improve toxin affinity and, more importantly, to substantially improve in vivo antitoxin efficacy (Mukherjee, J., et al., 2012 PLoS ONE 7, e29941; Tremblay, J. M., et al., 2013 Infection and immunity 81, 4592-460; Vance, D. J., et al., 2013 The Journal of biological chemistry 288, 36538-36547; Yang, Z., et al., 2014 The Journal of infectious diseases 2014 Sep. 15; 210 (6): 964-72)

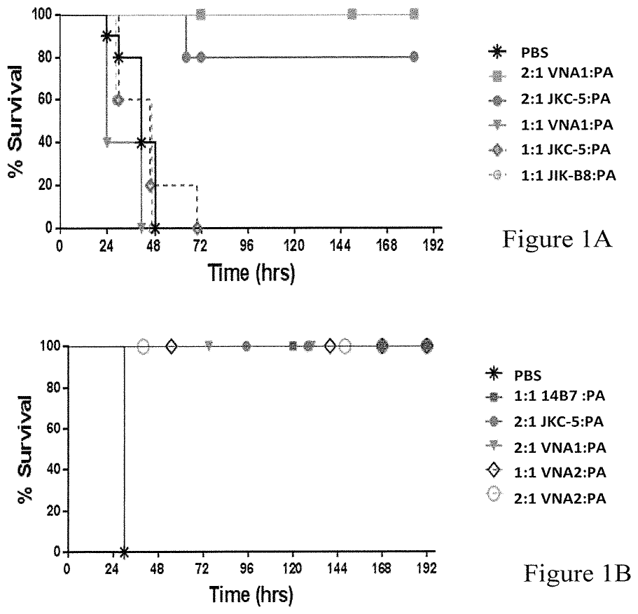

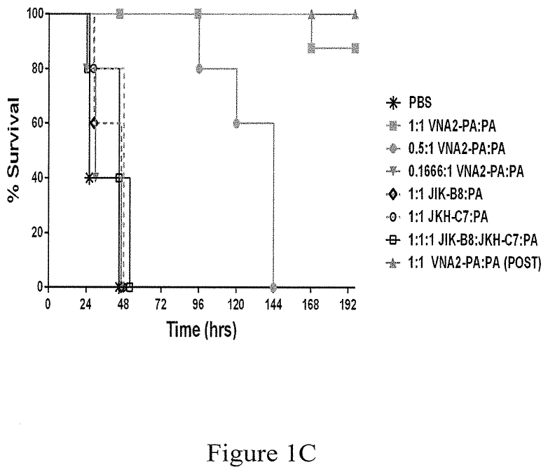

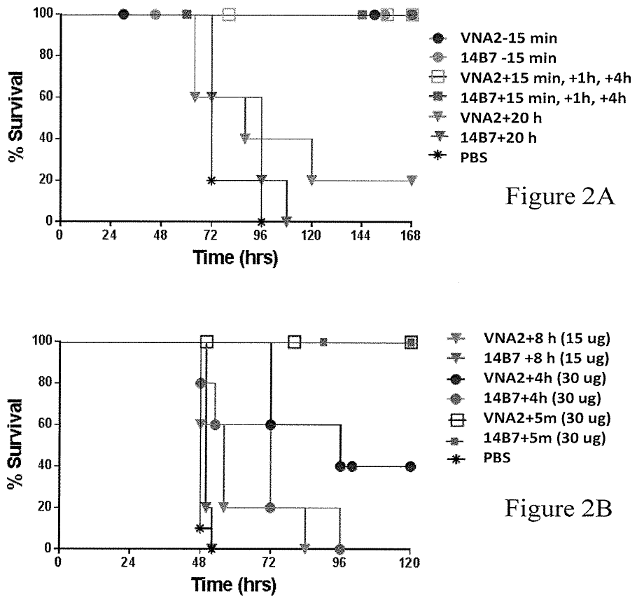

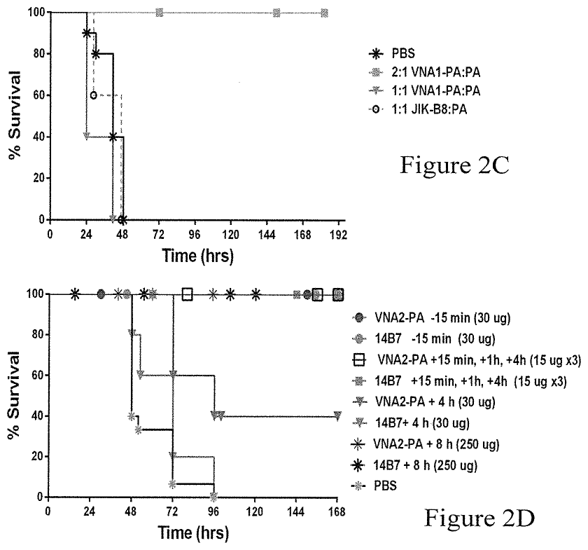

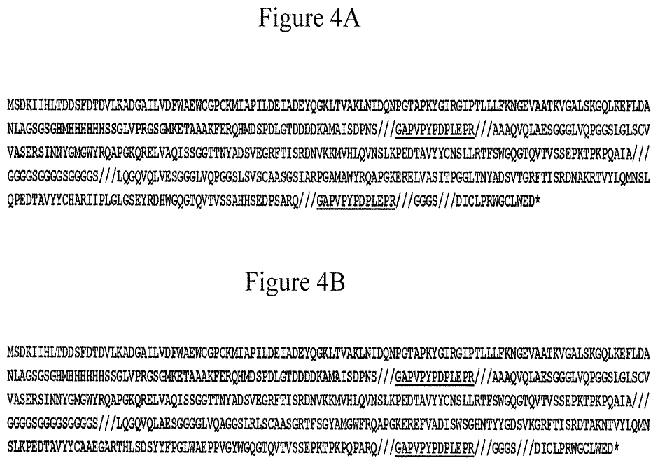

A heterodimeric VNA (VNA2-PA) was prepared to contain the two, potent neutralizing VHHs, JIK-B8 and JKH-C7, separated by a short unstructured peptide, was expressed and purified (amino acid sequence shown in FIG. 4A). This construct, VNA2-PA, was observed to have potent neutralizing toxin activity (FIG. 7C). VNA2-PA was compared to monomeric VHHs for the ability to protect mice from anthrax toxin. The toxin dose between 1-2 LD100 (45 μg LT) was administered by IV route for the Balb/cJ strain. Treatment doses were selected to test efficacy at various molar ratios of agent to toxin. Heterodimeric VNAs each bind at two separate sites on each toxin, so a dose that can fully occupy both binding sites must be present at a 2:1 molar ratio agent:toxin. Each single monomer VHH was observed as not able to protect mice or provide any beneficial effect at a 1:1 molar ratio, with percent survivals as low as that for mice administered control PBS. The heterodimeric VNA2-PA in contrast was highly protective at 1:1 (FIG. 1C) yielding 100% protection for the entire time course. Thus, VNA2-PA was able to shift the time to death significantly even at submolar ratios to toxin. Importantly, the heterodimer offered greater protection against toxin than a pool of the two VHHs used in a 1:1:1 ratio with toxin, providing evidence of the improved in vivo efficacy of the heterodimer form (FIG. 1C). VNA2-PA treatment two hour post-toxin administration was also highly protective (FIG. 1C). This finding was surprising in light of the fact that the bulk of PA has been shown to be cleaved to PA63 and removed from circulation by two hours after a bolus administration (Moayeri, M., et al., 2007 Infection and immunity 75, 5175-5184). Thus, it is here envisioned that a significant amount of active PA not measurable in circulation (plasma) may remain accessible to antibody at crucial tissue sites. A second VHH heterodimer engineered by the methods herein, VNA1-PA, incorporating as component monomers the neutralizing JIK-B8 VHH with a non-neutralizing VHH (JIJ-B8) was observed to fail to provide any protection if administered 1:1, but was fully protective in this assay if administered at a two-fold molar excess (FIG. 4B and FIG. 2C).

VNA2-PA was tested with 14B7 mAb control for protection of C57BL/6J mice against infection with a single LD100 dose of the A35 Sterne-like toxigenic B. anthracis strain. Antibody provided 15 min prior to subcutaneous spore infection or at three sequential times of dosing, at 15, 60 and 240 min post-infection, was also fully protective (FIG. 2A-FIG. 2D).

A single administration of the VNA2-PA antibody at the lower dose of 30 μg at four hours post infection resulted in survival of 2/5 mice. Mice treated with this dose of 14B7 died during the time course, likely because only one third the number of antibody molecules were present compared to VNA2-PA. Increasing the time gap between spore infection and antibody administration to eight hours resulted in a complete loss of protection unless antibody was increased to a much higher dose of 250 μg, at which dose a surprising full protection of the entire mouse group was observed (FIG. 2A-FIG. 2D).

### Example 13: Heterodimeric VHH-Based Neutralizing Agents (VNAs) Protect Against BoNT/B Toxin in Mice

BoNT/B neutralizing heterodimer VHHs were tested for the ability to protect mice from BoNT/B lethality. An amount of BoNT/B toxin of 10, 40, 100 and 500 LD50 respectively was administered by intraperitoneal injection to groups of five C57BL/6J mice. The mice receiving the toxin were treated with 2 μg of one of BoNT/B neutralizing VHH heterodimers (SEQ ID NO: 131, SEQ ID NO: 133, SEQ ID NO: 135, or SEQ ID NO: 137). Mice were monitored at least five times per day for survival and symptoms of botulism for seven days.

Mice contacted with BoNT/B toxin of 10, 40 and 100 LD50 respectively by intraperitoneal injection were treated with 2 μg of one of BoNT/B neutralizing VHH heterodimers SEQ ID NO: 131, SEQ ID NO: 133, SEQ ID NO: 135, or SEQ ID NO: 137. It was observed that the treated mice were fully protected, having a survival rate of 100%. In contrast, control mice untreated with VHH heterodimers died within 24 hours as shown in FIG. 5A-FIG. 5C.

At the even greater BoNT/B toxin concentration of 500 LD50, VHH heterodimers SEQ ID NO: 131, SEQ ID NO: 133 and SEQ ID NO: 135 provided protection for two days and VHH heterodimer SEQ ID NO: 137 showed 100% survival rate till day 7 and 80% survival rate thereafter (FIG. 5D).

### Appendices of Toxin-Binding VHH Proteins and Encoding Nucleic Acids

### Appendix B

### Anthrax Protective Antigen (PA) Positive VHHs

Included in Appendix B are the following: 8 anthrax protective antigen (PA)-binding VHHs; 16 BoNT/B-binding VHHs; and 12 BoNT/E-binding VHHs:

JKH-A4,
SEQ ID NO: 1
QVQLAETGGGLVQAGGSLRLSCSASGLTFGNYAMGWFRQAPGKEREFVASISRSGSN
TWYAEPLKGRFAISRDNDKNALYLQMNSLKPEDTAVYYCAGGSYNSDWWNYMYWGQG
TQVTVSSEPKTPKPQ
SEQ ID NO: 2
CAGGTGCAGCTGGCGGAGACGGGGGGAGGATTGGTGCAGGCTGGGGGCTCGCTGAGA
CTCTCCTGTTCAGCCTCTGGGCTCACCTTCGGGAACTATGCCATGGGCTGGTTCCGC
CAGGCTCCAGGGAAGGAGCGTGAGTTTGTAGCATCTATTTCTCGGAGTGGTAGTAAC
ACATGGTATGCAGAACCCCTGAAGGGCCGATTCGCCATCTCCAGAGACAACGACAAG
AACGCGCTCTATCTGCAAATGAACAGCCTGAAACCTGAGGACACGGCCGTTTATTAC
TGTGCTGGAGGATCTTATAATAGTGACTGGTGGAACTATATGTACTGGGGCCAGGGG
ACCCAGGTCACTGTCTCCTCAGAACCCAAGACACCAAAACCACAA
JKH-C7,
SEQ ID NO: 3
QVQLVESGGGGLVQAGGSLRLSCAASGRTFSGYAMGWFRQAPGKEREFVADISWSGH
NTYYGDSVKGRFTISRDTAKNTVYLQMNSLKPEDTAVYYCAAEGARTHLSDSYYFPG
LWAEPPVGYWGQGTQVTVSSEPKTPKPQ
SEQ ID NO: 4
CAGGTGCAGCTGGTGGAGTCGGGTGGGGGAGGACTGGTGCAGGCTGGGGGCTCTCTG
AGACTCTCCTGTGCAGCCTCTGGACGCACCTTCAGTGGCTATGCCATGGGCTGGTTC
CGCCAGGCTCCGGGGAAGGAGCGTGAGTTTGTAGCCGATATTAGCTGGAGTGGTCAT
AACACGTACTATGGAGACTCCGTGAAGGGCCGATTCACCATCTCCAGAGACACCGCC
AAGAACACGGTGTATCTGCAAATGAACAGCCTGAAACCTGAGGACACGGCCGTTTAT
TACTGTGCAGCGGAGGGGGCCCGTACACACCTTAGTGATAGTTACTACTTCCCGGGC
CTCTGGGCCGAACCCCCCGTGGGCTACTGGGGCCAGGGGACCCAGGTCACTGTCTCC
TCAGAACCCAAGACACCAAAACCACAA
JKH-D12,
SEQ ID NO: 5
QVQLVETGGGLVQAGGTLRLSCAASGRTFTSYYIGWFRQEPGKEREFVASIGWTDDN
TYYADSVKGRFTISRDNAETTAYLQMSGLKPEDTAVYYCAADYGSGIRAWYNWIYWG
QGTQVTVSSEPKTPKPQ
SEQ ID NO: 6
CAGGTGCAGCTGGTGGAGACCGGGGGAGGATTGGTGCAGGCTGGGGGCACTCTGAGA
CTCTCCTGTGCAGCCTCTGGACGTACCTTCACGAGCTATTACATTGGCTGGTTCCGC
CAGGAACCAGGGAAGGAGCGTGAGTTTGTAGCAAGTATCGGCTGGACCGATGATAAC
ACATACTATGCAGACTCCGTGAAGGGCCGATTCACCATCTCCAGAGACAACGCCGAG
ACCACGGCATATCTGCAAATGTCGGGCCTGAAACCTGAGGACACGGCCGTTTATTAC
TGTGCAGCCGACTACGGGTCAGGGATACGGGCCTGGTATAATTGGATTTACTGGGGC
CAGGGGACCCAGGTCACCGTCTCCTCAGAACCCAAGACACCAAAACCACAA
JKM-A6,
SEQ ID NO: 7
QLQLAETGGGLVQPGGSLRLSCAASGATLDTYIITWFRQAPGKEREAVSCINRSGST
TYSDSVKGRFTISRDNAQKTVYLQMNSLNPEDTAIYYCAADASYRTCGGSWWNWAYW
GQGTQVTVSSEPKTPKPQ
SEQ ID NO: 8
CAGTTGCAGCTCGCGGAGACGGGAGGAGGCTTGGTGCAGCCTGGGGGGTCTCTGAGA
CTCTCCTGTGCAGCCTCTGGCGCCACTTTGGATACTTATATCATAACCTGGTTCCGC
CAGGCCCCAGGGAAGGAGCGTGAGGCCGTCTCATGTATTAATCGTAGTGGTAGCACG
ACCTATTCAGACTCCGTGAAGGGCCGATTCACCATCTCCAGAGACAACGCCCAGAAA
ACGGTGTATCTGCAGATGAACAGCCTGAACCCTGAGGACACAGCCATTTATTACTGC
GCAGCGGATGCTTCGTACCGTACTTGCGGCGGGAGTTGGTGGAATTGGGCGTACTGG
GGCCAGGGGACCCAGGTCACCGTCTCCTCAGAACCCAAGACACCAAAACCACAA
JKO-A4,
SEQ ID NO: 9
QVQLAESGGGSVQPGGSLRLSCAASGFTFSSYTMSWVRQAPGKGIEWVSDINGGGDR
TDYADSVKGRFTISRDNARNTLYLQMNSLQPEDTAVYYCAKDLSYVSGTYFANDWGQ
GTQVTVSSEPKTPKPQ
SEQ ID NO: 10
CAGGTGCAGCTCGCGGAGTCTGGAGGAGGCTCGGTGCAACCTGGGGGGTCTCTGAGA
CTCTCCTGTGCAGCCTCTGGATTCACCTTCAGTAGTTATACTATGAGCTGGGTCCGC
CAGGCTCCAGGAAAGGGGATCGAGTGGGTCTCAGATATTAATGGGGGTGGTGATAGA
ACAGACTATGCAGACTCCGTGAAGGGCCGATTCACCATCTCCAGAGACAACGCCAGG
AACACGCTGTATCTGCAAATGAACAGCCTGCAACCTGAGGACACGGCCGTGTATTAC
TGTGCAAAAGATCTGAGCTACGTTAGTGGTACTTATTTCGCGAACGACTGGGGCCAG
GGGACCCAGGTCACCGTCTCCTCCGAACCCAAGACACCAAAACCACAA
JKO-B8,
SEQ ID NO: 11
QLQLAESGGGLVQPGGSLRLSCTASGIIFDYYSVDWYRQAPGKERELVATITGDGSP
NYADSVKGRFTISRDNAKKTVYLQMNGLKPEETAVYYCHAKRTIGTKSEYWGQGTQV
TVSSEPKTPKPQ
SEQ ID NO: 12
CAGTTGCAGCTGGCGGAGTCGGGGGGAGGCTTGGTGCAGCCTGGGGGGTCTCTGAGA
CTCTCCTGTACAGCCTCTGGAATCATCTTCGATTACTATTCCGTGGACTGGTACCGC
CAGGCTCCAGGGAAGGAGCGCGAATTGGTCGCAACTATTACGGGTGATGGTAGCCCG
AACTATGCGGACTCTGTCAAGGGCCGATTCACCATCTCCAGAGACAACGCCAAGAAG
ACGGTGTATCTGCAAATGAACGGCCTGAAACCTGAGGAAACGGCCGTCTATTACTGT
CATGCCAAAAGGACTATAGGGACCAAATCTGAGTACTGGGGCCAGGGGACCCAGGTC
ACTGTCTCCTCAGAACCCAAGACACCAAAACCACAA
JKO-E12,
SEQ ID NO: 13
QVQLAETGGGLVQAGGSLRLSCLASRMSFSRRPMAWYRQAPGKQRERVATISSFGDT
TNYTDSVEGRETISRDNAKNTMYLQMNSLKPDDTAVYYCNTLLATYAWGQGTQVTVS
SEPKTPKPQ
SEQ ID NO: 14
CAGGTGCAGCTCGCGGAGACCGGGGGAGGCTTGGTGCAGGCTGGGGGTTCTCTGAGA
CTCTCCTGTTTAGCCTCTAGAATGAGCTTTAGTAGGCGCCCCATGGCCTGGTACCGC
CAGGCTCCAGGCAAGCAGCGCGAAAGGGTCGCAACTATTAGTAGTTTCGGTGATACC
ACAAACTATACAGACTCCGTGGAGGGCCGATTCACCATCTCCAGGGACAATGCCAAG
AACACGATGTATCTGCAAATGAACAGCCTGAAACCTGACGACACGGCCGTGTATTAC
TGTAACACATTACTCGCTACGTACGCCTGGGGCCAGGGGACCCAGGTCACCGTCTCC
TCAGAACCCAAGACACCAAAACCACAA
JKO-H2,
SEQ ID NO: 15
QVQLAESGGGLVQAGGSLRLSCAASGRTFSSYVMGWFRQAPGKEREFVAAISRNGGK
TYYADSVKGRFTISRDGTENTVYLQMNSLKPEDTAVYYCAAAVAASAEFVTARSNFY
EYWGQGTQVTVSSEPKTPKPQ
SEQ ID NO: 16
CAGGTGCAGCTGGCGGAGTCGGGGGGAGGATTGGTGCAGGCTGGGGGCTCTCTGAGA
CTCTCCTGTGCAGCCTCTGGACGCACCTTCAGTAGCTATGTCATGGGCTGGTTCCGC
CAGGCTCCAGGGAAGGAGCGTGAGTTTGTGGCCGCTATTAGCCGAAATGGTGGTAAG
ACCTACTATGCAGACTCCGTGAAGGGCCGATTCACCATCTCAAGAGACGGCACCGAG
AACACGGTGTATCTGCAAATGAACAGCCTGAAACCTGAGGACACGGCCGTTTATTAC
TGCGCAGCAGCCGTAGCCGCTTCTGCCGAGTTTGTTACGGCTCGCTCGAATTTTTAT
GAATATTGGGGTCAGGGGACCCAGGTCACTGTCTCCTCAGAACCCAAGACACCAAAA
CCACAA

### New BoNT/B-Binding VHHs

JLB-B7,
SEQ ID NO: 17
QVQLVETGGGLVQAGGSLRLSCEASGSVVTIKEMGWYRQAPGKEREQERDLVAAIGIGGV
TYYATSVKGRFTISRDSAKTTLRLQMSSLRPEDTAMYYCAVITDRNTGGYPDYWGQGTQV
TVTAEPKTPKPQ
SEQ ID NO: 18
CAGGTGCAGCTGGTGGAGACGGGTGGAGGCTTGGTGCAGGCTGGGGGGTCTCTGAGACTC
TCCTGTGAAGCCTCTGGAAGCGTCGTCACCATCAAAGAGATGGGCTGGTACCGACAGGCT
CCAGGAAAGGAGCGCGAACAGGAGCGCGACTTGGTCGCAGCAATTGGCATTGGTGGTGTC
ACATACTACGCAACCTCTGTGAAGGGCCGATTCACCATCTCCAGAGACAGTGCCAAGACT
ACGCTGCGTCTGCAAATGAGCAGCCTGAGACCTGAGGACACGGCCATGTATTATTGTGCG
GTCATAACTGACAGGAACACCGGTGGTTACCCGGACTACTGGGGCCAGGGGACCCAGGTC
ACTGTTACCGCAGAACCCAAGACACCAAAACCACAA
JLI-G10,
SEQ ID NO: 19
QVQLVESGGGLVQAGGSLRLSCAASILTYDLDYYYIGWVRQAPGKEREGVSCISSTDGAT
YYADSVKGRFTISRNNAKNTVYLQMNNLKPEDTAIYYCAAAPLAGRYCPASHEYGYWGQG
TQVTVSSEPKTPKPQ
SEQ ID NO: 20
CAGGTGCAGCTGGTGGAGTCCGGTGGAGGCTTGGTGCAGGCTGGGGGGTCTCTGAGACTC
TCCTGTGCAGCCTCTATACTCACTTATGATTTGGATTATTATTACATAGGCTGGGTCCGC
CAGGCCCCAGGGAAGGAGCGTGAGGGGGTCTCATGTATTAGTAGTACTGATGGTGCCACA
TACTATGCAGACTCCGTGAAGGGCCGATTCACCATCTCCAGAAACAACGCCAAGAACACG
GTGTATCTGCAAATGAACAACCTAAAACCTGAGGACACAGCCATTTATTATTGTGCAGCA
GCCCCCCTGGCTGGGCGCTACTGTCCCGCCTCGCATGAGTATGGCTACTGGGGTCAGGGG
ACCCAGGTCACCGTCTCGTCAGAACCCAAGACACCAAAACCACAA
JLI-H11,
SEQ ID NO: 21
QVQLVESGGGLVQPGESLRLSCGASGMSLDYYAIAWYRQAPGKEREGVSCISVSGSSAQY
LDSVRGRFIISKDNTKSTAYLQMNSLKPEDTAVYYCAALADCAGYASLTFDFDSWGQGTQ
VAVSSAHHSEDPS
SEQ ID NO: 22
CAGGTGCAGCTCGTGGAGTCGGGTGGAGGCTTGGTGCAGCCTGGGGAGTCTCTGAGACTC
TCCTGTGGAGCCTCTGGAATGAGTTTGGATTACTATGCCATAGCCTGGTACCGCCAGGCC
CCAGGGAAGGAGCGTGAGGGGGTCTCATGTATTAGTGTTAGTGGCAGTAGCGCACAATAT
TTAGACTCCGTGAGGGGTCGCTTCATCATCTCCAAAGACAACACCAAGAGCACGGCGTAT
CTGCAAATGAACAGCCTGAAGCCTGAAGACACAGCCGTTTATTACTGCGCAGCCCTGGCC
GACTGTGCAGGCTATGCCAGTCTTACCTTTGACTTTGATTCTTGGGGCCAGGGGACCCAG
GTCGCCGTCTCCTCGGCGCACCACAGCGAAGACCCCTCG
JLJ-F9,
SEQ ID NO: 23
QVQLVESGGGLVQAGGSLRLSCAPSRLTLDFFAIAWFRQAPGKEREGVSCISSHDGSTYY
TDSVKGRFTISKDNAKNTVYLQMNSLKPEDTAVYYCALDHNVGTCQLTQAEYDYWGQGTQ
VTVSSAHHSEDPS
SEQ ID NO: 24
CAGGTGCAGCTGGTGGAGTCCGGTGGAGGCTTGGTGCAGGCTGGGGGGTCTCTGAGACTC
TCCTGTGCACCCTCGCGATTAACTTTGGATTTCTTTGCCATAGCCTGGTTCCGCCAGGCC
CCAGGGAAGGAGCGTGAGGGGGTCTCATGTATTAGTAGTCATGATGGTAGCACATACTAC
ACAGACTCCGTGAAGGGCCGATTCACCATCTCCAAAGACAACGCCAAGAACACGGTGTAT
CTGCAAATGAACAGCCTGAAGCCTGAGGACACAGCCGTTTATTACTGTGCCCTAGACCAT
AACGTGGGTACCTGCCAACTCACCCAAGCTGAGTATGACTACTGGGGCCAGGGGACCCAG
GTCACCGTCTCCTCGGCGCACCACAGCGAAGACCCCTCG
JLJ-G3,
SEQ ID NO: 25
QVQLVESGGGLVQSGGSLRLSCAASGSIDSLYHMGWYRQAPGKERELVARVQDGGSTAYK
DSVKGRFTISRDFSRSTMYLQMNSLKPEDTAIYYCAAKSTISTPLSWGQGTQVTVSSEPK
TPKPQ
SEQ ID NO: 26
CAGGTGCAGCTGGTGGAGTCCGGGGGAGGCTTGGTGCAGTCTGGGGGGTCTCTGAGACTC
TCCTGTGCAGCCTCTGGAAGTATCGATAGTCTCTATCATATGGGCTGGTACCGCCAGGCT
CCAGGGAAGGAGCGCGAGTTGGTCGCACGAGTTCAAGATGGGGGTAGCACAGCGTACAAA
GACTCTGTGAAGGGGCGATTCACCATCTCCAGAGACTTTTCCAGGAGCACGATGTATCTG
CAAATGAACAGCCTGAAACCTGAGGACACGGCCATCTATTACTGTGCGGCGAAGAGTACA
ATTAGCACCCCCTTGTCCTGGGGCCAGGGGACCCAGGTCACCGTCTCCTCGGAACCCAAG
ACACCAAAACCACAA
JLK-D7,
SEQ ID NO: 27
QVQLVESGGGLVQAGGSLRLSCAASGFTLGHNQVAWFRQAPGKEREGVACISATGASTHY
ADPVKGRFTVSRDNTKNVVYLQVNSLKPEDTANYVCASRFSLMSIDASMCLSAPQYDRWG
QGTQVRISSEPKTPKPQ
SEQ ID NO: 28
CAGGTGCAGCTGGTGGAGTCCGGTGGAGGCTTGGTGCAGGCTGGGGGGTCTCTGAGACTC
TCCTGTGCAGCCTCTGGATTCACTTTGGGACATAATCAAGTAGCCTGGTTCCGCCAGGCC
CCAGGCAAGGAGCGTGAGGGGGTCGCGTGTATTAGCGCCACCGGTGCTAGCACACACTAT
GCAGACCCCGTGAAGGGCCGATTTACCGTCTCCAGAGACAACACCAAGAACGTGGTGTAT
CTGCAAGTGAACAGCCTGAAACCTGAGGACACGGCCAATTATGTCTGTGCAAGCAGATTC
TCCCTTATGTCGATCGATGCGAGCATGTGCCTTTCGGCGCCTCAGTATGACCGCTGGGGC
CAGGGGACCCAGGTCAGAATCTCCTCAGAACCCAAGACACCAAAACCACAA
JLK-F7,
SEQ ID NO: 29
QVQLVETGGLVQPGGSLRLSCTASGFTLGHHRVGWFRQAPGKEREGVACISATGLSSHYS
DEVIGRFTVSRDNDNNVVYLQVNGLKPEDTAVYYCASRFSLNSVDANMCLSEPQYDNWGQ
GTPVRISSEPKTPKPQ
SEQ ID NO: 30
CAGGTGCAGCTGGTGGAGACGGGTGGCTTGGTGCAGCCTGGGGGGTCTCTGAGACTCTCC
TGTACAGCCTCTGGATTCACTTTGGGACACCATCGCGTTGGCTGGTTCCGCCAGGCCCCA
GGAAAGGAGCGTGAGGGGGTCGCGTGTATTAGCGCCACTGGTCTTAGTTCACACTATTCA
GACTTCGTGATCGGCCGATTTACCGTCTCCAGAGACAACGACAACAACGTGGTGTATCTA
CAAGTGAACGGCCTGAAACCTGAGGACACAGCCGTTTATTACTGTGCAAGCAGATTCTCC
CTTAATTCGGTCGATGCGAATATGTGCCTTTCGGAGCCTCAGTATGACAACTGGGGCCAG
GGGACCCCGGTCAGAATCTCCTCAGAACCCAAGACACCAAAACCACAA
JLK-G12,
SEQ ID NO: 31
QVQLVESGGGLVQAGGSLRLSCAASEFRAEHFAVGWFRQAPGKEREGVSCVDASGDSTAY
ADSVKGRFTISRDNNKNVVYLQMDSLEPEDTGDYYCGASYFTVCAKSMRKIEYRYWGQGT
QVTVSSEPKTPKPQ
SEQ ID NO: 32
CAGGTGCAGCTGGTGGAGTCCGGTGGAGGCTTGGTGCAGGCTGGGGGGTCTCTGAGACTC
TCCTGTGCAGCCTCTGAATTCCGTGCGGAGCATTTTGCCGTGGGCTGGTTCCGCCAGGCC
CCAGGGAAGGAGCGTGAGGGGGTCTCATGTGTAGACGCGAGTGGTGATAGTACAGCATAT
GCGGACTCTGTGAAGGGCCGATTCACCATCTCCAGAGACAACAACAAGAACGTAGTGTAT
CTGCAAATGGACAGCCTGGAACCTGAAGACACAGGAGATTATTATTGTGGAGCCTCGTAC
TTTACTGTCTGCGCCAAGAGCATGCGGAAAATTGAATATAGGTACTGGGGCCAGGGGACC
CAGGTCACCGTCTCCTCAGAACCCAAGACACCAAAACCACAA
JLO-C8,
SEQ ID NO: 33
QVQLAESGGGLVQPGGSLRLSCAASGRALNYYVIGWFRQAPGKEREGVSCIASSEAYTDY
ADSVQGRFTISRDKALNTVYLDMKRLKPDDTAVYYCAARLRDPNWCGRNADEYDSWGQGT
QVTVSSEPKTPKPQ
SEQ ID NO: 34
CAGGTGCAGCTCGCGGAGTCAGGCGGAGGCTTGGTGCAGCCTGGGGGGTCTCTGAGACTC
TCCTGTGCAGCCTCTGGACGCGCTTTGAATTATTATGTCATAGGCTGGTTCCGCCAGGCC
CCAGGGAAGGAGCGTGAGGGGGTCTCATGTATTGCGAGTAGCGAAGCCTACACAGACTAT
GCAGACTCCGTGCAAGGCCGATTCACCATCTCGAGAGACAAGGCTCTGAATACGGTGTAT
TTGGATATGAAGCGCCTGAAACCTGACGACACAGCCGTTTATTATTGTGCAGCCCGGTTG
CGTGATCCTAATTGGTGCGGGCGGAATGCGGATGAGTATGACTCCTGGGGCCAGGGGACC
CAGGTCACCGTCTCCTCAGAACCCAAGACACCAAAACCACAA
JLO-G7,
SEQ ID NO: 35
QVQLVESGGGLVQAGGSLRLSCAASGFPFGSYYMSWVRQAPGKGPEWVSDISNGGIITRY
SDSVKGRFTISRDNAKNILYLQMNSLKPEDTALYFCATGTGRDWSREYRGQGTQVTVSSE
PKTPKPQ
SEQ ID NO: 36
CAGGTGCAGCTGGTGGAGTCCGGTGGAGGCTTGGTGCAGGCTGGGGGGTCTCTGAGACTC
TCCTGTGCAGCCTCTGGATTCCCCTTCGGTAGTTACTACATGAGCTGGGTCCGCCAGGCT
CCAGGAAAGGGGCCCGAGTGGGTCTCAGATATTAGCAATGGTGGTATTATTACAAGGTAT
TCAGACTCCGTGAAGGGCCGATTCACCATCTCCCGAGACAACGCCAAGAACATATTGTAT
CTGCAAATGAACAGCCTGAAACCTGAAGACACGGCCCTGTATTTCTGTGCGACAGGGACC
GGTAGAGACTGGAGCAGGGAGTACCGGGGCCAGGGGACCCAGGTCACCGTCTCCTCAGAA
CCCAAGACACCAAAACCACAA
JLO-G11,
SEQ ID NO: 37
QVQLAESGGGLVQPGGSLRLSCEASGFHLEHFAVGWFRQAPGKEREGVSCISASGDSTTY
ADSVKGRSTISKDNAKNAVYLQMDSLRPEDTGDYYCAASHFSVCGKNIRKIEYRYWGQGT
PVTVSSEPKTPKPQ
SEQ ID NO: 38
CAGGTGCAGCTCGCGGAGTCTGGTGGAGGCTTGGTGCAGCCTGGGGGGTCTCTGAGACTC
TCCTGTGAAGCCTCAGGATTCCATTTGGAGCATTTTGCCGTAGGCTGGTTCCGCCAGGCC
CCAGGGAAGGAGCGTGAGGGGGTCTCATGTATAAGCGCGAGTGGTGATAGTACAACGTAT
GCAGACTCCGTGAAGGGCCGATCCACCATCTCCAAAGACAACGCCAAGAACGCGGTGTAT
CTGCAAATGGACAGCCTGAGACCCGAGGACACAGGCGATTATTACTGTGCAGCCTCGCAC
TTCAGTGTCTGCGGCAAGAACATTCGGAAAATTGAGTATAGGTACTGGGGCCAGGGGACC
CCGGTCACCGTCTCCTCAGAACCCAAGACACCAAAACCACAA
JLU-A4,
SEQ ID NO: 39
QVQLVETGGGLVQPGGSLRLSCVVSGLTENSNYMSWVRQAPGKGPELVSYINSEDGSTFY
ADSVKGRFTISRDNNENTLYLQMSSLKPEDTARYYCALGIAGATRGQGTQVTVSSEPKTP
KPQ
SEQ ID NO: 40
CAGGTGCAGCTCGTGGAGACCGGGGGAGGCTTGGTGCAGCCGGGGGGGTCTCTGAGACTC
TCCTGTGTAGTGTCTGGATTAACCTTCAATAGCAACTACATGAGTTGGGTCCGCCAGGCT
CCAGGGAAGGGGCCCGAGTTGGTCTCATATATTAATTCTGAAGATGGTAGTACCTTTTAT
GCAGACTCCGTGAAGGGCCGATTCACCATCTCGCGAGACAACAACGAGAATACACTGTAT
CTGCAAATGAGCAGCCTGAAGCCTGAGGACACGGCCCGCTATTACTGTGCACTGGGGATC
GCTGGTGCAACTCGGGGCCAGGGGACCCAGGTCACCGTCTCCTCAGAACCCAAGACACCA
AAACCACAA
JLU-D10,
SEQ ID NO: 41
QVQLVESGGGLVQPGGSLRLSCAASGFTLDSYAIGWFRQAPGKEREGVACISASGSGTDY
VDSVKGRFTVSRDQAKSMVFLQMNNMKPEDAAVYYCAADYRPRPLPIQAPCTMTGGNYWG
QGTQVTVSSEPKTPKPQ
SEQ ID NO: 42
CAGGTGCAGCTCGTGGAGTCAGGGGGAGGCTTGGTGCAGCCTGGGGGGTCTCTGAGACTC
TCCTGTGCAGCCTCTGGATTCACTTTAGATAGTTATGCAATAGGCTGGTTCCGCCAGGCC
CCAGGGAAGGAGCGTGAGGGGGTCGCATGTATTAGTGCTAGTGGTAGTGGCACGGACTAT
GTAGACTCCGTGAAGGGCCGATTCACCGTCTCCAGAGACCAGGCCAAGAGCATGGTGTTT
CTGCAAATGAACAACATGAAACCTGAGGACGCAGCCGTTTATTACTGTGCAGCAGATTAT
CGGCCGAGGCCCCTGCCGATTCAGGCGCCGTGTACAATGACAGGTGGCAACTACTGGGGC
CAGGGGACCCAGGTCACCGTCTCCTCAGAACCCAAGACACCAAAACCACAA
JLU-H6,
SEQ ID NO: 43
QVQLVESGGGLVQPGGSLTLSCVASGSNLDYFAIGWFRQAPGKEREGVSCISTSSDMSKY
ADSVKGRFTISRDNTRNTVYLQMNSLEPEDTAVYYCAAKRRRYGLDRDMCLMDSVGMDVW
GKGTLVTVSSAHHSEDPS
SEQ ID NO: 44
CAGGTGCAGCTGGTGGAGTCGGGGGGAGGCTTGGTGCAGCCTGGGGGGTCTCTGACACTC
TCCTGTGTAGCCTCTGGATCCAATTTGGATTATTTTGCGATAGGCTGGTTCCGCCAGGCC
CCAGGGAAGGAGCGTGAGGGGGTCTCATGTATTAGTACGAGTAGTGACATGTCAAAGTAT
GCAGACTCCGTGAAGGGCCGCTTCACCATCTCCAGAGACAACACCAGGAACACGGTGTAT
CTGCAAATGAACAGCCTGGAACCCGAAGATACGGCCGTTTATTATTGTGCAGCAAAGCGC
CGCCGATATGGTCTCGATCGTGATATGTGTCTTATGGATTCGGTCGGCATGGACGTGTGG
GGCAAAGGGACCCTGGTCACCGTCTCCTCGGCGCACCACAGCGAAGACCCCTCG
JLU-H9,
SEQ ID NO: 45
QVQLVESGGGLVQPGGSLRLSCAAPGFTLDYYAIGWFRQAPGKEREGVSCIRSRGDRTNY
ADSVKGRFTVSRDNAKNTAYLQMNNLKPEDTGVYFCAAAPRTTVQDLCVTPLLGGADWVS
WGQGTQVTVSSEPKTPKPQ
SEQ ID NO: 46
CAGGTGCAGCTCGTGGAGTCAGGAGGAGGCTTGGTGCAGCCTGGGGGGTCTCTGAGACTC
TCCTGTGCAGCTCCTGGATTCACTTTGGATTATTATGCCATAGGCTGGTTCCGCCAGGCC
CCAGGGAAGGAGCGTGAGGGGGTCTCATGTATTCGTAGTCGTGGTGATCGGACAAATTAT
GCAGACTCCGTGAAGGGCCGATTCACCGTCTCCAGAGACAACGCCAAGAACACGGCGTAT
CTGCAAATGAACAACCTGAAACCTGAGGACACAGGCGTTTATTTCTGTGCAGCTGCTCCG
AGGACTACTGTTCAGGATTTGTGTGTAACCCCTCTTTTGGGGGGTGCTGACTGGGTTTCC
TGGGGCCAGGGGACCCAGGTCACCGTCTCCTCGGAACCCAAGACACCAAAACCACAA
JLU-H10,
SEQ ID NO: 47
QLQLVESGGGLVQPGGSLRLSCAASGFPLGDYTVGWFRQAPGKEREGVSCISKGSRGLRY
GDSVKGRFTVARDNAKSTVTLQMDSLKPEDTAVYSCAAGPAMENQCHMVDNYFTYWGQGT
QVTVSSAHHSEDPS
SEQ ID NO: 48
CAGTTGCAGCTGGTGGAGTCTGGCGGAGGCTTGGTGCAGCCTGGGGGGTCTCTGAGACTC
TCCTGTGCAGCCTCTGGATTCCCTTTGGGTGATTATACCGTGGGCTGGTTCCGCCAGGCC
CCAGGGAAGGAGCGTGAGGGGGTCTCATGTATTAGTAAAGGTAGTAGAGGCTTAAGATAC
GGAGACTCCGTGAAAGGCCGATTCACCGTTGCCAGAGACAACGCCAAGAGCACGGTAACT
CTGCAAATGGACAGCCTGAAACCGGAGGACACAGCCGTTTATTCTTGTGCTGCAGGGCCG
GCCATGTTCAATCAATGTCATATGGTCGACAATTACTTTACATACTGGGGTCAGGGGACC
CAGGTCACCGTCTCCTCGGCGCACCACAGCGAAGACCCCTCG

### New BoNT/E-Binding VHHs

JLD-B12,
SEQ ID NO: 49
QVQLVETGGGLVQAGGSLRLSCAASGRTFSNYAMGWFRQAPGKEREFVAAISWSGAHTYY
ADSVKGRFTISRDNAKSTMYLQMNSLKPEDTAVYYCNADLERYSDFGREVDDYWGQGTQV
TVSSEPKTPKPQ
SEQ ID NO: 50
CAGGTGCAGCTCGTGGAGACAGGTGGAGGATTGGTGCAGGCTGGGGGGTCTCTGAGACTC
TCCTGTGCAGCCTCTGGACGCACCTTCAGTAACTATGCCATGGGCTGGTTCCGCCAGGCT
CCAGGGAAGGAGCGTGAGTTTGTCGCAGCTATTAGCTGGAGTGGTGCTCACACATACTAT
GCAGACTCCGTGAAGGGCCGATTCACCATCTCCAGAGACAACGCCAAGAGCACGATGTAT
CTGCAAATGAACAGCCTGAAACCTGAGGACACGGCCGTCTATTACTGTAATGCAGATCTC
GAGCGGTATAGTGACTTCGGTAGGGAGGTGGATGACTACTGGGGCCAGGGGACCCAGGTC
ACCGTCTCCTCAGAACCCAAGACACCAAAACCACAA
JLE-A12,
SEQ ID NO: 51
QVQLVESGGGLVQPGGSLRLSCTASGLTLAKWTINWFRQAPGKEREGISCISSSSGSTYY
ADSVKGRFTISRDNAENTVYLQMS SLKPEDTAVYYCAADS FKGCTFLSSTTHYNNMDYWG
KGTLVTVSSAHHSEDPS
SEQ ID NO: 52
CAGGTGCAGCTCGTGGAGTCGGGTGGAGGCTTGGTGCAGCCTGGGGGGTCTCTGAGACTC
TCGTGTACAGCCTCTGGATTAACTTTGGCTAAGTGGACCATCAACTGGTTCCGCCAGGCC
CCAGGGAAGGAGCGCGAGGGGATCTCATGTATTAGTAGCAGTAGTGGTAGCACATACTAT
GCAGACTCCGTGAAGGGCCGATTCACCATCTCCAGAGACAACGCCGAAAACACGGTATAT
CTGCAAATGAGCAGCCTGAAACCTGAGGACACGGCCGTTTATTACTGTGCAGCGGATTCT
TTTAAGGGCTGTACGTTCCTCAGTAGTACTACCCATTACAACAACATGGACTACTGGGGC
AAAGGGACCCTGGTCACCGTCTCCTCAGCGCACCACAGCGAAGACCCCTCG
JLE-B10,
SEQ ID NO: 53
QVQLVESGGGLVQSGGSLRLSCAASRRTASNYAVAWFRQAPGKEREFVAAIGWSDDVTYY
ADSVKGRFTVSRDNAKNTVYLQMNGLEPEDTAVYYCTTNGDRYSYRTASSYHYWGQGTQV
TVSSAHHSEDPS
SEQ ID NO: 54
CAGGTGCAGCTCGTGGAGTCGGGTGGGGGATTGGTGCAGTCTGGGGGCTCTCTGAGACTC
TCCTGTGCAGCCTCTAGACGCACCGCCAGTAACTATGCCGTGGCCTGGTTCCGCCAGGCT
CCAGGAAAGGAGCGTGAGTTTGTAGCAGCGATTGGCTGGAGTGATGATGTCACGTATTAC
GCAGACTCCGTGAAGGGCCGATTCACCGTCTCCAGAGACAACGCCAAGAACACGGTGTAT
CTGCAAATGAACGGCCTGGAACCTGAGGACACGGCCGTTTATTACTGTACAACAAATGGT
GATAGATACAGTTACAGGACGGCATCCAGCTATCACTACTGGGGCCAGGGGACCCAGGTC
ACCGTCTCCTCAGCGCACCACAGCGAAGACCCCTCG
JLE-C7,
SEQ ID NO: 55
QVQLAETGGGSVQTGGSLRLSCAASGLPFRNYAMAWERQAPGKEREFVAAISREGGRTYY
ADFVKGRFTISRDNGRNTIYLEMNSLASEDTAIYYCAGVEGAYTYRTGASYTYWGQGTQV
TVSSEPKTPKPQ
SEQ ID NO: 56
CAGGTGCAGCTCGCGGAGACTGGGGGAGGATCGGTGCAGACTGGGGGCTCTCTGAGGCTC
TCCTGTGCAGCCTCTGGACTGCCCTTCAGAAACTATGCCATGGCCTGGTTCCGCCAGGCT
CCAGGGAAGGAGCGTGAGTTTGTAGCAGCTATTAGTCGGGAAGGCGGGAGGACATACTAT
GCAGACTTCGTGAAGGGCCGATTCACCATCTCCAGAGACAACGGCAGGAACACGATATAT
CTGGAGATGAACAGCCTGGCATCGGAGGATACGGCCATTTATTACTGTGCCGGTGTCGAG
GGTGCTTATACTTATCGTACCGGGGCCTCGTATACTTACTGGGGCCAGGGGACCCAGGTC
ACCGTCTCCTCAGAACCCAAGACACCAAAACCACAA
JLE-E5,
SEQ ID NO: 57
QVQLVETGGGLVQAGGSLRLSCAASGRSYAMGWFRQGPGKEREFVATISWSSTNTWYADS
VKGRFTISRDNAKNTVYLQMNSLKPEDTAVYYCAASHRESDYPMRSEDGMDYWGKGTLVT
VSSEPKTPKPQ
SEQ ID NO: 58
CAGGTGCAGCTGGTGGAGACGGGGGGAGGATTGGTGCAGGCTGGGGGCTCTCTGAGACTC
TCCTGTGCAGCCTCTGGACGCAGTTATGCCATGGGCTGGTTCCGCCAGGGTCCAGGGAAG
GAGCGTGAGTTTGTAGCCACTATCAGTTGGAGTAGTACTAACACATGGTATGCAGATTCC
GTGAAGGGCCGATTCACCATCTCTAGAGACAACGCCAAGAACACGGTGTATCTGCAAATG
AACAGCCTGAAACCTGAGGACACGGCTGTTTATTACTGTGCAGCGAGCCATCGTTTTAGC
GACTATCCCATGAGGTCAGAGGACGGCATGGACTACTGGGGCAAAGGGACCCTGGTCACC
GTCTCCTCAGAACCCAAGACACCAAAACCACAA
JLE-E9,
SEQ ID NO: 59
QVQLVETGGGLVQAGGSLRLSCAASGRTFSSYSMGWFRQAPGKEREYVAAVNSNGDSTFY
ADSIKGRFTVSRDAAKNTVYLQMNSLKPEDTALYYCAAVYGRYTYQSPKSYEYWGQGTQV
TVSSEPKTPKPQ
SEQ ID NO: 60
CAGGTGCAGCTGGTGGAGACGGGAGGAGGATTGGTGCAGGCTGGGGGCTCTCTGAGACTC
TCGTGTGCAGCCTCTGGACGCACCTTCAGTAGCTATTCCATGGGCTGGTTCCGCCAGGCT
CCAGGGAAGGAGCGTGAGTATGTAGCAGCAGTTAACTCCAATGGCGACAGTACATTCTAT
GCCGACTCCATTAAGGGCCGATTCACCGTCTCCAGAGACGCCGCCAAGAACACAGTCTAT
CTGCAAATGAACAGCCTGAAACCTGAGGACACGGCCCTTTATTACTGTGCAGCTGTCTAC
GGTAGATACACTTACCAGTCCCCAAAATCGTATGAGTACTGGGGCCAGGGGACCCAGGTC
ACCGTCTCCTCAGAACCCAAGACACCAAAACCACAA
JLE-G6,
SEQ ID NO: 61
QLQLVETGGGLVKPGGSLRLSCVVSGFTEDDYRMAWVRQAPGKELEWVSSIDSWSINTYY
EDSVKGRFTISTDNAKNTLYLQMSSLKPEDTAVYYCAAEDRLGVPTINAHPSKYDYNYWG
QGTQVTVSSEPKTPKPQ
SEQ ID NO: 62
CAGTTGCAGCTCGTGGAGACTGGTGGAGGCTTGGTGAAGCCTGGGGGTTCTCTGAGACTC
TCCTGTGTAGTCTCCGGATTCACTTTTGATGATTATCGCATGGCTTGGGTCCGCCAGGCT
CCAGGGAAGGAGCTGGAGTGGGTGTCCAGTATAGATAGTTGGAGTATCAACACATACTAT
GAAGACTCCGTGAAGGGCCGGTTCACCATCTCCACAGACAACGCCAAGAATACACTGTAT
CTGCAAATGAGCAGCCTGAAACCTGAGGACACGGCCGTGTATTACTGTGCAGCAGAGGAC
CGCTTAGGTGTACCGACTATTAACGCCCACCCTTCAAAATATGATTATAACTACTGGGGG
CAGGGGACCCAGGTCACCGTCTCCTCAGAACCCAAGACACCAAAACCACAA
JLE-H5,
SEQ ID NO: 63
QVQLVESGGGLVQAGGSLRLSCAASGRTFTSYAMGWFRQAPGKEREFVASISWRGSYTYY
SDSVKGRFTISRDYAENTMYLQMNSLKPEXTGRYYCATLTGDVSVGEYDNRGQGTQVTVS
SAHHSEDPS
SEQ ID NO: 64
CAGGTGCAGCTGGTGGAGTCCGGTGGAGGCTTGGTGCAGGCTGGGGGGTCTCTGAGACTC
TCCTGTGCAGCCTCTGGACGCACCTTCACTAGTTATGCCATGGGCTGGTTCCGCCAGGCT
CCAGGGAAGGAGCGTGAGTTTGTAGCGTCTATTAGCTGGCGCGGTAGTTACACATACTAT
TCAGACTCCGTGAAGGGCCGATTCACCATCTCCAGAGATTACGCCGAGAACACGATGTAT
CTGCAAATGAACAGCCTGAAACCTGAGGNNACGGGCAGATATTACTGTGCAACCTTAACC
GGCGACGTGAGTGTCGGCGAGTATGACAACCGGGGCCAGGGGACCCAGGTCACTGTCTCC
TCAGCGCACCACAGCGAAGACCCCTCG
JLF-H5,
SEQ ID NO: 65
QVQLVESGGGSVQPGGSLRLSCVASGFTFTNYAMAWVRQVSGKGLEGVAAISSEGFIYIP
DSVKGRFTISRDNAKNTVYLQMDNLQSEDTAIYHCAAVDWKRVAAMNSYNMDYWGKGTPV
TVSAEPKTPKPQ
SEQ ID NO: 66
CAGGTGCAGCTGGTGGAGTCGGGGGGAGGCTCGGTGCAGCCTGGGGGGTCTCTGAGACTC
TCCTGTGTAGCCTCTGGATTCACCTTCACTAATTACGCGATGGCCTGGGTCCGCCAGGTA
TCAGGGAAGGGGCTCGAGGGTGTGGCCGCTATTAGTAGTGAGGGTTTCATATATATCCCA
GACTCAGTGAAGGGCCGATTCACCATCTCCAGAGACAACGCCAAGAACACGGTGTATCTA
CAAATGGACAACCTCCAGTCTGAGGATACGGCCATATATCACTGTGCGGCAGTTGATTGG
AAACGGGTCGCCGCGATGAACAGCTACAACATGGACTACTGGGGAAAAGGGACCCCGGTC
ACCGTCTCCGCAGAACCCAAGACACCAAAACCACAA
JLG-G8,
SEQ ID NO: 67
QLQLVESGGGLVQAGGSLRLSCAASGRTESSYAMGWFRQAPGKEREHVAAISWSGGYTYY
ANSVKGRFTISRDNAKNTVYLQMNSLKPEDTAVYYCNGVQDHSDSLQNWGQGTQVTVSSE
PKTPKPQ
SEQ ID NO: 68
CAGTTGCAGCTGGTGGAGTCGGGCGGAGGATTGGTGCAGGCTGGGGGGTCTCTGAGACTC
TCCTGTGCAGCCTCTGGACGCACCTTCAGTAGCTATGCCATGGGCTGGTTCCGCCAGGCT
CCAGGGAAGGAACGTGAGCATGTCGCAGCTATTAGCTGGAGTGGTGGTTACACATACTAT
GCAAACTCCGTGAAGGGCCGATTCACCATCTCCAGAGACAACGCCAAGAACACGGTGTAT
CTGCAAATGAACAGCCTGAAACCTGAGGACACGGCCGTCTATTACTGTAATGGAGTTCAG
GACCATAGCGACTCCCTTCAGAACTGGGGCCAGGGGACCCAGGTCACCGTCTCCTCAGAA
CCCAAGACACCAAAACCACAA
JLG-G12,
SEQ ID NO: 69
QLQLVETGGGLVQAGGSLRLSCAASGRTESSYAVGWFRQAPGKEREFVAAISWSGSYAYY
ADSVKGRFTISRDNAKNTVYLQMNSLKPEDTAVYYCNGDLEGYSNHETGDYWGQGTQVTV
SSEPKTPKPQ
SEQ ID NO: 70
CAGTTGCAGCTGGTGGAGACGGGAGGAGGATTGGTGCAGGCTGGGGGGTCTCTGAGACTC
TCCTGTGCAGCCTCTGGACGCACCTTCAGTAGTTATGCCGTGGGCTGGTTCCGCCAGGCT
CCAGGGAAGGAGCGTGAGTTTGTCGCAGCTATTAGCTGGAGTGGTAGTTACGCATACTAT
GCAGACTCCGTGAAGGGCCGATTCACCATCTCCAGAGACAACGCCAAGAACACGGTGTAT
CTGCAAATGAACAGCCTGAAACCTGAGGACACGGCCGTCTATTACTGTAATGGAGATCTT
GAGGGTTATAGCAACCATGAAACCGGGGACTACTGGGGCCAGGGGACCCAGGTCACCGTC
TCCTCAGAACCCAAGACACCAAAACCACAA
JLH-H4,
SEQ ID NO: 71
QLQLAESGGGLVQAGGSLRLSCAASGRTFSSYAMGWFRQAPGKEREFVAAISWIGGYTYY
ASSVKGRFTISRDNAKNTMYLQMNSLKPEDTAVYYCNADLESYSEYPESYYWGQGTQVTV
SSEPKTPKPQ
SEQ ID NO: 72
CAGTTGCAGCTGGCGGAGTCGGGAGGAGGATTGGTGCAGGCTGGGGGGTCTCTGAGACTC
TCCTGTGCAGCCTCTGGACGCACCTTCAGTAGCTATGCCATGGGCTGGTTCCGCCAGGCT
CCAGGGAAGGAGCGTGAGTTTGTCGCAGCTATTAGCTGGACTGGTGGTTACACATACTAT
GCAAGCTCCGTGAAGGGCCGATTCACCATCTCCAGAGACAATGCCAAAAACACGATGTAT
CTGCAAATGAACAGCCTGAAACCGGAGGACACGGCCGTCTATTACTGTAATGCAGATTTA
GAATCCTATAGCGAGTATCCCGAGAGCTACTACTGGGGCCAGGGGACCCAGGTCACCGTC
TCCTCAGAACCCAAGACACCAAAACCACAA

### Appendix C

Included in Appendix C are the following: Amino acid and nucleic acid sequences of 2 anthrax edema factor (EF)-binding VHHs; 7 anthrax lethal factor (LF)-binding VHHs; and 6 VHHs binding both anthrax EF and LF (EF/LF cross-specific)

### New Anthrax EF-Binding VHHs

JMN-E2,
SEQ ID NO: 73
QVQLAESGGGLVQAGGSLTLSCAASGLNFDKYAIGWYRQAPGKEREGVSCISKYYNH
RMYSDSVKGRFTVSSNYAKNTVYLQMTNLKPEDTAVYYCAAGCIDPEDWGQGTQVTV
SSEPKTPKPQ
SEQ ID NO: 74
CAGGTGCAGCTGGCGGAGTCGGGGGGAGGCTTGGTGCAGGCTGGGGGGTCTCTGACA
CTCTCCTGTGCAGCCTCTGGCCTCAATTTCGATAAATATGCCATAGGCTGGTACCGC
CAGGCCCCAGGGAAAGAGCGTGAGGGGGTTTCATGTATTAGTAAGTATTACAATCAT
CGGATGTATAGTGACTCCGTGAAGGGCCGATTCACCGTCTCCAGTAACTATGCCAAG
AACACGGTGTACCTGCAAATGACCAATCTGAAACCGGAGGATACGGCCGTTTATTAC
TGTGCGGCAGGGTGTATTGACCCGGAAGATTGGGGCCAGGGGACCCAGGTCACCGTC
TCCTCAGAACCCAAGACACCAAAACCACAA
JMN-F3,
SEQ ID NO: 75
QVQLVETGGGQVQTGGSLRLSCAASEPTFTPKVVGWFRQAPVKERDEVATITIRTGR
TLYADSVKGRFTISGDGANNTVYLQMNGLKPEDTAVYYCAASLPLAIPPTQASAYEY
WGLGTQVTVSSEPKTPKPQ
SEQ ID NO: 76
CAGGTGCAGCTGGTGGAGACCGGGGGAGGCCAGGTGCAGACTGGGGGATCTCTGAGA
CTCTCTTGCGCAGCCTCTGAACCCACCTTCACTCCGAAAGTTGTGGGCTGGTTCCGC
CAGGCTCCAGTGAAGGAGCGTGACTTTGTAGCAACTATAACAATCCGTACCGGTCGC
ACACTCTATGCAGATTCCGTGAAGGGCCGATTCACCATCTCCGGAGACGGCGCCAAC
AATACGGTGTATCTACAAATGAACGGCCTGAAACCTGAGGACACGGCCGTTTATTAC
TGCGCCGCATCTCTTCCGCTAGCAATACCACCGACGCAGGCTTCGGCATATGAATAC
TGGGGCCTGGGGACCCAGGTCACCGTCTCCTCAGAACCCAAGACACCAAAACCACAA

### New Anthrax LF-Binding VHHs

JMO-A2,
SEQ ID NO: 77
QVQLVETGGGLVQPGGSLRLSCSVSGLHERFANMGWFRQAPGKQRELVAYITTGDNT
NYVDHVKGRFTISRDNAKNTVYLQMNSLKPEDTAVYYCNIVNALGEENPRNDWGQGT
QVTVSSEPKTPKPQ
SEQ ID NO: 78
CAGGTGCAGCTGGTGGAGACGGGGGGAGGCTTGGTGCAGCCTGGGGGGTCTCTGAGA
CTCTCCTGTTCAGTCTCTGGCCTCCACTTCAGGTTCGCGAACATGGGATGGTTTCGC
CAGGCTCCAGGGAAGCAGCGCGAGTTGGTCGCATATATTACTACTGGTGATAACACT
AACTATGTAGACCACGTGAAGGGCCGATTCACCATCTCCAGAGACAACGCCAAGAAC
ACGGTGTATCTGCAAATGAACAGCCTGAAACCTGAAGACACGGCCGTCTACTACTGT
AATATAGTCAATGCGCTGGGGGAGTTCAATCCCCGAAACGACTGGGGCCAGGGGACC
CAGGTCACCGTCTCCTCAGAACCCAAGACACCAAAACCACAA
JMO-B3,
SEQ ID NO: 79
QVQLVETGGGWVQAGGSLRLSCAASGRAASGNAMAWFRQAPGKEREFVALISWSGGR
PYYANSVKGRFAISRDNATNTVYLQMNRLKPEDTAVYYCAASPTIAILPTPYDYWGQ
GTQVTVSSEPKTPKPQ
SEQ ID NO: 80
CAGGTGCAGCTGGTGGAGACGGGTGGGGGGTGGGTACAGGCTGGGGGCTCTCTGAGA
CTCTCCTGTGCAGCCTCTGGACGCGCCGCCAGTGGAAATGCCATGGCCTGGTTCCGC
CAGGCTCCAGGAAAGGAGCGTGAGTTTGTAGCATTGATTAGTTGGAGTGGTGGTCGC
CCATACTATGCAAACTCCGTGAAGGGCCGATTCGCCATCTCCAGAGACAACGCCACG
AATACGGTGTATCTGCAAATGAACAGACTGAAACCTGAGGACACGGCCGTTTATTAC
TGTGCAGCGTCGCCTACCATAGCGATACTACCTACTCCGTATGACTACTGGGGCCAG
GGGACCCAGGTCACCGTCTCCTCAGAACCCAAGACACCAAAACCACAA
JMO-B9,
SEQ ID NO: 81
QVQLVETGGGLVQAGASLRLSCAASGRTFSTDHMGWFRQAPQKEREFVAAINAWSGL
SIYYADSVKGRFTISRDNDKKTAYLQMNSLKPEDTAVYYCAAKEMGRGWVPQSSDDY
DAWGQGTQVTVSSEPKTPKPQ
SEQ ID NO: 82
CAGGTGCAGCTGGTGGAGACGGGGGGAGGATTGGTGCAGGCTGGGGCCTCTCTGAGA
CTCTCCTGTGCAGCCTCTGGACGCACCTTCAGTACCGATCACATGGGCTGGTTCCGC
CAGGCTCCACAGAAGGAGCGTGAGTTTGTGGCAGCAATAAATGCATGGAGTGGACTC
AGCATTTACTATGCAGACTCCGTGAAGGGCCGATTCACCATCTCCAGAGACAACGAC
AAGAAAACGGCATATCTACAAATGAACAGCCTGAAACCTGAGGACACGGCCGTTTAT
TACTGTGCAGCCAAGGAGATGGGTAGGGGTTGGGTGCCACAGAGCTCAGACGACTAT
GACGCCTGGGGCCAGGGGACCCAGGTCACCGTCTCCTCAGAACCCAAGACACCAAAA
CCACAA
JMO-C1,
SEQ ID NO: 83
QVQLVETGGGLVQAGGSLRLSCAVSGRTESSYAMAWFRQAPGKERDFVAAISWSGGA
PHYEDSVKGRFTISRDNAKNMVYLQMNSLKPDDTAVYYCAAAKAGYYSGSYYVGGGM
YDYWGQGTQVTVSSEPKTPKPQ
SEQ ID NO: 84
CAGGTGCAGCTGGTGGAGACTGGAGGAGGATTGGTGCAGGCTGGGGGCTCTCTGAGA
CTCTCCTGTGCAGTCTCTGGACGCACCTTCAGTAGCTATGCCATGGCCTGGTTCCGC
CAGGCTCCAGGGAAGGAGCGTGATTTTGTAGCAGCTATTAGCTGGAGTGGTGGTGCC
CCACACTATGAAGACTCCGTGAAGGGCCGATTCACCATCTCCAGAGACAACGCCAAG
AACATGGTATATCTCCAAATGAACAGCCTGAAACCTGACGACACGGCCGTTTACTAC
TGTGCAGCAGCGAAAGCAGGATACTATAGTGGTAGTTACTACGTGGGGGGGGGTATG
TATGACTACTGGGGCCAGGGGACCCAGGTCACCGTCTCCTCAGAACCCAAGACACCA
AAACCACAA
JMO-C10,
SEQ ID NO: 85
QVQLVETGGLVQAGGSLRLSCAASGSIGRVDNMGWYRQTPGKERERVAIITGGGTAI
YADTVKGRFTVSRDNAKNTIYLQMNSVKPEDTAVYFCNADISRSIESIVYRSYWGQG
TQVTVSSEPKTPKPQ
SEQ ID NO: 86
CAGGTGCAGCTGGTGGAGACAGGAGGCTTGGTGCAGGCTGGGGGGTCTCTGAGACTC
TCCTGTGCAGCCTCCGGAAGCATCGGCAGGGTCGATAACATGGGCTGGTACCGCCAA
ACTCCAGGGAAAGAGCGCGAGCGGGTCGCAATCATTACTGGAGGCGGTACCGCGATC
TATGCAGACACCGTGAAGGGCCGATTCACCGTCTCCAGAGACAACGCCAAGAACACA
ATATATCTACAAATGAACAGCGTGAAACCTGAGGACACAGCCGTCTATTTCTGTAAT
GCCGACATCAGTCGTAGTATTGAGTCCATCGTCTATCGTTCCTACTGGGGCCAGGGG
ACCCAGGTCACCGTCTCCTCAGAACCCAAGACACCAAAACCACAA
JMO-F4,
SEQ ID NO: 87
QVQLVETGGGLVQPGGSLRLSCAASGNIFSINAMGWYRQAPGKQRELVAAISNSGST
NYEDSVKGRFTVSRDNAKNTVYLQMNSLKPEDTAVYYCNAFDLVAGTRLGSWGQGTQ
VTVSSEPKTPKPQ
SEQ ID NO: 88
CAGGTGCAGCTGGTGGAGACGGGGGGAGGCTTGGTGCAGCCTGGGGGGTCTCTGAGA
CTCTCCTGTGCAGCCTCTGGAAACATCTTCAGTATCAATGCCATGGGCTGGTACCGC
CAGGCTCCAGGGAAGCAGCGCGAGTTGGTCGCAGCTATTAGTAATAGTGGTAGCACA
AACTATGAAGACTCCGTGAAGGGCCGATTCACCGTCTCCAGAGACAACGCCAAGAAC
ACGGTGTATCTGCAAATGAACAGCCTGAAACCTGAGGACACGGCCGTCTATTACTGT
AATGCCTTCGATTTAGTAGCTGGTACTAGGCTGGGGTCCTGGGGCCAGGGGACCCAG
GTCACCGTCTCCTCGGAACCCAAGACACCAAAACCACAA
JMO-F12,
SEQ ID NO: 89
QVQLVESGGGLVQPGGSLRLSCAASEFTLEHAAVGWFRQAPGKEREGVSCISSRDSN
TYYADSVKGRFTISRDNAENTVYLQMNSLKPEDTAVYYCATDVPCWDGSNWSLGHEY
DYWGQGTQVTVSSEPKTPKPQ
SEQ ID NO: 90
CAGGTGCAGCTGGTGGAGTCGGGGGGAGGCTTGGTGCAGCCTGGGGGGTCTCTGAGA
CTCTCCTGTGCAGCCTCTGAATTCACTTTGGAACATGCCGCCGTAGGCTGGTTCCGC
CAGGCCCCAGGGAAGGAGCGCGAGGGGGTCTCTTGTATTAGTAGTCGTGATAGTAAC
ACATACTATGCAGACTCCGTGAAGGGCCGATTCACCATCTCCAGAGACAATGCCGAA
AACACGGTATATCTGCAAATGAACAGCCTGAAACCTGAGGACACGGCCGTTTATTAC
TGTGCGACAGATGTCCCCTGCTGGGACGGTAGTAACTGGTCCCTCGGTCATGAGTAT
GACTACTGGGGCCAGGGGACCCAGGTCACCGTCTCCTCAGAACCCAAGACACCAAAA
CCACAA

### New Anthrax EF/LF-Binding (Cross-Specific) VHHs

JMO-G1,
SEQ ID NO: 91
QVQLVETGGGLVQPGGSLRLSCAASGSISSINAMGWYRQAPGKQRELVAAITIRGNT
VYGDSVKGRFTVSRDNAKNTVYLQMNSLKPEDTAVYYCNAKSTPSLYAAGYGVDYWG
EGTLVTVSSEPKTPKPQ
SEQ ID NO: 92
CAGGTGCAGCTGGTGGAGACGGGGGGAGGCTTGGTGCAGCCTGGGGGGTCTCTGAGA
CTCTCCTGTGCAGCCTCTGGAAGCATCTCCAGTATCAATGCCATGGGCTGGTACCGC
CAGGCTCCAGGGAAGCAGCGCGAGTTGGTCGCGGCTATTACTATTCGTGGTAACACA
GTCTATGGAGACTCCGTGAAGGGCCGATTCACCGTCTCCAGAGACAACGCCAAGAAC
ACGGTGTATCTGCAAATGAACAGCCTGAAACCTGAGGACACGGCCGTCTATTACTGT
AATGCCAAGTCGACCCCGAGCTTGTACGCCGCCGGCTACGGCGTGGACTACTGGGGC
GAAGGGACCCTAGTCACCGTCTCCTCAGAACCCAAGACACCAAAACCACAA
JMO-C9,
SEQ ID NO: 93
QVQLVETGGGLVQAGGSLRLSCAASGNISSINAMAWYRQAPGQQRELVAGITSGGRT
QYTDSVKGRFTISRDNAKNTVYLQMESLKPEDTAVYYCNAKSPPSTWATGGGMNYWG
KGTLVTVSSEPKTPKPQ
SEQ ID NO: 94
CAGGTGCAGCTGGTGGAGACGGGGGGAGGCTTGGTGCAGGCTGGGGGGTCTCTGAGA
CTCTCCTGTGCAGCCTCTGGGAACATCTCCAGTATCAATGCCATGGCCTGGTACCGC
CAGGCTCCAGGGCAGCAGCGCGAGCTGGTCGCAGGGATTACTAGTGGTGGCAGGACA
CAATATACAGACTCCGTGAAGGGCCGATTCACCATCTCCAGAGACAACGCCAAGAAC
ACGGTGTATCTGCAAATGGAGAGTCTGAAACCTGAGGACACAGCCGTCTATTACTGT
AATGCAAAAAGCCCTCCCAGTACCTGGGCCACGGGGGGGGGCATGAACTACTGGGGC
AAAGGGACCCTGGTCACCGTCTCCTCAGAACCCAAGACACCAAAACCACAA
JMN-D10,
SEQ ID NO: 95
QVQLVETGGALVQAGGSLRLSCAASETSSVSLSWMGWYRQAPGKERELVAGINRDRP
KYKESVKGRFTISRDNAQNTVYLQMNSLKPEDTAVYYCNTVPPRGDYWGQGTQVTVS
SEPKTPKPQ
SEQ ID NO: 96
CAGGTGCAGCTGGTGGAGACAGGAGGAGCCTTGGTGCAGGCGGGGGGGTCTCTGAGA
CTCTCCTGTGCAGCCTCTGAGACATCTTCAGTATCGCTATCATGGATGGGCTGGTAC
CGCCAGGCTCCTGGGAAGGAGCGCGAGTTGGTCGCAGGCATTAATCGTGATAGGCCA
AAGTATAAAGAGTCCGTGAAGGGCCGATTCACCATCTCCAGAGACAACGCCCAGAAT
ACGGTGTATCTGCAAATGAACAGCCTGAAACCTGAGGACACAGCCGTCTATTACTGT
AATACGGTTCCACCACGCGGCGACTACTGGGGCCAGGGGACCCAGGTCACCGTCTCC
TCAGAACCCAAGACACCAAAACCACAA
JMN-E12,
SEQ ID NO: 97
QVQLVESGGGLVQPGGSLRVSCVASGNISSVAAMAWYRQRPEKRRELVAVITNSGGT
AYTDSVRGRFTISRDNVKSTVYLQMNNLKPEDTAVYYCNARGLDAGSGRIDYWGQGT
QVTVSSEPKTPKPQ
SEQ ID NO: 98
CAGGTGCAGCTGGTGGAGTCCGGTGGAGGCTTGGTGCAGCCTGGGGGGTCTCTGAGA
GTCTCCTGTGTAGCCTCTGGAAACATCTCCAGTGTCGCTGCCATGGCCTGGTACCGC
CAGAGACCAGAGAAGCGCCGCGAATTGGTCGCAGTCATTACTAACAGCGGTGGCACA
GCCTATACAGACTCCGTGAGGGGCCGATTCACCATCTCCAGAGACAATGTCAAGTCA
ACGGTGTATCTACAAATGAATAACCTGAAACCTGAGGACACAGCCGTGTATTACTGT
AATGCGAGGGGGTTAGACGCCGGGTCAGGGCGCATTGACTACTGGGGCCAGGGAACC
CAGGTCACCGTCTCCTCAGAACCCAAGACACCAAAACCACAA
JMN-F1,
SEQ ID NO: 99
QVQLVESGGGLAQTGGSLNLSCAASGPTFSGYGMGWFRQAPGKEREFLAVIRWSVGN
TLYAESVKGRFTISRDKVKNTGYLQIDNLKPEDTAVYYCAAGAYVTTRSRDYAYWGQ
GTQVTVSSEPKTPKPQ
SEQ ID NO: 100
CAGGTGCAGCTGGTGGAGTCGGGGGGAGGATTGGCGCAGACTGGGGGCTCTCTGAAC
CTCTCCTGTGCAGCCTCTGGACCGACTTTCAGCGGCTATGGTATGGGCTGGTTCCGC
CAGGCTCCAGGGAAGGAGCGTGAATTTCTAGCGGTAATTCGCTGGAGTGTAGGTAAT
ACATTGTATGCAGAGTCCGTCAAGGGCCGATTCACCATCTCCAGAGACAAGGTCAAG
AACACGGGGTATCTGCAAATAGACAACCTGAAACCCGAGGACACGGCCGTTTATTAC
TGTGCAGCGGGGGCGTACGTAACTACGAGGTCCCGCGACTATGCCTACTGGGGCCAG
GGGACCCAGGTCACCGTCTCCTCAGAACCCAAGACACCAAAACCACAA
JLO-A4,
SEQ ID NO: 101
QVQLVETGGRQVQTGDSLNLSCAASEHTFSPKVMGWERQAPGKGREFVATITIRGGR
TLYADSVKGRFAISKDGAKNTVYLQMNSLKPEDTAVYYCAASRELAIPPTQPSAYDH
WGQGTQVTVSSAHHSEDPS
SEQ ID NO: 102
CAGGTGCAGCTCGTGGAGACCGGCGGACGTCAGGTGCAGACTGGGGACTCTCTGAAC
CTCTCTTGCGCAGCTTCTGAACACACCTTCAGTCCTAAAGTTATGGGGTGGTTCCGC
CAGGCTCCAGGCAAGGGGCGTGAGTTTGTAGCAACTATCACAATCCGTGGCGGTCGC
ACACTCTATGCAGATTCCGTGAAGGGCCGATTTGCCATCTCCAAAGACGGCGCCAAG
AATACGGTGTATCTGCAAATGAACAGTCTGAAACCTGAGGACACGGCCGTTTATTAC
TGTGCAGCAAGTCGTGAGCTAGCGATACCACCGACGCAGCCTTCGGCATACGACCAC
TGGGGCCAGGGGACCCAGGTCACCGTCTCCTCAGCGCACCACAGCGAAGACCCCTCG

### Appendix D

Included in Appendix D are 2 anthrax PA-binding VNAs

### New Anthrax PA-Binding VNAs

VNA1-PA (JKD-11)
SEQ ID NO: 103
QVQLAESGGGLVQPGGSLGLSCVVASERSINNYGMGWYRQAPGKQRELVAQISSGGT
TNYADSVEGRFTISRDNVKKMVHLQVNSLKPEDTAVYYCNSLLRTFSWGQGTQVTVS
SEPKTPKPQAIAGGGGSGGGGSGGGGSLQGQVQLVESGGGLVQPGGSLSVSCAASGS
IARPGAMAWYRQAPGKERELVASITPGGLTNYADSVTGRFTISRDNAKRTVYLQMNS
LQPEDTAVYYCHARIIPLGLGSEYRDHWGQGTQVTVSSAHHSEDPS
SEQ ID NO: 104
CAGGTGCAGCTGGCGGAGTCGGGCGGAGGCTTGGTGCAGCCTGGGGGGTCTCTGGGA
CTCTCCTGTGTAGTCGCCTCTGAAAGAAGCATCAATAATTATGGCATGGGCTGGTAC
CGCCAGGCTCCAGGGAAGCAGCGCGAGTTGGTCGCGCAAATTAGTAGTGGTGGTACC
ACAAATTATGCAGACTCCGTAGAGGGCCGATTCACCATCTCCAGAGACAACGTCAAG
AAAATGGTGCATCTTCAAGTGAACAGCCTGAAACCTGAGGACACGGCCGTCTATTAC
TGTAATTCGCTACTCCGAACTTTTTCCTGGGGCCAGGGGACCCAGGTCACCGTCTCC
TCGGAACCCAAGACACCAAAACCACAAGCGATCGCTGGTGGAGGCGGTTCAGGCGGA
GGTGGCTCTGGCGGTGGCGGTTCCCTGCAGGGTCAGKTGCAGCTSGYGGAGTCCGGG
GGCGGCTTGGTGCAGCCCGGGGGGTCTCTGAGTGTCTCCTGTGCAGCCTCTGGAAGC
ATCGCAAGACCAGGTGCCATGGCCTGGTACCGCCAGGCTCCAGGGAAGGAGCGCGAG
TTGGTCGCGTCTATTACGCCTGGTGGTCTTACAAACTATGCGGACTCCGTGACGGGC
CGATTCACCATTTCCAGAGACAACGCCAAGAGGACGGTGTATCTGCAGATGAACAGC
CTCCAACCCGAGGACACGGCCGTCTATTACTGTCATGCACGAATAATTCCCCTAGGA
CTTGGGTCCGAATACAGGGACCACTGGGGCCAGGGGACTCAGGTCACCGTCTCCTCA
GCGCACCACAGCGAAGACCCCTCG
VNA2-PA (JKU-1)
SEQ ID NO: 105
QVQLAESGGGLVQPGGSLGLSCVVASERSINNYGMGWYRQAPGKQRELVAQISSGGT
TNYADSVEGRETISRDNVKKMVHLQVNSLKPEDTAVYYCNSLLRTFSWGQGTQVTVS
SEPKTPKPQAIAGGGGGGGGSGGGGSLQGQVQLAESGGGGLVQAGGSLRLSCAASG
RTFSGYAMGWFRQAPGKEREFVADISWSGHNTYYGDSVKGRFTISRDTAKNTVYLQM
NSLKPEDTAVYYCAAEGARTHLSDSYYFPGLWAEPPVGYWGQGTQVTVSSEPKTPKP
SEQ ID NO: 106
CAGGTGCAGCTGGCGGAGTCGGGCGGAGGCTTGGTGCAGCCTGGGGGGTCTCTGGGA
CTCTCCTGTGTAGTCGCCTCTGAAAGAAGCATCAATAATTATGGCATGGGCTGGTAC
CGCCAGGCTCCAGGGAAGCAGCGCGAGTTGGTCGCGCAAATTAGTAGTGGTGGTACC
ACAAATTATGCAGACTCCGTAGAGGGCCGATTCACCATCTCCAGAGACAACGTCAAG
AAAATGGTGCATCTTCAAGTGAACAGCCTGAAACCTGAGGACACGGCCGTCTATTAC
TGTAATTCGCTACTCCGAACTTTTTCCTGGGGCCAGGGGACCCAGGTCACCGTCTCC
TCGGAACCCAAGACACCAAAACCACAAGCGATCGCTGGTGGAGGCGGTTCAGGCGGA
GGTGGCTCTGGCGGTGGCGGTTCCCTGCAGGGTCAGGTGCAGCTCGCGGAGTCGGGT
GGGGGAGGACTGGTGCAGGCTGGGGGCTCTCTGAGACTCTCCTGTGCAGCCTCTGGA
CGCACCTTCAGTGGCTATGCCATGGGCTGGTTCCGCCAGGCTCCGGGGAAGGAGCGT
GAGTTTGTAGCCGATATTAGCTGGAGTGGTCATAACACGTACTATGGAGACTCCGTG
AAGGGCCGATTCACCATCTCCAGAGACACCGCCAAGAACACGGTGTATCTGCAAATG
AACAGCCTGAAACCTGAGGACACGGCCGTTTATTACTGTGCAGCGGAGGGGGCCCGT
ACACACCTTAGTGATAGTTACTACTTCCCGGGCCTCTGGGCCGAACCCCCCGTGGGC
TACTGGGGCCAGGGGACCCAGGTCACTGTCTCCTCAGAACCCAAGACACCAAAACCA
CAA

### Appendix E

Included in Appendix E are 10 BoNT/B-protease light chain (BLc) binding VHHs; 2 BoNT/E-protease light chain (ELc) binding VHHs; 4 BoNT/B-binding VHH heterodimers; and 3 BoNT/E-binding VHH heterodimers.

### New BoNT/B-Protease Light Chain (BLc) Binding VHHs

JLS-G8,
SEQ ID NO: 107
QVQLVESGGGSVQAGGSLRLTCTGSGRSFALYYMAWFRQAPGKEREFVAAISHNSLSAIV
ADSLKGRFTISRDNARNQVVLQMNSLKPEDTAVYYCAADFSPSTYNTNYYRTGSYQYWGQ
GTQVTVSSEPKTPKPQ
SEQ ID NO: 108
CAGGTGCAGCTGGTGGAGTCGGGGGGAGGATCGGTGCAGGCTGGGGGCTCTCTGAGACTC
ACCTGTACAGGCTCTGGACGCAGTTTCGCGCTCTATTACATGGCCTGGTTCCGCCAGGCT
CCAGGGAAGGAGCGTGAGTTTGTAGCAGCTATCAGCCACAATTCGTTAAGCGCAATCGTT
GCAGACTCCCTAAAGGGCCGATTCACCATCTCCAGAGACAACGCCAGAAACCAGGTGGTT
CTACAAATGAACAGCCTGAAACCTGAGGACACGGCCGTTTATTACTGTGCAGCAGACTTT
TCGCCCTCGACCTATAATACAAATTACTACCGCACCGGTTCGTATCAGTATTGGGGCCAG
GGGACCCAGGTCACCGTCTCCTCAGAACCCAAGACACCAAAACCACAA
JND-A12,
SEQ ID NO: 109
QVQLVETGGGLVQAGGSLGLSCAASGLSFNWYDVGWFRQAPGKEREFVASRSSGGGSTYY
GDSVKGRESISTDNAKNTAYLQMNSLKPEDTAVYYCAADWTGRAGESVGYYRPDEYDYWG
QGTQVTVSEEPKTPKPQ
SEQ ID NO: 110
CAGGTGCAGCTGGTGGAGACGGGAGGAGGATTGGTGCAGGCTGGGGGCTCTCTGGGACTC
TCCTGTGCAGCCTCTGGACTGTCCTTTAATTGGTATGACGTGGGCTGGTTCCGCCAGGCT
CCAGGGAAGGAGCGTGAGTTTGTAGCGTCTCGTAGCTCGGGTGGTGGTAGTACATATTAT
GGAGACTCCGTGAAGGGCCGATTCAGCATCTCCACAGACAATGCCAAGAACACGGCGTAT
CTGCAAATGAACAGCCTAAAACCTGAGGACACGGCCGTTTACTACTGTGCAGCAGATTGG
ACAGGCCGCGCAGGCTTCAGTGTTGGTTACTACCGGCCCGATGAGTATGACTACTGGGGC
CAGGGGACCCAGGTCACCGTCTCCGAAGAACCCAAGACACCAAAACCACAA
JND-B4,
SEQ ID NO: 111
QVQLVETGGGLVQPGGSLRLSCVASGFTLDSYAIGWFRQAPGKEREGVSCMSSGDGSTYY
TNSVKGRFTISRDNAQNTVYLQMNSLKPEDTAVYYCAADGFDYCSAYVPGRGMNYSGKGT
LVTVSSEPKTPKPQ
SEQ ID NO: 112
CAGGTGCAGCTGGTGGAGACGGGGGGAGGCTTGGTGCAGCCTGGGGGGTCTCTGAGACTC
TCCTGTGTAGCCTCTGGATTCACTTTGGATTCATATGCCATAGGCTGGTTCCGCCAGGCC
CCAGGGAAGGAGCGTGAGGGGGTCTCATGTATGAGTAGTGGTGATGGTAGCACATACTAT
ACAAACTCCGTGAAGGGCCGATTCACCATCTCCAGAGACAACGCCCAGAACACGGTGTAT
CTGCAAATGAACAGCCTGAAACCTGAGGACACAGCCGTTTATTACTGTGCAGCAGATGGG
TTTGACTATTGTTCAGCTTATGTGCCCGGGAGAGGCATGAACTACTCGGGCAAAGGGACC
CTGGTCACCGTCTCCTCAGAACCCAAGACACCAAAACCACAA
JND-C7,
SEQ ID NO: 113
QVQLVETGGGLVQPGGSLRLSCAGSGFTLDNYAVGWFRQAPGKEREGVSCISSSDDNTDY
SDSVKGRFTISRDNAKDTVYLQMNSLKPEDTAIYYCAAESPTFGFSCTVATDPYDYWGQG
TQVTVSSEPKTPKPQ
SEQ ID NO: 114
CAGGTGCAGCTGGTGGAGACGGGTGGAGGCTTGGTGCAGCCTGGGGGGTCTCTGAGACTC
TCCTGTGCAGGCTCTGGATTCACTTTGGATAATTATGCCGTCGGCTGGTTCCGCCAGGCC
CCAGGGAAGGAGCGTGAGGGGGTCTCATGTATTAGTAGTAGTGATGATAACACTGACTAT
TCAGACTCCGTGAAGGGCCGATTCACCATCTCCAGAGACAACGCCAAGGACACGGTCTAT
CTGCAAATGAACAGCCTGAAACCTGAGGACACAGCGATTTATTACTGTGCAGCAGAAAGC
CCGACGTTCGGGTTCAGCTGTACGGTAGCCACTGATCCATATGACTACTGGGGCCAGGGG
ACCCAGGTCACCGTCTCCTCAGAACCCAAGACACCAAAACCACAA
JND-E4,
SEQ ID NO: 115
QVQLVETGGGLVQPGGSLRLSCAASGFTLDGYAAGWFRQAPGKERELVSWISSTDGSTYY
AASVKGRFTVSRDNAKNTVYLQMNSLKPEDTAVYYCTAGLGLDVSDYVYDYWGQGTQVTV
SSEPKTPKPQ
SEQ ID NO: 116
CAGGTGCAGCTGGTGGAGACGGGGGGAGGCTTGGTGCAGCCTGGGGGGTCTCTGAGGCTC
TCCTGTGCAGCCTCTGGATTCACTTTGGATGGCTATGCCGCAGGCTGGTTCCGCCAGGCC
CCAGGGAAGGAGCGTGAGTTGGTCTCATGGATTAGTAGCACTGATGGTAGCACATACTAT
GCAGCCTCCGTGAAGGGCCGATTCACCGTCTCCAGAGACAACGCCAAGAACACGGTGTAT
CTACAAATGAACAGCCTGAAACCTGAGGACACAGCCGTTTATTACTGTACAGCAGGTCTA
GGGCTTGACGTTAGCGACTATGTATATGACTACTGGGGCCAGGGGACCCAGGTCACCGTC
TCCTCAGAACCCAAGACACCAAAACCACAA
JND-E5,
SEQ ID NO: 117
QVQLVESGGLVQPGGSLRLSCAASGFTLDYYGIGWVRQAPGKEREEVSCITSGGLTNYPD
SVKGRFTISRDNAKNTVYLQMNSLKPEDTAVYYCAIDRVGVCAMEDFGSWGQGTQVTVSS
EPKTPKPQ
SEQ ID NO: 118
CAGGTGCAGCTGGTGGAGTCGGGCGGCTTGGTGCAGCCTGGGGGGTCTCTGAGACTCTCC
TGTGCAGCCTCTGGATTCACTTTGGATTATTATGGCATAGGCTGGGTCCGCCAGGCCCCA
GGGAAGGAGCGTGAGGAGGTCTCATGTATTACTAGTGGTGGTCTCACAAACTATCCAGAC
TCCGTGAAGGGCCGATTCACCATCTCCAGAGACAACGCCAAGAACACAGTGTATCTGCAA
ATGAACAGCCTGAAACCTGAGGACACGGCCGTTTATTACTGTGCAATCGACCGTGTGGGA
GTATGCGCGATGGAGGACTTTGGTTCCTGGGGCCAGGGGACCCAGGTCACCGTCTCCTCG
GAACCCAAGACACCAAAACCACAA
JND-E9,
SEQ ID NO: 119
QVQLVETGGGLVQAGDSLRLSCAASGRTENYYAMAWFRQAPGKEREFVAFINWSGDSTYY
AGSVKGRFTISRDNAKNTVYLQMNNLKPEDTAVYSCAAEFGTFSYLQGDDYSYWGQGTQV
TVSSEPKTPKPQ
SEQ ID NO: 120
CAGGTGCAGCTGGTGGAGACAGGTGGAGGATTGGTGCAGGCTGGGGACTCTCTGAGACTC
TCCTGTGCAGCCTCTGGACGCACCTTCAATTACTATGCCATGGCCTGGTTCCGCCAGGCC
CCAGGAAAGGAGCGTGAATTTGTAGCATTTATTAACTGGAGCGGCGATAGTACATACTAT
GCAGGCTCCGTGAAGGGCCGATTCACCATCTCCAGAGACAACGCCAAGAACACGGTGTAT
CTGCAAATGAACAACCTGAAACCTGAGGACACGGCCGTTTATTCCTGTGCAGCAGAATTC
GGTACATTTTCCTACTTGCAAGGCGATGACTATAGCTACTGGGGCCAGGGGACCCAGGTC
ACCGTCTCCTCAGAACCCAAGACACCAAAACCACAA
JND-F3,
SEQ ID NO: 121
QVQLVESGGGLVQAGGSLRLSCAASGRSFSSYRMGWFRQAPGKERELVAGISWSGSSTWY
ADSVKGRFTISRDNAKNTVYLQMNSLKPEDTAVYYCAADGLGTDWSDAIWDYWGQGTQVT
VSSEPKTPKPQ
SEQ ID NO: 122
CAGGTGCAGCTGGTGGAGTCTGGAGGAGGATTGGTGCAGGCTGGGGGCTCTCTGAGACTC
TCCTGTGCAGCCTCTGGACGCAGCTTCAGTAGCTATCGCATGGGCTGGTTCCGCCAGGCT
CCAGGGAAGGAGCGTGAGCTTGTAGCAGGTATTAGCTGGAGTGGAAGTAGTACATGGTAT
GCAGACTCCGTGAAGGGCCGATTCACCATCTCCAGAGACAACGCCAAGAACACGGTGTAT
CTGCAAATGAACAGCCTGAAACCCGAGGACACGGCCGTTTATTACTGTGCAGCAGATGGG
CTAGGGACGGATTGGAGCGATGCCATATGGGACTACTGGGGCCAGGGGACCCAGGTCACC
GTCTCCTCAGAACCCAAGACACCAAAACCACAA
JND-F7,
SEQ ID NO: 123
QVQLVESGGGLVQAGGSLRLSCAASGRNFSHYAMGWFRQAPGKAREFVATINRDGDSTYY
TNSVKGRFTISRENAKNTGYLQMNSLKPEDTAVYYCGVQYSWSGTSIYWREYEYAYWGQG
AQVTVSSEPKTPKPQ
SEQ ID NO: 124
CAGGTGCAGCTGGTGGAGTCGGGGGGAGGATTGGTGCAGGCTGGGGGCTCTCTGAGACTC
TCCTGTGCAGCCTCTGGACGCAATTTCAGTCACTATGCCATGGGCTGGTTCCGCCAGGCT
CCAGGGAAGGCGCGTGAGTTTGTAGCAACTATTAACCGGGATGGTGATAGCACATACTAT
ACGAACTCCGTGAAGGGCCGATTCACCATCTCCAGAGAGAACGCCAAGAACACGGGATAT
CTGCAAATGAACAGCCTGAAACCTGAGGACACGGCCGTTTATTACTGTGGAGTACAATAC
TCGTGGTCGGGTACAAGTATTTACTGGAGGGAGTATGAGTATGCCTACTGGGGCCAGGGG
GCCCAGGTCACCGTCTCCTCAGAACCCAAGACACCAAAACCACAA
JNE-B10,
SEQ ID NO: 125
QVQLVESGGGLVQPGGSLRLSCAASGFPFHAYYMSWVRQAPGKGLEWVSHIGNGGIITRY
ADSVKGRFTISRDNAKNTLYLQMTNLKPEDTALYYCTLGTRDDLGPERGQGTQVTVSSEP
KTPKPQ
SEQ ID NO: 126
CAGGTGCAGCTGGTGGAGTCGGGTGGAGGCTTGGTGCAGCCTGGGGGGTCTCTGAGACTC
TCCTGTGCAGCCTCTGGATTCCCCTTCCATGCCTACTACATGAGCTGGGTCCGCCAGGCT
CCAGGAAAGGGGCTCGAGTGGGTCTCCCATATTGGCAATGGTGGTATTATTACACGCTAT
GCAGACTCCGTGAAGGGCCGGTTCACCATCTCCAGAGACAACGCCAAGAACACGCTGTAT
CTGCAAATGACCAACCTGAAACCTGAGGACACGGCCCTGTATTATTGTACCCTGGGGACC
CGCGACGACCTGGGGCCTGAGAGGGGCCAGGGGACCCAGGTCACCGTCTCCTCAGAACCC
AAGACACCAAAACCACAA

### New BoNT/E-Protease Light Chain (ELc) Binding VHHs

JNB-B12,
SEQ ID NO: 127
QVQLVESGGGLVQPGGSLRLSCAASEGIFSVDAMGWYRQVPGKQRELVARITRGGSIIYA
DSVKGRFTISRDSAKNTVYLQMNSLKPEDTAVYYCNRLYRGTLTFGQGTQVTVSSAHHSE
DPS
SEQ ID NO: 128
CAGGTGCAGCTCGTGGAGTCGGGTGGAGGCTTGGTGCAGCCTGGGGGGTCTCTGAGACTC
TCCTGTGCAGCCTCTGAGGGAATCTTCAGTGTTGATGCCATGGGCTGGTACCGCCAGGTT
CCAGGGAAGCAGCGCGAGTTGGTCGCACGAATTACCCGTGGTGGTAGCATAATTTATGCA
GACTCCGTGAAGGGCCGATTCACCATCTCCAGAGACAGCGCCAAGAACACGGTGTATCTG
CAAATGAACAGCCTGAAACCTGAGGACACGGCCGTCTATTACTGTAATCGCCTTTATAGG
GGTACCCTAACGTTCGGCCAGGGGACCCAGGTCACCGTCTCCTCAGCGCACCACAGCGAA
GACCCCTCG
JNC-D5,
SEQ ID NO: 129
QVQLVETGGGLVQAGGSLRLSCAASGRTESIYAMGWFRQAPGREREFVASISRMGWSTYY
GDSVKGRFTASRDNAKNTLYLQMNSLELEDTAVYFCAASASALRVNQWDYWGQGTQVTVS
SEPKTPKPQ
SEQ ID NO: 130
CAGGTGCAGCTGGTGGAGACCGGCGGAGGATTGGTGCAGGCTGGGGGCTCTCTGAGACTC
TCCTGTGCAGCCTCTGGACGCACCTTCAGTATCTATGCCATGGGCTGGTTCCGCCAGGCT
CCAGGGAGGGAGCGTGAGTTTGTAGCGTCTATTAGTCGGATGGGTTGGAGCACATATTAT
GGGGACTCCGTGAAGGGCCGATTCACCGCCTCCAGAGACAACGCCAAGAACACGCTGTAT
CTACAAATGAACAGCCTCGAACTTGAGGACACGGCCGTATATTTTTGTGCGGCATCTGCG
AGTGCGTTACGAGTTAATCAGTGGGACTACTGGGGCCAGGGGACCCAGGTCACCGTCTCC
TCAGAACCCAAGACACCAAAACCACAA

### New BoNT/B-Binding VHH Heterodimers

JLU-D10/JLI-G10,
SEQ ID NO: 131
QVQLVESGGGLVQPGGSLRLSCAASGFTLDSYAIGWFRQAPGKEREGVACISASGSGTDY
VDSVKGRFTVSRDQAKSMVFLQMNNMKPEDAAVYYCAADYRPRPLPIQAPCTMTGGNYWG
QGTQVTVSSEPKTPKPQAIAGGGGSGGGGSGGGGSLQGQVQLVESGGGLVQAGGSLRLSC
AASILTYDLDYYYIGWVRQAPGKEREGVSCISSTDGATYYADSVKGRFTISRNNAKNTVY
LQMNNLKPEDTAIYYCAAAPLAGRYCPASHEYGYWGQGTQVTVSSAHHSEDPS
SEQ ID NO: 132
CAGGTGCAGCTCGTGGAGTCAGGGGGAGGCTTGGTGCAGCCTGGGGGGTCTCTGAGACTC
TCCTGTGCAGCCTCTGGATTCACTTTAGATAGTTATGCAATAGGCTGGTTCCGCCAGGCC
CCAGGGAAGGAGCGTGAGGGGGTCGCATGTATTAGTGCTAGTGGTAGTGGCACGGACTAT
GTAGACTCCGTGAAGGGCCGATTCACCGTCTCCAGAGACCAGGCCAAGAGCATGGTGTTT
CTGCAAATGAACAACATGAAACCTGAGGACGCAGCCGTTTATTACTGTGCAGCAGATTAT
CGGCCGAGGCCCCTGCCGATTCAGGCGCCGTGTACAATGACAGGTGGCAACTACTGGGGC
CAGGGGACCCAGGTCACCGTCTCCTCAGAACCCAAGACACCAAAACCACAAGCGATCGCT
GGTGGAGGCGGTTCAGGCGGAGGTGGCTCTGGCGGTGGCGGTTCCCTGCAGGGTCAGGTG
CAGCTCGTGGAGTCCGGTGGAGGCTTGGTGCAGGCTGGGGGGTCTCTGAGACTCTCCTGT
GCAGCCTCTATACTCACTTATGATTTGGATTATTATTACATAGGCTGGGTCCGCCAGGCC
CCAGGGAAGGAGCGTGAGGGGGTCTCATGTATTAGTAGTACTGATGGTGCCACATACTAT
GCAGACTCCGTGAAGGGCCGATTCACCATCTCCAGAAACAACGCCAAGAACACGGTGTAT
CTGCAAATGAACAACCTAAAACCTGAGGACACAGCCATTTATTATTGTGCAGCAGCCCCC
CTGGCTGGGCGCTACTGTCCCGCCTCGCATGAGTATGGCTACTGGGGTCAGGGGACCCAG
GTCACCGTCTCGTCAGCGCACCACAGCGAAGACCCCTCG
JLU-D10/JLK-G12,
SEQ ID NO: 133
QVQLVESGGGLVQPGGSLRLSCAASGFTLDSYAIGWFRQAPGKEREGVACISASGSGTDY
VDSVKGRFTVSRDQAKSMVFLQMNNMKPEDAAVYYCAADYRPRPLPIQAPCTMTGGNYWG
QGTQVTVSSEPKTPKPQAIAGGGGSGGGGSGGGGSLQGQXQLXESGGGLVQAGGSLRLSC
AASEFRAEHFAVGWFRQAPGKEREGVSCVDASGDSTAYADSVKGRFTISRDNNKNVVYLQ
MDSLEPEDTGDYYCGASYFTVCAKSMRKIEYRYWGQGTQVTVSSEPKTPKPQ
SEQ ID NO: 134
CAGGTGCAGCTCGTGGAGTCAGGGGGAGGCTTGGTGCAGCCTGGGGGGTCTCTGAGACTC
TCCTGTGCAGCCTCTGGATTCACTTTAGATAGTTATGCAATAGGCTGGTTCCGCCAGGCC
CCAGGGAAGGAGCGTGAGGGGGTCGCATGTATTAGTGCTAGTGGTAGTGGCACGGACTAT
GTAGACTCCGTGAAGGGCCGATTCACCGTCTCCAGAGACCAGGCCAAGAGCATGGTGTTT
CTGCAAATGAACAACATGAAACCTGAGGACGCAGCCGTTTATTACTGTGCAGCAGATTAT
CGGCCGAGGCCCCTGCCGATTCAGGCGCCGTGTACAATGACAGGTGGCAACTACTGGGGC
CAGGGGACCCAGGTCACCGTCTCCTCAGAACCCAAGACACCAAAACCACAAGCGATCGCT
GGTGGAGGCGGTTCAGGCGGAGGTGGCTCTGGCGGTGGCGGTTCCCTGCAGGGTCAGKTG
CAGCTSGYGGAGTCCGGTGGAGGCTTGGTGCAGGCTGGGGGGTCTCTGAGACTCTCCTGT
GCAGCCTCTGAATTCCGTGCGGAGCATTTTGCCGTGGGCTGGTTCCGCCAGGCCCCAGGG
AAGGAGCGTGAGGGGGTCTCATGTGTAGACGCGAGTGGTGATAGTACAGCATATGCGGAC
TCTGTGAAGGGCCGATTCACCATCTCCAGAGACAACAACAAGAACGTAGTGTATCTGCAA
ATGGACAGCCTGGAACCTGAAGACACAGGAGATTATTATTGTGGAGCCTCGTACTTTACT
GTCTGCGCCAAGAGCATGCGGAAAATTGAATATAGGTACTGGGGCCAGGGGACCCAGGTC
ACCGTCTCCTCAGAACCCAAGACACCAAAACCACAA
JLI-G10/JLO-G11,
SEQ ID NO: 135
QVQLVESGGGLVQAGGSLRLSCAASILTYDLDYYYIGWVRQAPGKEREGVSCISSTDGAT
YYADSVKGRFTISRNNAKNTVYLQMNNLKPEDTAIYYCAAAPLAGRYCPASHEYGYWGQG
TQVTVSSAHHSEDPSAIAGGGGSGGGGSGGGGSLQGQVQLVESGGGLVQPGGSLRLSCEA
SGFHLEHFAVGWFRQAPGKEREGVSCISASGDSTTYADSVKGRSTISKDNAKNAVYLQMD
SLRPEDTGDYYCAASHFSVCGKNIRKIEYRYWGQGTPVTVSSEPKTPKPQ
SEQ ID NO: 136
CAGGTGCAGCTGGTGGAGTCCGGTGGAGGCTTGGTGCAGGCTGGGGGGTCTCTGAGACTC
TCCTGTGCAGCCTCTATACTCACTTATGATTTGGATTATTATTACATAGGCTGGGTCCGC
CAGGCCCCAGGGAAGGAGCGTGAGGGGGTCTCATGTATTAGTAGTACTGATGGTGCCACA
TACTATGCAGACTCCGTGAAGGGCCGATTCACCATCTCCAGAAACAACGCCAAGAACACG
GTGTATCTGCAAATGAACAACCTAAAACCTGAGGACACAGCCATTTATTATTGTGCAGCA
GCCCCCCTGGCTGGGCGCTACTGTCCCGCCTCGCATGAGTATGGCTACTGGGGTCAGGGG
ACCCAGGTCACCGTCTCGTCAGCGCACCACAGCGAAGACCCCTCGGCGATCGCTGGTGGA
GGCGGTTCAGGCGGAGGTGGCTCTGGCGGTGGCGGTTCCCTGCAGGGTCAGGTGCAGCTG
GTGGAGTCTGGTGGAGGCTTGGTGCAGCCTGGGGGGTCTCTGAGACTCTCCTGTGAAGCC
TCAGGATTCCATTTGGAGCATTTTGCCGTAGGCTGGTTCCGCCAGGCCCCAGGGAAGGAG
CGTGAGGGGGTCTCATGTATAAGCGCGAGTGGTGATAGTACAACGTATGCAGACTCCGTG
AAGGGCCGATCCACCATCTCCAAAGACAACGCCAAGAACGCGGTGTATCTGCAAATGGAC
AGCCTGAGACCCGAGGACACAGGCGATTATTACTGTGCAGCCTCGCACTTCAGTGTCTGC
GGCAAGAACATTCGGAAAATTGAGTATAGGTACTGGGGCCAGGGGACCCCGGTCACCGTC
TCCTCAGAACCCAAGACACCAAAACCACAA
JLI-G10/JLK-G12,
SEQ ID NO: 137
QVQLVESGGGLVQAGGSLRLSCAASILTYDLDYYYIGWVRQAPGKEREGVSCISSTDGAT
YYADSVKGRFTISRNNAKNTVYLQMNNLKPEDTAIYYCAAAPLAGRYCPASHEYGYWGQG
TQVTVSSAHHSEDPSAIAGGGGSGGGGSGGGGSLQGQVQLAESGGGLVQAGGSLRLSCAA
SEFRAEHFAVGWERQAPGKEREGVSCVDASGDSTAYADSVKGRFTISRDNNKNVVYLQMD
SLEPEDTGDYYCGASYFTVCAKSMRKIEYRYWGQGTQVTVSSEPKTPKPQ
SEQ ID NO: 138
CAGGTGCAGCTGGTGGAGTCCGGTGGAGGCTTGGTGCAGGCTGGGGGGTCTCTGAGACTC
TCCTGTGCAGCCTCTATACTCACTTATGATTTGGATTATTATTACATAGGCTGGGTCCGC
CAGGCCCCAGGGAAGGAGCGTGAGGGGGTCTCATGTATTAGTAGTACTGATGGTGCCACA
TACTATGCAGACTCCGTGAAGGGCCGATTCACCATCTCCAGAAACAACGCCAAGAACACG
GTGTATCTGCAAATGAACAACCTAAAACCTGAGGACACAGCCATTTATTATTGTGCAGCA
GCCCCCCTGGCTGGGCGCTACTGTCCCGCCTCGCATGAGTATGGCTACTGGGGTCAGGGG
ACCCAGGTCACCGTCTCGTCAGCGCACCACAGCGAAGACCCCTCGGCGATCGCTGGTGGA
GGCGGTTCAGGCGGAGGTGGCTCTGGCGGTGGCGGTTCCCTGCAGGGTCAGGTGCAGCTG
GCGGAGTCCGGTGGAGGCTTGGTGCAGGCTGGGGGGTCTCTGAGACTCTCCTGTGCAGCC
TCTGAATTCCGTGCGGAGCATTTTGCCGTGGGCTGGTTCCGCCAGGCCCCAGGGAAGGAG
CGTGAGGGGGTCTCATGTGTAGACGCGAGTGGTGATAGTACAGCATATGCGGACTCTGTG
AAGGGCCGATTCACCATCTCCAGAGACAACAACAAGAACGTAGTGTATCTGCAAATGGAC
AGCCTGGAACCTGAAGACACAGGAGATTATTATTGTGGAGCCTCGTACTTTACTGTCTGC
GCCAAGAGCATGCGGAAAATTGAATATAGGTACTGGGGCCAGGGGACCCAGGTCACCGTC
TCCTCAGAACCCAAGACACCAAAACCACAA

### New BoNT/E-Binding VHH Heterodimers

JLE-E5/JLE-E9,
SEQ ID NO: 139
QVQLVETGGGLVQAGGSLRLSCAASGRSYAMGWFRQGPGKEREFVATISWSSTNTWYADS
VKGRFTISRDNAKNTVYLQMNSLKPEDTAVYYCAASHRFSDYPMRSEDGMDYWGKGTLVT
VSSEPKTPKPQAIAGGGGSGGGGSGGGGSLQGQVQLVETGGGLVQAGGSLRLSCAASGRT
FSSYSMGWFRQAPGKEREYVAAVNSNGDSTFYADSIKGRFTVSRDAAKNTVYLQMNSLKP
EDTALYYCAAVYGRYTYQSPKSYEYWGQGTQVTVSSEPKTPKPQ
SEQ ID NO: 140
CAGGTGCAGCTGGTGGAGACGGGGGGAGGATTGGTGCAGGCTGGGGGCTCTCTGAGACTC
TCCTGTGCAGCCTCTGGACGCAGTTATGCCATGGGCTGGTTCCGCCAGGGTCCAGGGAAG
GAGCGTGAGTTTGTAGCCACTATCAGTTGGAGTAGTACTAACACATGGTATGCAGATTCC
GTGAAGGGCCGATTCACCATCTCTAGAGACAACGCCAAGAACACGGTGTATCTGCAAATG
AACAGCCTGAAACCTGAGGACACGGCTGTTTATTACTGTGCAGCGAGCCATCGTTTTAGC
GACTATCCCATGAGGTCAGAGGACGGCATGGACTACTGGGGCAAAGGGACCCTGGTCACC
GTCTCCTCAGAACCCAAGACACCAAAACCACAAGCGATCGCTGGTGGAGGCGGTTCAGGC
GGAGGTGGCTCTGGCGGTGGCGGTTCCCTGCAGGGTCAGGTGCAGCTGGTGGAGACGGGA
GGAGGATTGGTGCAGGCTGGGGGCTCTCTGAGACTCTCGTGTGCAGCCTCTGGACGCACC
TTCAGTAGCTATTCCATGGGCTGGTTCCGCCAGGCTCCAGGGAAGGAGCGTGAGTATGTA
GCAGCAGTTAACTCCAATGGCGACAGTACATTCTATGCCGACTCCATTAAGGGCCGATTC
ACCGTCTCCAGAGACGCCGCCAAGAACACAGTCTATCTGCAAATGAACAGCCTGAAACCT
GAGGACACGGCCCTTTATTACTGTGCAGCTGTCTACGGTAGATACACTTACCAGTCCCCA
AAATCGTATGAGTACTGGGGCCAGGGGACCCAGGTCACCGTCTCCTCAGAACCCAAGACA
CCAAAACCACAA
JLE-E5/JLE-G6,
SEQ ID NO: 141
QVQLVETGGGLVQAGGSLRLSCAASGRSYAMGWFRQGPGKEREFVATISWSSTNTWYADS
VKGRFTISRDNAKNTVYLQMNSLKPEDTAVYYCAASHRESDYPMRSEDGMDYWGKGTLVT
VSSEPKTPKPQAIAGGGGSGGGGSGGGGSLQGQVQLVETGGGLVKPGGSLRLSCVVSGFT
FDDYRMAWVRQAPGKELEWVSSIDSWSINTYYEDSVKGRFTISTDNAKNTLYLQMSSLKP
EDTAVYYCAAEDRLGVPTINAHPSKYDYNYWGQGTQVTVSSEPKTPKPQ
SEQ ID NO: 142
CAGGTGCAGCTGGTGGAGACGGGGGGAGGATTGGTGCAGGCTGGGGGCTCTCTGAGACTC
TCCTGTGCAGCCTCTGGACGCAGTTATGCCATGGGCTGGTTCCGCCAGGGTCCAGGGAAG
GAGCGTGAGTTTGTAGCCACTATCAGTTGGAGTAGTACTAACACATGGTATGCAGATTCC
GTGAAGGGCCGATTCACCATCTCTAGAGACAACGCCAAGAACACGGTGTATCTGCAAATG
AACAGCCTGAAACCTGAGGACACGGCTGTTTATTACTGTGCAGCGAGCCATCGTTTTAGC
GACTATCCCATGAGGTCAGAGGACGGCATGGACTACTGGGGCAAAGGGACCCTGGTCACC
GTCTCCTCAGAACCCAAGACACCAAAACCACAAGCGATCGCTGGTGGAGGCGGTTCAGGC
GGAGGTGGCTCTGGCGGTGGCGGTTCCCTGCAGGGTCAGGTGCAGCTGGTGGAGACTGGT
GGAGGCTTGGTGAAGCCTGGGGGTTCTCTGAGACTCTCCTGTGTAGTCTCCGGATTCACT
TTTGATGATTATCGCATGGCTTGGGTCCGCCAGGCTCCAGGGAAGGAGCTGGAGTGGGTG
TCCAGTATAGATAGTTGGAGTATCAACACATACTATGAAGACTCCGTGAAGGGCCGGTTC
ACCATCTCCACAGACAACGCCAAGAATACACTGTATCTGCAAATGAGCAGCCTGAAACCT
GAGGACACGGCCGTGTATTACTGTGCAGCAGAGGACCGCTTAGGTGTACCGACTATTAAC
GCCCACCCTTCAAAATATGATTATAACTACTGGGGGCAGGGGACCCAGGTCACCGTCTCC
TCAGAACCCAAGACACCAAAACCACAA
JLE-G6/JLE-E9,
SEQ ID NO: 143
QLQLVETGGGLVKPGGSLRLSCVVSGFTEDDYRMAWVRQAPGKELEWVSSIDSWSINTYY
EDSVKGRFTISTDNAKNTLYLQMSSLKPEDTAVYYCAAEDRLGVPTINAHPSKYDYNYWG
QGTQVTVSSEPKTPKPQAIAGGGGSGGGGSGGGGSLQGQVQLVETGGGLVQAGGSLRLSC
AASGRTESSYSMGWFRQAPGKEREYVAAVNSNGDSTFYADSIKGRFTVSRDAAKNTVYLQ
MNSLKPEDTALYYCAAVYGRYTYQSPKSYEYWGQGTQVTVSSEPKTPKPQ
SEQ ID NO: 144
CAGTTGCAGCTCGTGGAGACTGGTGGAGGCTTGGTGAAGCCTGGGGGTTCTCTGAGACTC
TCCTGTGTAGTCTCCGGATTCACTTTTGATGATTATCGCATGGCTTGGGTCCGCCAGGCT
CCAGGGAAGGAGCTGGAGTGGGTGTCCAGTATAGATAGTTGGAGTATCAACACATACTAT
GAAGACTCCGTGAAGGGCCGGTTCACCATCTCCACAGACAACGCCAAGAATACACTGTAT
CTGCAAATGAGCAGCCTGAAACCTGAGGACACGGCCGTGTATTACTGTGCAGCAGAGGAC
CGCTTAGGTGTACCGACTATTAACGCCCACCCTTCAAAATATGATTATAACTACTGGGGG
CAGGGGACCCAGGTCACCGTCTCCTCAGAACCCAAGACACCAAAACCACAAGCGATCGCT
GGTGGAGGCGGTTCAGGCGGAGGTGGCTCTGGCGGTGGCGGTTCCCTGCAGGGTCAGGTG
CAGCTGGTGGAGACGGGAGGAGGATTGGTGCAGGCTGGGGGCTCTCTGAGACTCTCGTGT
GCAGCCTCTGGACGCACCTTCAGTAGCTATTCCATGGGCTGGTTCCGCCAGGCTCCAGGG
AAGGAGCGTGAGTATGTAGCAGCAGTTAACTCCAATGGCGACAGTACATTCTATGCCGAC
TCCATTAAGGGCCGATTCACCGTCTCCAGAGACGCCGCCAAGAACACAGTCTATCTGCAA
ATGAACAGCCTGAAACCTGAGGACACGGCCCTTTATTACTGTGCAGCTGTCTACGGTAGA
TACACTTACCAGTCCCCAAAATCGTATGAGTACTGGGGCCAGGGGACCCAGGTCACCGTC
TCCTCAGAACCCAAGACACCAAAACCACAA

1.-25. (canceled)

26. A recombinant binding protein that specifically binds to a Bacillus anthracis (B. anthracis) toxin wherein the binding protein comprises an amino acid sequence selected from SEQ ID NO: 73, SEQ ID NO: 75, SEQ ID NO: 77, SEQ ID NO: 79, SEQ ID NO: 83, SEQ ID NO: 85, SEQ ID NO: 87, SEQ ID NO: 89, SEQ ID NO: 91, SEQ ID NO: 95, SEQ ID NO: 97, SEQ ID NO: 99, SEQ ID NO: 101, or a combination thereof.

27. The binding protein of claim 26, which specifically binds to at least one of an Anthrax surface protective antigen (PA), an Anthrax lethal factor (LF) or an Anthrax edema factor (EF) toxin of B. anthracis.

28. The binding protein of claim 27, wherein the binding protein inhibits or prevents transport or translocation of one or both of the LF and EF toxins into host cell cytosol.

29. The binding protein of claim 26, wherein the binding protein is heteromultimeric and comprises a plurality of non-identical binding regions, wherein each binding region specifically binds to a non-overlapping portion of the B. anthracis toxin.

30. The binding protein of claim 26, wherein the binding protein is dimeric and comprises two non-identical binding regions, wherein each binding region specifically binds to a non-overlapping portion of the B. anthracis toxin.

31. The binding protein of claim 26, wherein the binding protein further comprises at least one of a tag epitope that is specifically bound by an anti-tag antibody and a linker that separates the binding regions of the binding protein, wherein the linker comprises a peptide or a protein.

32. The binding protein of claim 31, wherein the linker is a peptide comprising GGGGS as set forth in SEQ ID NO: 147 or (GGGGS)₃ as set forth in SEQ ID NO: 145.

33. A polynucleotide encoding the binding protein of claim 26.

34. A composition comprising the binding protein of claim 26.

35. The composition of claim 34, further comprising a pharmaceutically acceptable carrier, excipient, or diluent.

36. A composition comprising the polynucleotide of claim 33.

37. The composition of claim 36, further comprising a pharmaceutically acceptable carrier, excipient, or diluent.

38. A method of detecting a presence of B. anthracis toxin in a sample, the method comprising:
contacting a test sample with the binding protein of claim 26 under conditions to form a complex; and
detecting binding between the binding protein and B. anthracis toxin in the sample by measuring the amount of complex formed, thereby detecting the presence of B. anthracis toxin in the sample.

39. The method of claim 38, wherein the test sample is selected from a medical sample, a food sample, a beverage sample, a water sample, or an environmental sample.

40. The method of claim 38, wherein the medical sample is selected from blood, plasma, tissue, stool, urine, perspiration, serum, semen, breast milk, cerebrospinal fluid, skin, or hair.

41. A method of therapeutically or prophylactically treating disease caused by B. anthracis toxin infection in a subject, the method comprising administering to a subject in need thereof, an effective amount of the pharmaceutical composition of claim 35 to therapeutically or prophylactically treat the B. anthracis toxin infection.

42. The method of claim 41, wherein the subject has been exposed to, or is at risk of exposure to, B. anthracis toxin.

43. The recombinant binding protein of claim 27, wherein the binding protein comprising an amino acid sequence selected from SEQ ID NO: 73, SEQ ID NO: 75, or a combination thereof, specifically binds to the EF toxin of B. anthracis.

44. The recombinant binding protein of claim 27, wherein the binding protein comprising an amino acid sequence selected from SEQ ID NO: 77, SEQ ID NO: 79, SEQ ID NO: 83, SEQ ID NO: 85, SEQ ID NO: 87, SEQ ID NO: 89, or a combination thereof, specifically binds to the LF toxin of B. anthracis.

45. The recombinant binding protein of claim 27, wherein the binding protein comprising an amino acid sequence selected from SEQ ID NO: 91, SEQ ID NO: 95, SEQ ID NO: 97, SEQ ID NO: 99, SEQ ID NO: 101, or a combination thereof, binds to both the EF toxin and the LF toxin of B. anthracis.
